SEQUENCE LISTING (24 sequences)

SEQ ID NO: 1 - DNA, 25 bp, Artificial Sequence
  Synthetic oligonucleotide
caccgaggagactcgtccatacacg

SEQ ID NO: 2 - DNA, 25 bp, Artificial Sequence
  Synthetic oligonucleotide
ctcctctgagcaggtatgtgccaaa

SEQ ID NO: 3 - DNA, 25 bp, Artificial Sequence
  Synthetic oligonucleotide
caccgtgatgaggaacgggcgaaga

SEQ ID NO: 4 - DNA, 25 bp, Artificial Sequence
  Synthetic oligonucleotide
cactactccttgcccgcttctcaaa

SEQ ID NO: 5 - DNA, 20 bp, Artificial Sequence
  Synthetic oligonucleotide
gtgagcctcaaccgcatcct

SEQ ID NO: 6 - DNA, 20 bp, Artificial Sequence
  Synthetic oligonucleotide
aggatgcggttgaggctcac

SEQ ID NO: 7 - DNA, 20 bp, Artificial Sequence
  Synthetic oligonucleotide
cggaacagcttctggatgaa

SEQ ID NO: 8 - DNA, 20 bp, Artificial Sequence
  Synthetic oligonucleotide
ttcatccagaagctgttccg

SEQ ID NO: 9 - DNA, 20 bp, Artificial Sequence
  Synthetic oligonucleotide
agagtgtgtacttacagcgc

SEQ ID NO: 10 - DNA, 20 bp, Artificial Sequence
  Synthetic oligonucleotide
gcgctgtaagtacacactct

SEQ ID NO: 11 - DNA, 20 bp, Artificial Sequence
  Synthetic oligonucleotide
gaggatcatgctcctagaac

SEQ ID NO: 12 - DNA, 20 bp, Artificial Sequence
  Synthetic oligonucleotide
gttctaggagcatgatcctc

SEQ ID NO: 13 - DNA, 19 bp, Artificial Sequence
  Synthetic primer
atggaggggaatacagccc

SEQ ID NO: 14 - DNA, 20 bp, Artificial Sequence
  Synthetic primer
ttctttgcagctccttcgtt

SEQ ID NO: 15 - DNA, 20 bp, Artificial Sequence
  Synthetic primer
cctgtagcccacgtcgtagc

SEQ ID NO: 16 - DNA, 22 bp, Artificial Sequence
  Synthetic primer
agcaatgactccaaagtagacc

SEQ ID NO: 17 - DNA, 20 bp, Artificial Sequence
  Synthetic primer
cacagcagcacatcaacaag

SEQ ID NO: 18 - DNA, 20 bp, Artificial Sequence
  Synthetic primer
gtgctcatgtcctcatcctg

SEQ ID NO: 19 - DNA, 23 bp, Artificial Sequence
  Synthetic primer
gaggataccactcccaacagacc

SEQ ID NO: 20 - DNA, 24 bp, Artificial Sequence
  Synthetic primer
aagtgcatcatcgttgttcataca

SEQ ID NO: 21 - DNA, 22 bp, Artificial Sequence
  Synthetic primer
ccaatcttgcagtcgtgtttgt

SEQ ID NO: 22 - DNA, 30 bp, Artificial Sequence
  Synthetic primer
catctccaaatagttgatgtattcttgaac

SEQ ID NO: 23 - DNA, 20 bp, Artificial Sequence
  Synthetic primer
cagactgccagcctaagaca

SEQ ID NO: 24 - DNA, 20 bp, Artificial Sequence
  Synthetic primer
aggaattgcttgttgatccc

(19) United States
(12) Patent Application Publication
(10) Pub. No.: US 2022/0104468 A1
(43) Pub. Date: Apr. 7, 2022

(54) MODULATION OF LC3-ASSOCIATED ENDOCYTOSIS PATHWAY AND GENETICALLY MODIFIED NON-HUMAN ANIMALS AS A MODEL OF NEUROINFLAMMATION AND NEURODEGENERATION

(71) Applicant: St. Jude Children's Research Hospital, Memphis, TN (US)

(72) Inventors: Bradlee L. Heckmann, Memphis, TN (US); Douglas R. Green, Memphis, TN (US)

(73) Assignee: St. Jude Children's Research Hospital, Memphis, TN (US)

(21) Appl. No.: 17/425,106

(22) PCT Filed: Jan. 22, 2020
(86) PCT No.: PCT/IB2020/050504

§ 371 Date: Jul. 22, 2021

Related U.S. Application Data
(60) Provisional application No. 62/795,217, filed on Jan. 22, 2019.
(60) Provisional application No. 62/797,564, filed on Jan. 28, 2019.

(51) Int. Cl. A01K 67/027; A61K 38/17; A61P 25/28; G01N 33/50

(52) U.S. Cl. CPC A01K 67/0276; A61K 38/1709; A61P 25/28; G01N 33/5023; G01N 2800/2821; A01K 2217/075; A01K 2217/206; A01K 2227/105; A01K 2267/03; G01N 33/5088

(57) ABSTRACT

Compositions and methods are provided for modifying and treating neuroinflammatory and neurodegenerative diseases. The methods and compositions can be used to ameliorate the effects of a deficiency in the LC3-associated endocytosis (LANDO) pathway for clearing β-amyloid. Thus, methods are further provided for modulating β-amyloid clearance using an effective amount of a pharmaceutical composition that targets the LANDO pathway. Accordingly, pharmaceutical compositions that target the LANDO pathway are provided herein. The methods and compositions described herein can be used to treat neuroinflammatory and neurodegenerative diseases, such as Alzheimer's disease.

### FEDERALLY SPONSORED RESEARCH OR DEVELOPMENT

This invention was made with government support under grants AI040646 and AI138492, awarded by the National Institutes of Health. The government has certain rights in the invention.

### FIELD OF THE INVENTION

The invention relates to the field of cell biology and immunology. In particular, the invention relates to methods and compositions for modulating the LC3-associated endocytosis (LANDO) pathway in order to reduce neuroinflammation and neurodegeneration in subjects. The methods and compositions can be used to treat neuroinflammation and neurodegeneration in LANDO-deficient subjects.

### REFERENCE TO A SEQUENCE LISTING SUBMITTED ELECTRONICALLY AS A TEXT FILE

The instant application contains a Sequence Listing which has been submitted in ASCII format via EFS-Web and is hereby incorporated by reference in its entirety. Said ASCII copy, created on Jan. 28, 2019, is named S884351230USP200464SEQLIST.txt, and is 8 KB in size.

### BACKGROUND OF THE INVENTION

Microglial cells are the primary immune cell of the central nervous system (CNS) and account for approximately 10-15% of all cells found in the brain. As resident macrophage-like cells they provide the first form of active immune defense for the CNS (Lenz and Nelson (2018) Front Immunol 9:698). Like other resident and peripheral macrophages, microglia have the ability to recognize pathogens and other inflammatory stimulants by virtue of a host of receptors including toll-like receptors (TLRs) (Gurley et al. (2008) PPAR Res 453120), Fc receptors (Fuller et al. (2014) Front Neurosci 8:235), Ig-superfamily receptors including TREM2 (Ulrich et al. (2014) Mol Neurodegener 9:20; Wang et al. (2016) J Exp Med 213:667-675; Zhao et al. (2018) Neuron 97:1023-1031), scavenger receptors (SR) (Wilkinson and El Khoury, 2012) and complement receptors (Doens and Fernandez (2014) J Neuroinflammation 11:48). It is currently believed that cooperation between several of these receptor families is responsible for the recognition of and response to amyloid, specifically β-amyloid (A3) by microglial cells (Doens and Fernandez (2014); Liu et al. (2012) J Immunol 188:1098-1107). Upon recognition and binding of ligands such as A3, microglial cells internalize the target by receptor-mediated endocytosis, leading to activation of signaling pathways and specific cytokine production in a ligand-dependent manner (Dheen et al. (2007) Curr Med Chem 14:1189-1197). Like other macrophages, microglia possess the ability to act in both pro- and anti-inflammatory capacities depending upon their polarization state. Microglia can undergo both classical (M1) and alternative (M2) activation dependent on what cell surface immune receptors are engaged in response to peripheral signal recognition, resulting in the activation of multiple downstream intracellular signaling pathways (Wang et al. (2014) Front Immunol 5:614). As a consequence, production of pro- or anti-inflammatory cytokines occurs. Elegant studies have demonstrated that microglia are the principal mediators of inflammation occurring in response to amyloid accumulation (Machado et al. (2016) Int J Mol Sci 17; Perry and Holmes (2014) Nat Rev Neurol 10:217-224; Wang et al. (2015) Ann Transl Med 3:136). Microglia and their contribution to neuroinflammation are highly correlated to the progression of neurodegeneration and synaptic dysfunction, particularly with respect to Alzheimer's disease (AD). When combined with the primary insult of amyloid accumulation and neurofibrillary tangle formation, pro-inflammatory cytokines and chemokines secreted into the immediate neurological environment accelerate neuronal injury and eventually neuron death (Aktas et al. (2007) Arch Neurol 64:185-189; Heckmann et al. (2018) Cell Death Differ 26(1):41-52; Morales et al. (2014) Front Cell Neurosci 8:112). There remains a need for understanding the molecular mechanisms by which microglia control β-amyloid clearance and inflammatory signaling in order to identify novel therapeutics for targeting neuroinflammation and neurodegeneration, particularly in the context of Alzheimer's disease.

### SUMMARY OF THE INVENTION

Compositions and methods are provided for modifying and treating neuroinflammatory and neurodegenerative disease. The methods and compositions can be used to ameliorate the effects of a deficiency in the LANDO pathway for clearing β-amyloid (Aβ). Thus, methods are further provided for modulating Aβ clearance using an effective amount of a pharmaceutical composition that targets the LANDO pathway. Accordingly, pharmaceutical compositions that target the LANDO pathway and methods for identifying such compounds are provided herein. The methods and compositions described herein can be used to treat neuroinflammatory or neurodegenerative disease, such as Alzheimer's disease. A genetically modified non-human animal model of neuroinflammation and neurodegeneration comprising microglial LANDO knockdown or knockout is also provided, along with methods of making the same. The non-human animal model finds use in studying neuroinflammation, neurodegeneration, Alzheimer's disease, β-amyloid deposition and clearance, or the LANDO pathway and in screening compounds for the modulation of the same.

### BRIEF DESCRIPTION OF THE FIGURES

FIG. 1 depicts LysM-cre mediated abrogation of FIP200 and ATG5 expression. FIGS. 1A and 1B show LysM-cre mediated reduction in FIP200 and ATG5 in primary microglia isolated from the indicated genotypes as measured by immunoblot (FIG. 1A) and qPCR (FIG. 1B). For FIG. 1B, n=4 mice for both cre⁻, n=5 mice for FIP200^{fl/fl}cre⁺ and ATG5^{fl/fl}cre⁺. Quantitative PCR was performed in triplicate. FIG. 1C shows the analysis of autophagic capacity in primary microglia isolated from FIP200^{fl/fl} and ATG5^{fl/fl} cre⁺ or − mice as indicated. Cells were treated with rapamycin for 12 hours. Autophagic activation was determined by LC3-lipidation by immunoblot. FIG. 1D provides representative images showing β-amyloid accumulation in the cortex of myeloid ATG5-deficient 5×FAD mice. FIG. 1E depicts the quantification of cortical β-amyloid deposition in FIP200 and ATG5-deficient 5×FAD mice. Each point represents an individual mouse. Data are represented as mean±SEM. Significance was calculated using Student's t-test. *p<0.05, ***p<0.001.

FIG. 2 shows that ATG5 and Rubicon-deficiency exacerbates β-amyloid deposition. FIGS. 2A and 2B depict representative images for β-amyloid (red) in the hippocampus of 4 month-old 5×FAD mice with indicated genetic alterations. FIGS. 2C and 2D provide the quantification of β-amyloid plaque number (FIG. 2C) and plaque area (FIG. 2D) in the hippocampus of 4 month-old 5×FAD mice. Each point represents average quantification from one mouse. FIG. 2E provides representative images for β-amyloid (red) deposition in the 5^{th} cortical layer in 4 month-old 5×FAD mice. FIG. 2F shows the quantification of β-amyloid plaque number in the cortex of 4 month-old 5×FAD mice. Each point represents average quantification from one mouse. Data are represented as mean±SEM. Significance was calculated using Student's t-test. **p<0.01, ****p<0.0001.

FIG. 3 demonstrates the characterization of BV2 microglia lacking FIP200, ATG5, and Rubicon. FIG. 3A depicts an immunoblot analysis showing successful depletion of FIP200, ATG5, or Rubicon as indicated in BV2 microglia by CRISPR/Cas9. FIG. 3B depicts a three-dimensional reconstruction demonstrating Aβ1-42 induced recruitment of LC3 to oligomeric β-amyloid. FIG. 3C shows the quantification of receptor internalization for receptor recycling assays (FIG. 4) in BV2 microglia. Each point represents a unique experiment performed in duplicate. FIG. 3D provides representative images showing uptake of zymosan, dextran, or β-amyloid in parental BV2 microglia in the presence or absence of the phagocytosis inhibitor latrunculin A (50 μM). FIG. 3E provides the quantification of membrane-associated LC3 by flow cytometry in vehicle or latrunculin A (LA) treated parental BV2 microglia in response to either zymosan or β-amyloid. n=3 for each condition performed in duplicate. FIG. 3F provides representative images showing zymosan or β-amyloid co-localization with LAMP1 labeled lysosomes in BV2 microglia of the indicated genotypes. Data are represented as mean±SEM. Significance was calculated using Student's t-test. **p<0.01.

FIG. 4 shows that ATG5 and Rubicon-deficiency impairs LANDO and recycling of β-amyloid receptors. FIG. 4A provides representative images showing that GFP-LC3-recruitment to β-amyloid (red) containing endosomes in BV2 microglia is dependent on ATG5 and Rubicon, but not FIP200. White arrows indicate LC3+ endosomes. FIG. 4B provides the quantification of membrane-associated GFP-LC3 in BV2 microglia following stimulation with 1 μM oligomeric TAMRA-Aβ1-42. GFP-LC3 was assayed using flow cytometry. Each point represents one independent experiment performed in triplicate. FIG. 4C shows the quantification of zymosan (4:1, particle:cell), dextran (500 ng/ml), or β-amyloid (1 μM) uptake in BV2 microglia treated with either a vehicle or 50 μM latrunculin A (LA). All substrates were fluorescently labeled as follows, zymosan (AF594), dextran (Texas Red), and β-amyloid (TAMRA). MFI was measured by flow cytometry. n=3 per condition performed in duplicate. FIG. 4D provides the results of a pulse-chase based, β-amyloid clearance assay performed in BV2 microglia treated with oligomeric TAMRA-Aβ1-42. Clearance of β-amyloid was monitored by flow cytometry. n=4 per genotype performed in duplicate. FIG. 4E shows the quantification of the co-localization between zymosan or β-amyloid and LAMP1 labeled lysosomes in BV2 microglia (see FIG. 3F). Co-localization was quantified using the Manders coefficient. n=3 per genotype performed in duplicate. FIG. 4F shows primary and secondary uptake of β-amyloid measured in BV2 microglia. Oligomeric Alexafluor 488-Aβ1-42 was used for primary uptake and TAMRA-Aβ1-42 was used for secondary uptake. Internalization of β-amyloid was monitored by flow cytometry and MFI was quantified for each step. Each point represents one independent experiment performed in duplicate. FIG. 4G provides representative images of receptor recycling for TLR4, TREM2, and CD36 in BV2 microglia. FIG. 4H shows the quantification of recycled receptors in BV2 microglia. Each point is one independent experiment performed in duplicate. FIG. 4I provides representative images of TREM2 recycling in primary microglia from Rubicon^{+/-} or Rubicon^{-/-} mice. FIG. 4J shows the quantification of TREM2 recycling in primary microglia from indicated genotypes. Each point is one independent experiment performed in duplicate. Data are represented as mean±SEM. Significance was calculated using Student's t-test. *p<0.05, **p<0.01, ***p<0.001.

FIG. 5 shows that recycling of CD36, TREM2, and TLR4 in RAW264.7 and BMDMs is LANDO-dependent. FIG. 5A depicts an immunoblot analysis showing either CRISPR/Cas9-mediated depletion or retroviral mediated overexpression of the indicated genes in RAW264.7 cells. FIGS. 5B and 5C provide representative imaging of receptor recycling in (FIG. 5B) RAW264.7 cells and (FIG. 5C) primary BMDMs.

FIG. 6 shows that abrogation of LANDO promotes β-amyloid induced inflammation. FIG. 6A depicts the quantification of receptor recycling in RAW264.7 cells deficient in the indicated genes as shown or overexpressing RavZ or dominant-negative ATG4 as shown. Each data point represents a unique experiment performed in duplicate. FIG. 6B shows the quantification of receptor recycling in BMDMs isolated from the indicated genotypes and for the indicated receptors. Each data point represents a unique experiment performed in duplicate. FIGS. 6C and 6D depict pro-inflammatory cytokine expression in (FIG. 6C) BMDMs or (FIG. 6D) BV2 microglia in response to oligomeric Aβ1-42 measured by qPCR. For FIG. 6C, n=3 per genotypes performed in duplicate. For FIG. 6D, n=4 per genotype performed in triplicate. FIG. 6E depicts qPCR analysis of pro-inflammatory gene expression in primary microglia following oligomeric Aβ1-42 exposure. n=3 per genotype performed in triplicate. FIG. 6F depicts cytokine production by primary microglia in response to oligomeric Aβ1-42 measured by ELISA. n=3 per genotype performed in duplicate. Data are represented as mean±SEM. Significance was calculated using Student's t-test. *p<0.05, **p<0.01, ***p<0.001.

FIG. 7 shows that LANDO decreases β-amyloid induced reactive microgliosis. FIG. 7A depicts representative images showing microglial activation (green-Iba1 positive) in the hippocampus and the 5^{th} cortical layer (cortex) of the indicated 5×FAD genotypes. FIGS. 7B and 7C show the quantification of activated microglia in the hippocampus (FIG. 7B) and cortex (FIG. 7C) respectively. Each point represents an individual mouse. FIG. 7D shows representative images indicating microglial (green) morphology. FIG. 7E depicts the quantification of ramified vs. ameboid microglia in the indicated 5×FAD genotypes. Each point represents an individual mouse. FIG. 7F provides representative images and quantification of microglia/plaque-association in Rubicon^{+/-} or Rubicon^{-/-} mice. Each point represents an individual mouse. FIG. 7G provides results from a qPCR analysis of inflammatory gene expression in hippocampal slices from 5×FAD Rubicon^{+/-} or Rubicon^{-/-} mice. n=7 mice per genotype, qPCR performed in triplicate. Data are represented as mean±SEM. Significance was calculated using Student's t-test. *p<0.05, **p<0.01, ***p<0.001.

FIG. 8 shows the analysis of infiltrating monocytes versus resident microglia in 5×FAD Rubicon-deficient mice. FIG. 8A provides representative flow cytometric analysis of resident microglia versus peripheral monocytes. Expression of the microglia-specific receptor TMEM119 was analyzed on the total CD11b monocytic pool (containing all monocytes present in the brain) to delineate peripheral cells (TMEM119−) versus resident microglia (TMEM119+). FIG. 8B shows the quantification of the percentage of infiltrating monocytes. n=4 mice per genotype. FIG. 8C provides representative histogram and quantification of microglia activation by Iba1 expression on TMEM119+ cells from 5×FAD Rubicon^{+/-} and Rubicon^{-/-} mice. n=4 mice per genotype. Data are represented as mean±SEM. Significance was calculated using Student's t-test. ***p<0.001.

FIG. 9 shows that LANDO mitigates tau hyperphosphorylation. FIGS. 9A and 9B provide representative images showing hyperphosphorylation of tau at S202/T205 in the hippocampus (FIG. 9A) and cortex (FIG. 9B) of LANDO-deficient 5×FAD mice. FIGS. 9C and 9D provide the quantification of phospho-tau in the hippocampus (FIG. 9C) and cortex (FIG. 9D) of the indicated 5×FAD genotypes. Each point represents an individual mouse. Data are represented as mean±SEM. Significance was calculated using Student's t-test. *p<0.05, **p<0.01, ***p<0.001.

FIG. 10 shows that LANDO-deficiency promotes β-amyloid induced neuronal death. FIG. 10A provides representative images showing neurons (NeuN-green) in the hippocampus of the indicated 5×FAD genotypes. FIG. 10B provides a quantification of neuronal content within the hippocampus. Each point represents an individual mouse. FIG. 10C depicts representative images identifying neuronal apoptosis within the CA3-region of the hippocampus of 5×FAD Rubicon-deficient mice. FIG. 10D shows the quantification of apoptotic neurons within the hippocampus of 5×FAD Rubicon-deficient mice. Each point represents an individual mouse. FIGS. 10E and 10F provide an analysis of hippocampal synaptic transmission (FIG. 10E) and long-term potentiation (FIG. 10F) in 5×FAD Rubicon-deficient mice. n=9 mice per genotype with a minimum of 5 slices per mouse. Data are represented as mean±SEM. Significance was calculated using Student's t-test. *p<0.05, **p<0.01, ***p<0.001.

FIG. 11 shows that loss of CA3 neurons in LANDO-deficient mice leads to behavior and memory impairment. FIGS. 11A and 11B provide the results of a sucrose preference test (FIG. 11A) and fluid intake measurement (FIG. 11B) for the indicated 5×FAD genotypes. Each data point represents an individual mouse. FIGS. 11C and 11D show the results for a Y-maze test for short-term memory measuring spontaneous arm alternation (FIG. 11C) and total arm entries (FIG. 11D) in the indicated 5×FAD genotypes. Each data point represents an individual mouse. FIGS. 11E-11G provide an analysis of novel object recognition measuring total exploration time (FIG. 11E), preference for the novel object (FIG. 11F), and the ability to discriminate (FIG. 11G) in 5×FAD Rubicon^{+/-} or Rubicon^{-/-} mice. Each data point represents an individual mouse. Data are represented as mean±SEM. Significance was calculated using Student's t-test. *p<0.05, ***p<0.001, ****p<0.0001.

FIG. 12 shows Aβ pathology in Atg16L^{ΔWD} mice (mice lacking the WD-domain of Atg16L) aged to two years. FIG. 12A provides representative micrographs showing immunofluorescence imaging of Aβ in hippocampus of Atg16L^{ΔWD} mice (FIG. 12A, right panel) and control (Atg16L^{+/+}) mice (FIG. 12A, left panel). FIG. 12B is a graph depicting quantification of Aβ mean fluorescence intensity (Aβ MFI) in hippocampus of Atg16L^{ΔWD} mice and control (Atg16L^{+/+}) mice; each point on the graph representing an individual mouse. FIG. 12C provides representative micrographs showing immunofluorescence imaging of Aβ in cerebral cortex of Atg16L^{ΔWD} mice (FIG. 12C, right panel) and control (Atg16L^{+/+}) mice (FIG. 12C, left panel). FIG. 12D is a graph depicting quantification of number of plaques (individually measurable accumulations of Aβ) in cerebral cortex; each point on the graph representing an individual mouse. FIG. 12E is a graph depicting quantification of Aβ mean fluorescence intensity (Aβ MFI) in cerebral cortex of Atg16L^{ΔWD} mice and control (Atg16L^{+/+}) mice; each point on the graph representing an individual mouse. FIG. 12F provides representative micrographs showing high resolution immunofluorescence imaging of extracellular Aβ deposits (FIG. 12F, upper panel) and intraneuronal Aβ deposits (FIG. 12F, lower panel) in hippocampus of Atg16L^{ΔWD} mice. ****p<0.0001

FIG. 13 shows Tau pathology in Atg16L^{ΔWD} mice (mice lacking the WD-domain of Atg16L) aged to two years. FIG. 13A provides representative micrographs showing immunofluorescence imaging of S199/202 Tau phosphorylation in hippocampus of Atg16L^{ΔWD} mice (FIG. 13A, right panel) and control (Atg16L^{+/+}) mice (FIG. 13A, left panel). FIG. 13B is a graph depicting quantification of Tau phosphorylation at S199/202 (expressed as pTau MFI) in hippocampus of Atg16L^{ΔWD} mice and control (Atg16L^{+/+}) mice; each point on the graph representing an individual mouse. FIG. 13C provides representative micrographs showing CA3-field specific imaging of S199/202 Tau phosphorylation in Atg16L^{ΔWD} mice (FIG. 13C, right panel) and control (Atg16L^{+/+}) mice (FIG. 13C, left panel). FIG. 13D provides representative immunoblots depicting expression of S199/202 phosphorylated Tau (pTau) and total Tau in whole brain lysate of Atg16L^{ΔWD} mice and control (Atg16L^{+/+}) mice. Actin was used as a loading control. FIG. 13E provides representative micrographs showing immunofluorescence imaging of S199/202 Tau phosphorylation in cerebral cortex of Atg16L^{ΔWD} mice (FIG. 13E, right panel) and control (Atg16L^{+/+}) mice (FIG. 13E, left panel). FIG. 13F is a graph depicting quantification of Tau phosphorylation at S199/202 (expressed as pTau MFI) in cerebral cortex of Atg16L^{ΔWD} mice and control (Atg16L^{+/+}) mice; each point on the graph representing an individual mouse. ***p<0.001

FIG. 14 shows impairment in LANDO-dependent recycling of the putative Aβ receptors TREM2, CD36, and TLR4 and the effect of this impairment on secondary uptake of Aβ in Atg16L^{ΔWD} mice (mice lacking the WD-domain of Atg16L) aged to two years. FIG. 14A provides representative micrographs showing immunofluorescence imaging of receptor recycling for TREM2, CD36, and TLR4 in primary microglia of Atg16L^{ΔWD} mice (FIG. 14A, lower panel) and control (Atg16L^{+/+}) mice (FIG. 14A, upper panel). FIG. 14B is a graphical representation of quantification of receptor recycling for TREM2, CD36, and TLR4 in primary microglia of Atg16L^{ΔWD} mice and control (Atg16L^{+/+}) mice, depicted as fluorescent area/total cell number. n=4 performed in triplicate. FIG. 14C is a graphical representation of quantification of secondary Aβ uptake measured in primary microglial cells of Atg16L^{ΔWD} mice and control (Atg16L^{+/+}) mice. n=3 performed in triplicate. ** p<0.01

FIG. 15 shows microgliosis and neuroinflammation in Atg16L^{ΔWD} mice (mice lacking the WD-domain of Atg16L) aged to two years. FIG. 15A provides representative micrographs showing immunofluorescence imaging of microglial activation in hippocampus of Atg16L^{ΔWD} mice (FIG. 15A, right panel) and control (Atg16L^{+/+}) mice (FIG. 15A, left panel), as measured by Iba1. FIG. 15B is a graph depicting quantification of Iba1 mean fluorescent intensity (Iba1 MFI) in the hippocampus of Atg16L^{ΔWD} mice and control (Atg16L^{+/+}) mice; each point on the graph representing an individual mouse. FIG. 15C provides representative micrographs showing immunofluorescence imaging of microglial activation in cerebral cortex of Atg16L^{ΔWD} mice (FIG. 15C, right panel) and control (Atg16L^{+/+}) mice (FIG. 15C, left panel), as measured by Iba1. FIG. 15D is a graph depicting quantification of Iba1 mean fluorescent intensity (Iba1 MFI) in the cerebral cortex of Atg16L^{ΔWD} mice and control (Atg16L^{+/+}) mice; each point on the graph representing an individual mouse. FIG. 15E provides representative micrographs showing morphological analysis of microglia marked by Iba1 in Atg16L^{ΔWD} mice (FIG. 15E, right panel) and control (Atg16L^{+/+}) mice (FIG. 15E, left panel). FIG. 15F is a graph depicting relative expression of IL1β, TNFα, and IL6 in hippocampus of Atg16L^{ΔWD} mice and control (Atg16L^{+/+}) mice, as determined by qPCR analysis. n=5 performed in triplicate. ***p<0.001, ** p<0.01, * p<0.05

FIG. 16 shows neurodegeneration in Atg16L^{ΔWD} mice (mice lacking the WD-domain of Atg16L) aged to two years. FIG. 16A provides representative micrographs showing immunofluorescence imaging of neuronal cleaved caspase 3 staining in Atg16L^{ΔWD} mice (FIG. 16A, right panel) and control (Atg16L^{+/+}) mice (FIG. 16A, left panel). FIG. 16B provides representative micrographs showing immunofluorescence imaging of CA3-field cleaved caspase 3 in neurons in Atg16L^{ΔWD} mice (FIG. 16B, right panel) and control (Atg16L^{+/+}) mice (FIG. 16B, left panel). FIG. 16C is a graph showing quantification of cleaved caspase 3 mean fluorescent intensity (cCASP3 MFI) in hippocampus of Atg16L^{ΔWD} mice and control (Atg16L^{+/+}) mice; each point on the graph representing an individual mouse. FIG. 16D provides representative micrographs showing imaging of neuronal TUNEL staining in the CA3-field of Atg16L^{ΔWD} mice (FIG. 16D, right panel) and control (Atg16L^{+/+}) mice (FIG. 16D, left panel). FIG. 16E provides representative micrographs showing imaging of neuronal nuclei staining in the hippocampus of Atg16L^{ΔWD} mice (FIG. 16E, right panel) and control (Atg16L^{+/+}) mice (FIG. 16E, left panel). FIG. 16F is a graph showing quantification of total neuron number (Neuron #) in hippocampus of Atg16L^{ΔWD} mice and control (Atg16L^{+/+}) mice; each point on the graph representing an individual mouse. *** p<0.001, ** p<0.01

FIG. 17 shows impaired synaptic plasticity and behavioral deficiency in Atg16L^{ΔWD} mice (mice lacking the WD-domain of Atg16L) aged to two years. FIG. 17A is a graph showing hippocampal electrophysiology measuring long-term potentiation in Atg16L^{ΔWD} mice and control (Atg16L^{+/+}) mice; n=9 mice per group with at least 4 slices per sample. FIG. 17B is a graph showing sucrose preference measured as a percentage compared to standard water in Atg16L^{ΔWD} mice and control (Atg16L^{+/+}) mice. FIG. 17C is a graph showing spontaneous alternation percentage as measured by Y-maze analysis in Atg16L^{ΔWD} mice and control (Atg16L^{+/+}) mice. FIG. 17D provides graphs showing novel object preference (FIG. 17D, left panel) and discrimination index (FIG. 17D, right panel), as measured by NOR analysis, in Atg16L^{ΔWD} mice and control (Atg16L^{+/+}) mice. FIG. 17E is a graph showing fluid intake as measured in grams/day during the sucrose preference test in Atg16L^{ΔWD} mice and control (Atg16L^{+/+}) mice; each point on the graph representing an individual mouse. FIG. 17F is a graph showing total number (#) of arm entries during Y-maze analysis in Atg16L^{ΔWD} mice and control (Atg16L^{+/+}) mice; each point on the graph representing an individual mouse. FIG. 17G is a graph showing total exploration time in seconds (s) during the NOR analysis in Atg16L^{ΔWD} mice and control (Atg16L^{+/+}) mice; each point on the graph representing an individual mouse. ****p<0.0001, ***p<0.001

FIG. 18 provides graphs comparing background strain of mice and markers of disease pathology. Single nucleotide polymorphism analysis was completed on mice used herein to determine background strain homogeneity. The background percentage of C57BL6 (B6) is represented as a color distribution. Background percentage was then correlated to disease markers including behavior, as measured by spontaneous alternation in the Y-maze, or Aβ deposition. Pure B6 wild-type is shown as a reference.

FIG. 19 shows therapeutic response in Atg16L WD-domain deficient mice (Atg16L^{ΔWD} mice) with established disease pathology following treatment with MCC950 or placebo for 8 weeks. FIG. 19A is a graph showing spontaneous alternation percentage as measured by Y-maze analysis in Atg16L^{ΔWD} mice and control (Atg16L^{+/+}) mice. FIG. 19B provides graphs showing novel object preference (FIG. 19B, left panel) and discrimination index (FIG. 19B, right panel), as measured by NOR analysis, in Atg16L^{ΔWD} mice and control (Atg16L^{+/+}) mice. FIG. 19C is a graph showing quantification of Iba1 total staining area as a surrogate for microglial activation in the hippocampus of Atg16L^{ΔWD} mice that were treated with MCC950 or placebo; each point on the graph representing an individual mouse. FIG. 19D provides representative micrographs showing immunofluorescence imaging of microglial activation by Iba1 staining in hippocampus of Atg16L^{ΔWD} mice that were treated with MCC950 (FIG. 19D, right panel) or placebo (FIG. 19D, left panel). FIG. 19E provides representative micrographs showing immunofluorescence imaging of Aβ staining in the hippocampus of Atg16L^{ΔWD} mice that were treated with MCC950 (FIG. 19E, right panel) or placebo (FIG. 19E, left panel). FIG. 19F provides representative micrographs showing immunofluorescence imaging of S199/202 Tau phosphorylation in CA3-field of the hippocampus of Atg16L^{ΔWD} mice that were treated with MCC950 (FIG. 19F, right panel) or placebo (FIG. 19F left panel). FIG. 19G provides representative micrographs showing neuronal TUNEL staining in the CA3-field of Atg16L^{ΔWD} mice that were treated with MCC950 (FIG. 19G, right panel) or placebo (FIG. 19G, left panel). FIG. 19H is a graph showing spontaneous alternation percentage as measured by Y-maze analysis in Atg16L^{+/+} mice and MCC950-treated or placebo-treated Atg16L^{ΔWD} mice. FIG. 19I provides graphs showing novel object preference (FIG. 19I, right panel) and discrimination index (FIG. 19I, left panel), as measured by NOR analysis in Atg16L^{+/+} mice and MCC950-treated or placebo-treated Atg16L^{ΔWD} mice. FIG. 19J is a graph showing total number (#) of arm entries during Y-maze analysis in Atg16L^{ΔWD} mice treated with MCC950 or placebo; each point on the graph representing an individual mouse. FIG. 19K is a graph showing total exploration time in seconds (s) during NOR analysis in Atg16L^{ΔWD} mice following treatment with MCC950 or placebo; each point on the graph representing an individual mouse. ****p<0.0001, *** p<0.001, ** p<0.01, * p<0.05

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### 1. Overview

Compositions and methods are provided herein for the treatment of conditions associated with a deficiency in the LC3-associated endocytosis (LANDO) pathway. LANDO is a newly discovered form of receptor-mediated endocytosis and receptor recycling characterized by the association of LC3 (light chain 3)/GABARAP (gamma-aminobutyric acid receptor-associated protein)-family proteins (herein, “LC3”) with endosomal membranes. LANDO in myeloid cells is a critical regulator of immune-mediated aggregate removal by receptor-mediated endocytosis and neuroinflammation.

Mice lacking LANDO but not canonical autophagy in the myeloid compartment (with a 5×FAD background in which the mice express transgenes of amyloid precursor protein and presenilin1 containing several mutations associated with human familial Alzheimer's disease) have a robust increase in pro-inflammatory cytokine production in the hippocampus and have increased levels of neurotoxic β-amyloid (Aβ) accumulation. This inflammation and Aβ deposition leads to reactive microgliosis and hyperphosphorylation of tau, a protein that is vital to neuronal structure and function. As a consequence, LANDO-deficient mice have increased neurodegeneration, resulting in impaired neuronal signaling and consequential behavioral and memory deficits. Thus, LANDO serves a protective role in myeloid cells of the central nervous system (CNS) in neurodegenerative pathologies resulting from β-amyloid deposition.

The LANDO pathway is distinct from the previously discovered LC3-associated phagocytosis (LAP) pathway, and also distinct from the canonical autophagy pathway. Macroautophagy (herein, autophagy or canonical autophagy) is a catabolic, cell survival mechanism activated during nutrient scarcity involving degradation and recycling of unnecessary or dysfunctional components. The proteins of autophagy machinery often interact with pathogens, such as Salmonella enterica, Listeria monocytogenes, Aspergillus fumigatus and Shigella flexneri, and function to quarantine and degrade invading organisms (xenophagy). LC3 (mammalian homologue of Atg8) is the most commonly monitored autophagy-related protein, and its lipidated form, LC3-II, is present on autophagosomes during canonical autophagy.

LC3-associated phagocytosis (LAP) is a process triggered following phagocytosis of particles that engage cell-surface receptors such as TLR1/2, TLR2/6, TLR4, TIM4 and FcR, resulting in recruitment of some, but not all, members of the autophagic machinery to stimulus-containing phagosomes, facilitating rapid phagosome maturation, degradation of engulfed pathogens, and modulation of immune responses. LAP and autophagy have been shown to be functionally and mechanistically distinct processes. Whereas the autophagosome is a double-membrane structure, the LAP-engaged phagosome (LAPosome) is composed of a single membrane. Autophagy requires the activity of the pre-initiation complex, but LAP does not. However, LAP requires some autophagic components, such as the Class III PI(3)K (phosphoinositide 3-kinase) complex and elements of the ubiquitylation-like, protein conjugation systems (ATG5, ATG7). The Class III PI(3)K-associated protein, Rubicon, has been identified as required for LAP, yet non-essential for autophagy.

Rubicon (RUN domain and cysteine-rich domain containing, Beclin 1-interacting protein) is a negative regulator of canonical autophagy through its involvement in the localization and activity of the Class III PI3K complex. Rubicon binds to Beclin 1 and VPS34 (vacuolar protein sorting 34), the catalytic subunit of the Class III PI3K complex, and the interaction between Rubicon and VPS34 inhibits VPS34 lipid kinase activity and autophogosome formation. In contrast to canonical autophagy, Rubicon is required for efficient LAP, during which Rubicon promotes PI(3)P (phosphatidylinositol 3-phosphate) formation by VPS34 to recruit the ATG5-12 and LC3-PE (LC3-phosphatidylethanolamine) conjugation systems and to stabilize and activate the NOX2 (catalytic, membrane-bound subunit of NADPH oxidase) complex. Rubicon further interacts with the p22^{phox} subunit of NOX2 to stabilize the complex for optimal ROS (reactive oxygen species) production in LAP.

In both canonical autophagy and LAP, the E3-ligase complex ATG7 and ATG10 mediates the conjugation of ubiquitin-like ATG5 to ATG12 in association with ATG16L1 to form a stabilizing, multimeric complex. Conversion of cytosolic LC3 to lipidated LC3-I is mediated by ATG4, which cleaves the LC3 precursor allowing it to be subsequently conjugated to the lipid, phosphatidylethanolamine (PE), via the activity of ATG7 and ATG3. The ATG5/12/16L1 complex is also required for the conversion of LC3I to LC3-II in canonical autophagy and LAP. However, while LC3 lipidation plays a key role in both canonical and non-canonical autophagy pathways, recent studies have shown that the WD-domain of the autophagy protein Atg16L1 is essential for single membrane lipidation of LC3, but dispensable for the canonical autophagy pathway (Fletcher et al., EMBO J 37:e97840 (2018); Fracchiolla and Martens, EMBO J 37:e98895 (2018)).

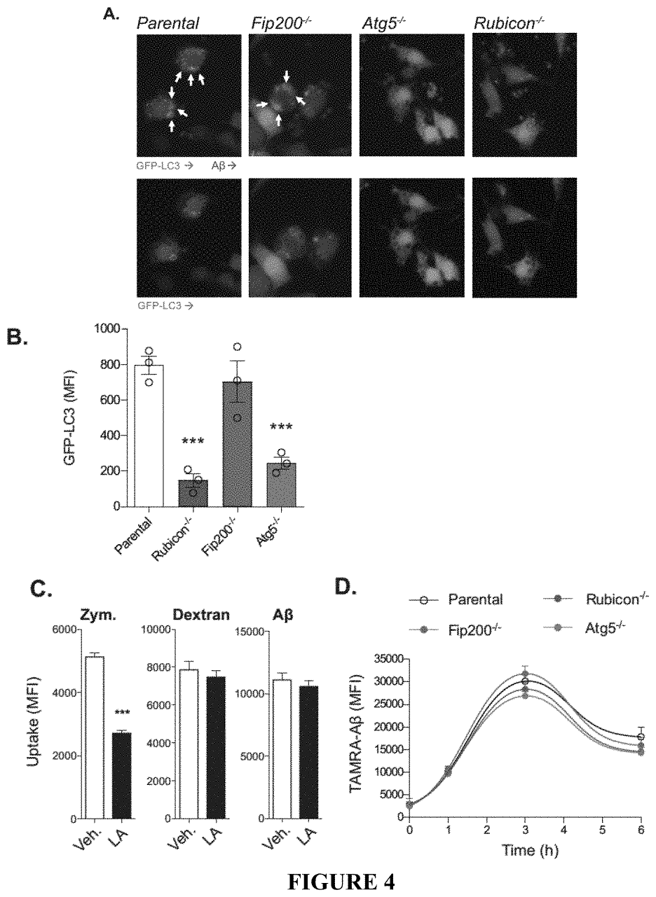

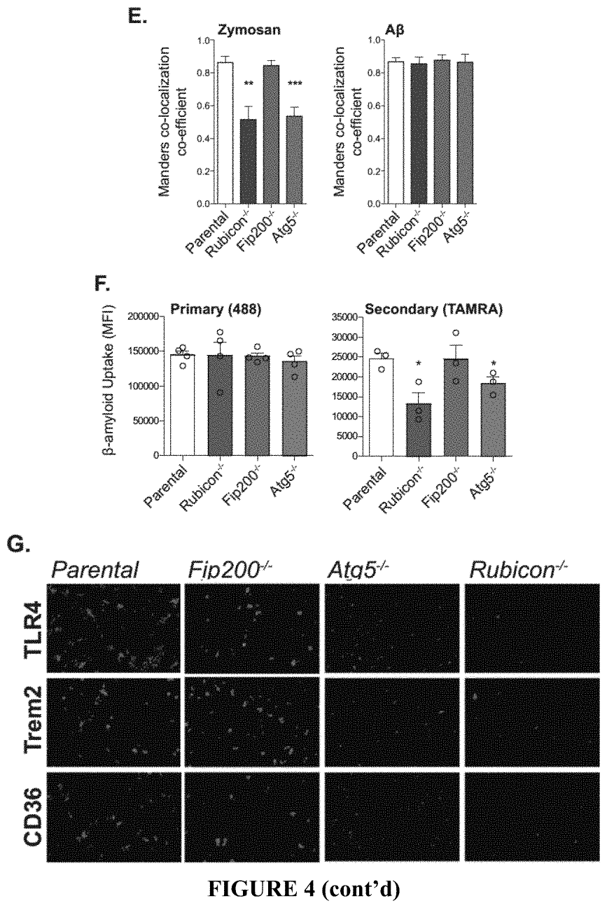

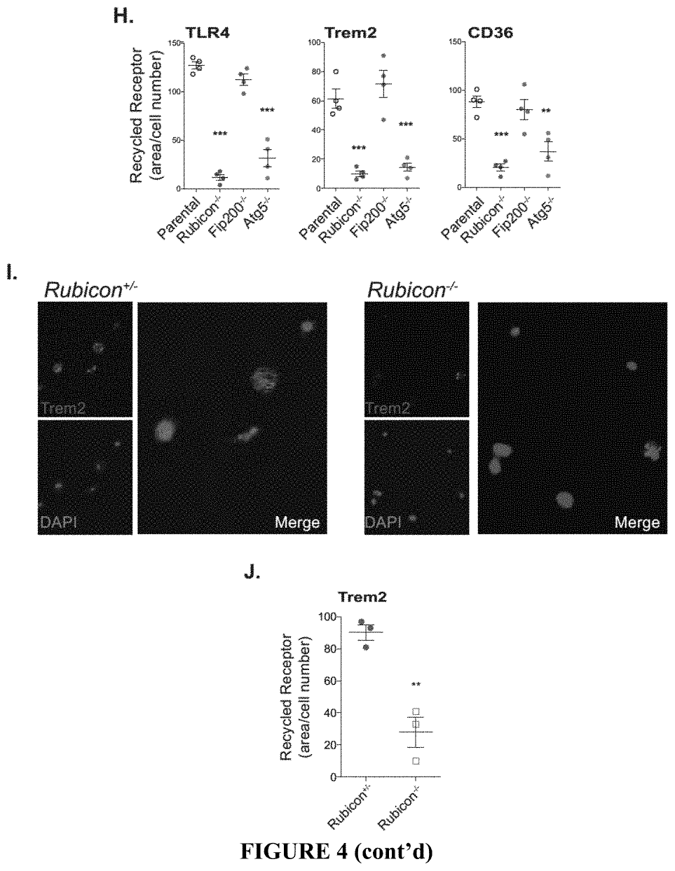

Although the LANDO pathway shares some of the same components with the canonical autophagy and LAP pathway, each of the three pathways are distinct from one another. While LAP functions to promote phagosome maturation and cargo destruction (Abnave et al. (2014) Cell Host Microbe 16:338-350; Akoumianaki et al. (2016) Cell Host Microbe 19:79-90; Cunha et al. (2018) Cell 175:429-441, e416; de Luca et al. (2014) Proc Natl Acad Sci USA 111:3526-3531; Frost et al. (2015) Mol Neurobiol 52:1135-1151; Kim et al., 2013; Kyrmizi et al. (2013) J Immunol 191:1287-1299; Lai and Devenish (2012) Cells 1:396-408; Martinez et al. (2011) Proc Natl Acad Sci USA 108:17396-17401; Martinez et al. (2016) Nature 533:115-119; Martinez et al. (2015) Nat Cell Biol 17:893-906), no effects of the deletions of LANDO pathway components, such as ATG5 or Rubicon on the rate of Aβ degradation, endosome maturation, or lysosome association were observed (FIG. 4). Because the association of LC3 at the membrane of the Aβ-containing endosome was not altered by phagocytic inhibition, this pathway is called LANDO. It was found that LANDO is required for the recycling of Aβ receptors (CD36, TREM2, and TLR4) from the internalized endosome to the plasma membrane.

Rubicon and ATG5 are required for the recycling of Aβ receptors. In addition to Rubicon and ATG5, it was further found that Beclin1, VPS34, ATG7, and ATG4 were required for the recycling of Aβ receptors, while ULK1 (Unc-51-like autophagy activating kinase), FIP200 (FAK-interacting protein of 200 kDa), and ATG14 were dispensable for this effect. The Legionella-derived protease, RavZ, which irreversibly cleaves lipidated LC3 (Choy et al. (2012) Science 338:1072-1076; Kwon et al. (2017) Autophagy 13:70-81) also prevented this receptor recycling. The role for ATG4, which processes LC3 proteins to LC3-I for lipidation, and the effect of RavZ, as well as the roles for the ligation machinery (ATG7, ATG5) strongly suggest that lipidation of LC3 at the endosome functions in the recycling of these receptors. Although the WD-domain of Atg16L1 has been identified to play a role in single membrane lipidation of LC3 (Fletcher et al., EMBO J 37:e97840 (2018); Fracchiolla and Martens, EMBO J 37:e98895 (2018)), its role in recycling of Aβ receptors remains to be explored.

Overall, LANDO is distinct from canonical autophagy and the LAP pathway and plays a requisite role in the recycling of Aβ receptors. LANDO in the myeloid compartment of the CNS functions to protect neurons from the neuroinflammatory and neurodegenerative effects of Aβ deposition.

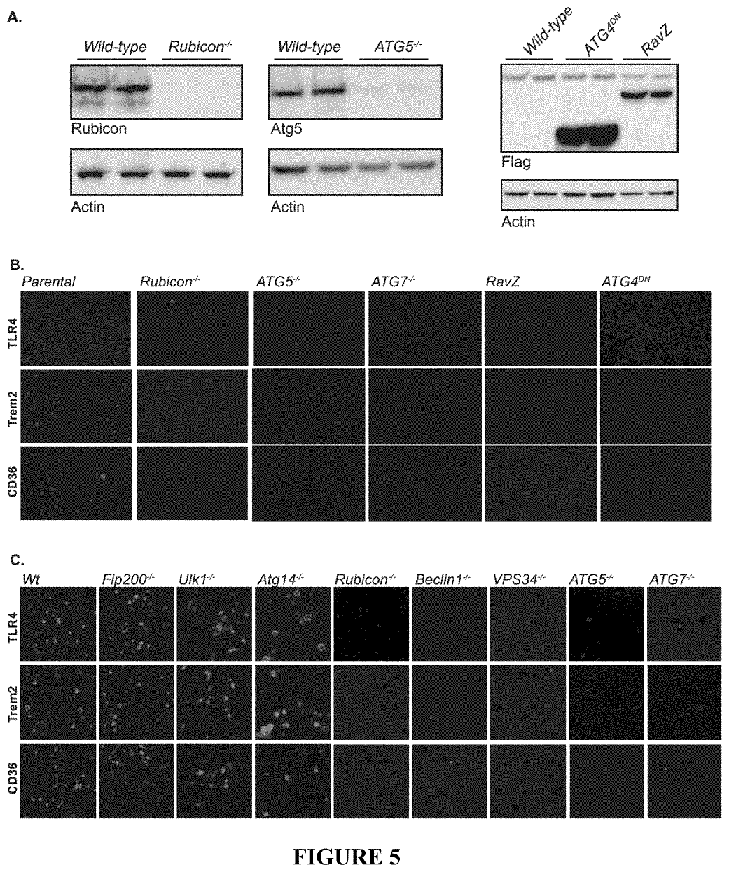

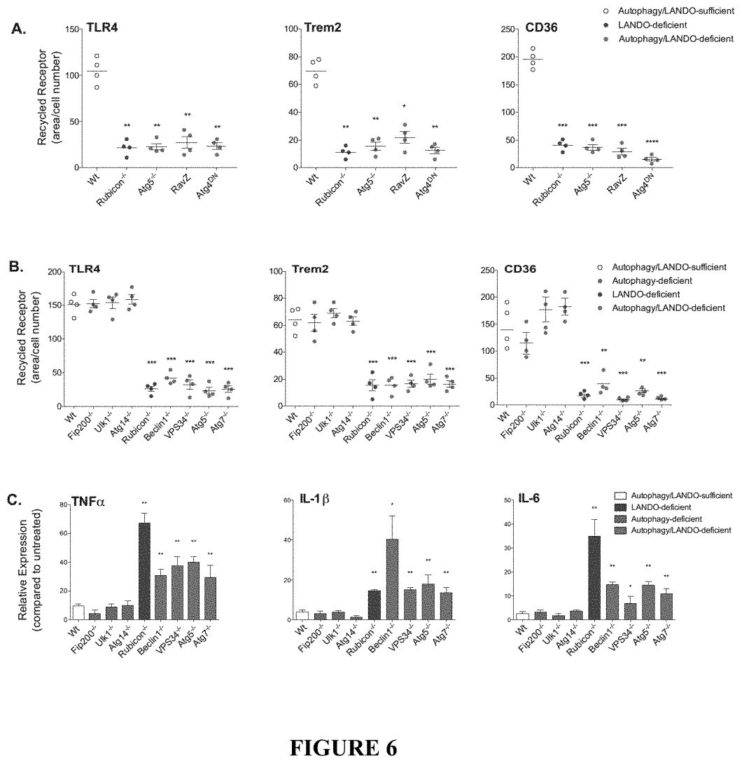

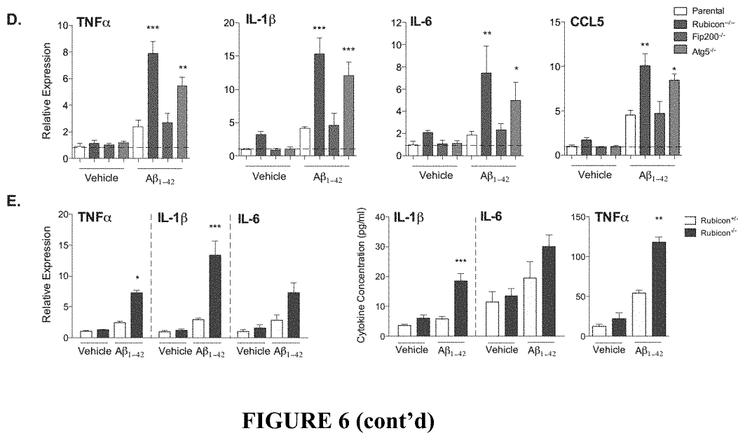

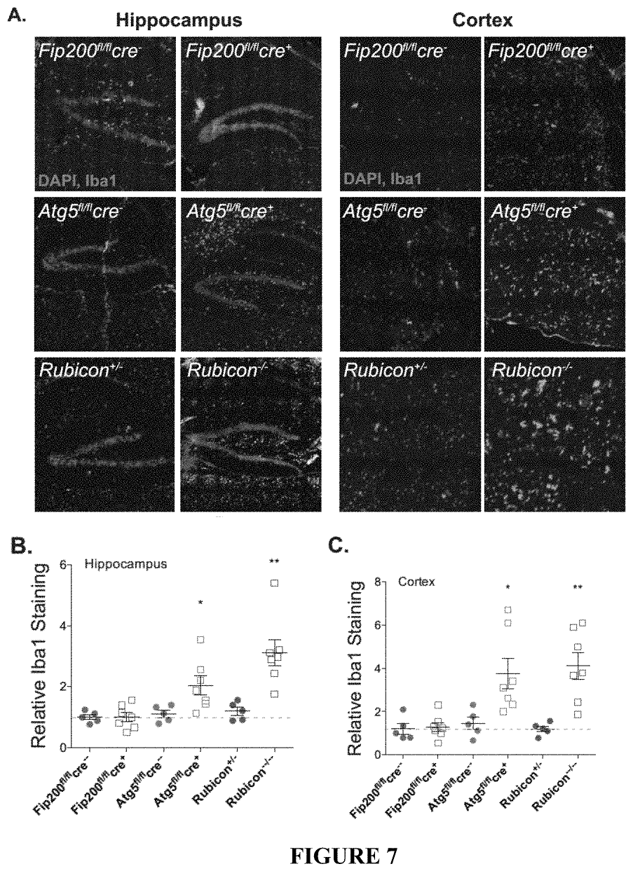

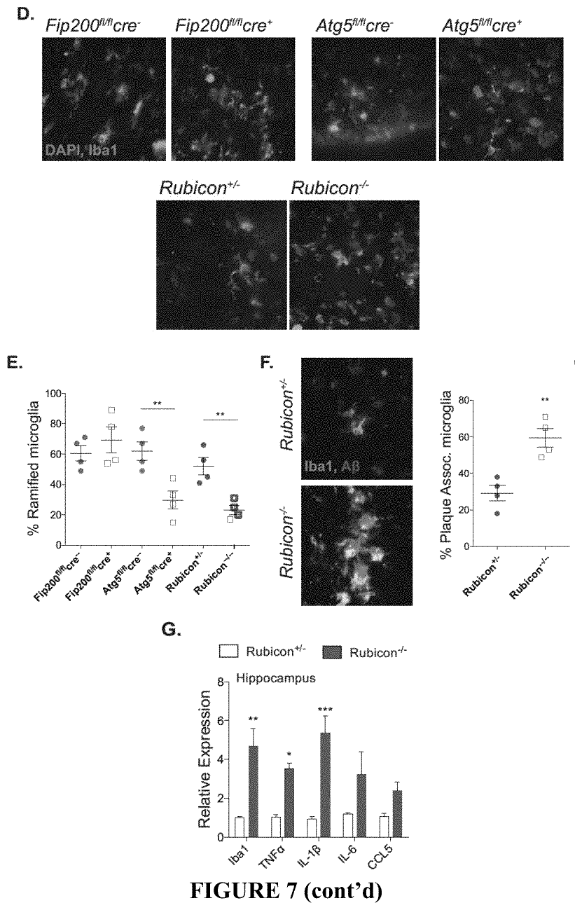

LANDO functions in microglia not only to promote Aβ clearance but also to promote an anti-inflammatory immune response. LANDO-associated proteins function to limit the expression and production of inflammatory cytokines and chemokines in response to β-amyloid in bone marrow-derived macrophages, RAW264.7 myeloid cells, BV2 microglial cells, and primary microglia in vitro (FIG. 6), and in the CNS of 5×FAD animals (FIG. 7). Loss of LANDO affects secondary uptake of Aβ and while not being bound by any theory or mechanism of action, it is believed that these Aβ deposits signal at the cell surface via other receptors to promote inflammatory signaling.

Methods are provided for clearing Aβ in a subject deficient in Aβ clearance by administering an effective amount of a pharmaceutical composition that activates or enhances the LANDO pathway. The present disclosure also provides methods for decreasing neuroinflammation or neurodegeneration in a LANDO-deficient subject by administering an effective amount of a pharmaceutical composition that activates or enhances the LANDO pathway. Also provided are methods for treating Alzheimer's disease by administering an effective amount of a pharmaceutical composition that activates or enhances the LANDO pathway to a subject diagnosed with Alzheimer's disease or demonstrating symptoms of the disease.

Methods are provided for identifying a compound that modulates LANDO activity and does not significantly modulate LAP activity, wherein the method comprises measuring a first level of LANDO activity and LAP activity in a cell or tissue, contacting the cell or tissue with a candidate compound, and measuring a second level of LANDO activity and LAP activity of the cell or tissue after contact with the candidate compound, and comparing the first and second level of LANDO and LAP activity and selecting compounds that modulate the LANDO activity and do not significantly modulate the LAP activity.

Further, a non-human animal model of neuroinflammation and neurodegeneration is provided in which the animal comprises microglial LANDO knockdown or knockout and at least one additional genetic manipulation that contributes to neuroinflammation or neurodegeneration. The non-human animal model exhibits accelerated disease pathology and neurodegeneration, reactive microgliosis, neuroinflammation, tau pathology, and behavioral impairment is observed, thereby replicating the major aspects of human disease in a rapidly developing, manipulatable animal model. Methods of making the genetically modified non-human animal model and methods for identifying a compound that modulates neuroinflammation or neurodegeneration using the animal model are provided.

### 2. Non-Human Animal Model of Neuroinflammation

The present disclosure provides a genetically modified non-human animal model of neuroinflammation or neurodegeneration, wherein the animal comprises microglial LANDO knockdown or knockout and at least one additional genetic modification that contributes to neuroinflammation or neurodegeneration.

In some embodiments, the microglial LANDO knockdown or knockout increases the penetrance of neuroinflammation or neurodegeneration associated with the additional genetic modification, reduces the age of onset of neuroinflammation or neurodegeneration, or both increases penetrance and reduces the age of onset of neuroinflammation or neurodegeneration, when compared to an animal of the same species lacking microglial LANDO knockdown or knockout, but having the genetic modification that contributes to neuroinflammation or neurodegeneration.

As used herein, the term “penetrance” refers to the extent to which a particular gene or set of genes is phenotypically expressed in individuals carrying it, which is measured by the proportion of individuals carrying this particular gene or set of genes that also express an associated trait. In particular embodiments, the LANDO knockdown or knockout increases the percentage of individuals having the additional genetic modification that also exhibit neuroinflammation or neurodegeneration. In some of these embodiments, the percentage of individuals exhibiting neuroinflammation or neurodegeneration is increased by LANDO knockdown or knockout by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200% or more.

As used herein “age of onset” refers to the age at which an individual acquires, develops, or first experiences a condition or symptoms of a disease or disorder (e.g., neuroinflammation or neurodegeneration). In some embodiments, the LANDO knockdown or knockout reduces the age of onset of neuroinflammation or neurodegeneration as compared to an animal having the genetic modification associated with neuroinflammation or neurodegeneration. In some of these embodiments, the age of onset of neuroinflammation or neurodegeneration is reduced by LANDO knockdown or knockout by days, weeks, months, or years, including 5 days, 10 days, 20 days, 1 month, 2 months, 3 months, 4 months, 5 months, 6 months, 7 months, 8 months, 9 months, 10 months, 11 months, 1 year, 1.25 years, 1.5 years, 1.75 years, 2 years, 2.25 years, 2.5 years, 2.75 years, 3 years.

Given the increased penetrance and/or reduced age of onset of neuroinflammation or neurodegeneration, the presently disclosed mouse models are particularly useful in studying neuroinflammation, neurodegeneration, behavioral and/or memory impairment resulting from neurodegeneration, RA clearance and/or deposition, tau hyperphosphorylation, the LANDO pathway, Alzheimer's disease, and screening for compounds that modulate the LANDO pathway, and can be used to treat neuroinflammation or neurodegeneration in general, or Alzheimer's disease in particular.

As used herein, the term “knockout” refers to a method by which at least one of an organism's genes is made inoperative. As used herein, the term “knockout” refers to either a heterozygous knockout, wherein only one of two gene copies (alleles) is made inoperative, or a homozygous knockout in which both copies of a gene are made inoperative. The term “knockout” can also encompass a knockin, in those situations wherein a gene is replaced with another gene. The term “knockdown” refers to a method by which the expression of one or more of an organisms's genes is reduced. A LANDO knockdown or knockout refers to a knockdown or knockout of a LANDO-related molecule. In particular embodiments, the microglial LANDO knockdown or knockout targets Rubicon, ATG5, or both. Additionally, or alternatively, LANDO knockdown or knockout may target the WD-domain of Atg16L.

Any method known in the art to knockout or knockdown a LANDO-related molecule can be used, such as RNA interference, or homologous recombination with or without the use of a site-specific nuclease (e.g., zinc finger nucleases, CRISPR-cas nucleases, TALENs, meganucleases).

As many components of LANDO are also involved in autophagy or LAP and might be required for development or post-natal survival, in some embodiments, the LANDO knockout or knockdown is tissue-specific. In some of those embodiments wherein the LANDO knockout or knockdown is tissue-specific, the knockout or knockdown is specific to cells of the myeloid lineage and microglia. Promoters that are specific for cells of the myeloid lineage and microglia are known in the art. Non-limiting examples of such a promoter are the lysozyme 2 promoter, the chemokine receptor CX3CR1 promoter (Yona et al. (2013) Immunity 38(1):79-91), and the transmembrane protein 119 (TMEM119) promoter (The Jackson Laboratory).

In some of those embodiments wherein the LANDO knockout or knockdown is tissue-specific, a site-specific recombinase system is used, wherein expression of the site-specific recombinase is under the control of a tissue-specific promoter. As used herein, a “site-specific recombinase system” refers to both a site-specific recombinase that performs rearrangements of DNA segments by recognizing and binding to short DNA sequences (recombination sites), at which the recombinase cleaves the DNA backbone and exchanges the two DNA helices involved and rejoins the DNA strands, along with the recombination sites. The site-specific recombinase system can be used to generate excisions, inversions, or insertions of replacement DNA. Site-specific recombinase systems are known in the art and include, but are not limited to, the Cre-lox system and the FLP-frt system. In particular embodiments, a tissue-specific promoter (e.g., the lysozyme M promoter) regulates the expression of the site-specific recombinase (e.g., Cre).

The presently disclosed genetically modified non-human animal models comprise a genetic modification that contributes to neuroinflammation or neurodegeneration, in addition to the LANDO knockout or knockdown. Any genetic modification, such as a genomic or somatic mutation (e.g., deletion, addition, substitution) or transgene expression, known in the art to cause neuroinflammation or neurodegeneration may be used. In some embodiments, however, this additional genetic modification comprises modifications that lead to the overexpression of amyloid precursor protein (APP) or the aggregation of RA. In some of these embodiments, the non-human animal transgenically expresses APP. In particular embodiments, the non-human animal transgenically expresses APP comprising at least one mutation present in familial Alzheimer's disease (FAD). In some of these embodiments, the non-human animal model transgenically expresses a mutated APP comprising at least one of K670N, M671L, I716V, and V717I in relation to the human 770 amino acid APP (NCBI NP_000475.1). In particular embodiments, the non-human animal model transgenically expresses a mutated APP comprising all of the following mutations: K670N, M671L, I716V, and V717I in relation to the human 770 amino acid APP.

In some embodiments, the non-human animal model transgenically expresses mutant human presinilin 1 comprising at least one of M146L and L286V mutations in relation to human presinilin 1 (NCBI NP_000012.1). In some of these embodiments, the transgenic expression of mutant human APP and/or presinilin 1 is under the control of a tissue-specific promoter that is expressed in the central nervous system. Suitable promoters are known in the art and include, but are not limited to, the Thy1 promoter, the platelet derived growth factor (PDGF) promoter (Games et al. (1995) Nature 373(6514):523-527), and the prion protein (PrP) promoter (Hsiao et al. (1996) Science 274(5284):99-102).

In particular embodiments, the non-human animal model comprises a 5×FAD transgenic animal transgenically expressing a mutant human APP with each of the following mutations: K670N, M671L, I716V, and V717I, and transgenically expressing a mutant human presinilin 1 comprising a M146L mutation and a L286V mutation.

In alternative embodiments, the non-human animal model comprises a deletion or mutation of the WD-domain of Atg16L (Atg16L^{ΔWD}), which is also referred to herein as the Atg16L WD-domain. In some embodiments, the non-human animal model comprises an Atg16L WD-domain deficient (Atg16L^{ΔWD}) animal transgenically expressing a mutant human APP with at least one of, or all of, the following mutations: K670N, M671L, I716V, and V717I, and transgenically expressing a mutant human presinilin 1 comprising a M146L mutation and a L286V mutation.

Any non-human animal may be genetically modified according to the subject disclosure. Nonlimiting examples include laboratory animals, domestic animals, livestock, etc., e.g., species such as murine, rodent, canine, feline, porcine, equine, bovine, ovine, non-human primates, etc.; for example, mice, rats, rabbits, hamsters, guinea pigs, cattle, pigs, sheep, goats and other transgenic animal species, particularly-mammalian species, as known in the art. In other embodiments, the non-human animal may be a bird, e.g., of Galliformes order, such as a chicken, a turkey, a quail, a pheasant, or a partridge; e.g., of Anseriformes order, such as a duck, a goose, or a swan, e.g., of Columbiformes order, such as a pigeon or a dove. In various embodiments, the subject genetically modified animal is a mouse, a rat or a rabbit.

In some embodiments, the non-human animal is a mammal. In some such embodiments, the non-human animal is a small mammal, e.g., of the superfamily Dipodoidea or Muroidea. In one embodiment, the genetically modified animal is a rodent. In one embodiment, the rodent is selected from a mouse, a rat, and a hamster. In one embodiment, the rodent is selected from the superfamily Muroidea. In one embodiment, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rats, bamboo rats, and zokors). In a specific embodiment, the genetically modified rodent is selected from a true mouse or rat (family Muridae), a gerbil, a spiny mouse, and a crested rat.

In one embodiment, the subject genetically modified non-human animal is a mouse, e.g. a mouse of a C57BL strain (e.g. C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C57BL/Ola, etc.); a mouse of the 129 strain (e.g. 129P1, 129P2, 129P3, 129X1, 129S1 (e.g., 12951/SV, 12951/SvIm), 12952, 129S4, 129S5, 12959/SvEvH, 129S6 (129/SvEvTac), 129S7, 129S8, 129T1, 129T2); a mouse of the BALB strain; e.g., BALB/c; and the like. See, e.g., Festing et al. (1999) Mammalian Genome 10:836, see also, Auerbach et al (2000) Establishment and Chimera Analysis of 129/SvEv- and C57BL/6-Derived Mouse Embryonic Stem Cell Lines). In another embodiment, a mouse is a mix of the aforementioned strains.

In another embodiment, the subject genetically modified non-human animal is a rat. In one such embodiment, the rat is selected from a Wistar rat, an LEA strain, a Sprague Dawley strain, a Fischer strain, F344, F6, and Dark Agouti. In another embodiment, the rat strain is a mix of two or more strains selected from the group consisting of Wistar, LEA, Sprague Dawley, Fischer, F344, F6, and Dark Agouti.

Any method known in the art for generating the non-human animal model can be used. Such techniques are well-known in the art and include, but are not limited to, pronuclear microinjection, transformation of embryonic stem cells, homologous recombination and knock-in techniques. Methods for generating genetically modified animals that can be used include, but are not limited to, those described in Sundberg and Ichiki (2006) Genetically Engineered Mice Handbook, CRC Press; Hofker and van Deursen (2002) Genetically modified Mouse Methods and Protocols, Humana Press; Joyner (2000) Gene Targeting: A Practical Approach, Oxford University Press; Turksen (2002) Embryonic stem cells: Methods and Protocols in Methods Mol Biol., Humana Press; Meyer et al. (2010) Proc. Nat. Acad. Sci. USA 107:15022-15026; and Gibson (2004), A Primer of Genome Science 2nd ed. Sunderland, Mass.: Sinauer; U.S. Pat. No. 6,586,251; Rathinam et al. (2011) Blood 118:3119-28), Willinger et al. (2011) Proc Natl Acad Sci USA 108:2390-2395; Rongvaux et al. (2011) Proc Natl Acad Sci USA 108:2378-83; and Valenzuela et al. (2003) Nat Biot 21:652-659.

For example, the subject genetically modified animals can be created by introducing the nucleic acid construct into an oocyte, e.g., by microinjection, and allowing the oocyte to develop in a female foster animal. In preferred embodiments, the nucleic acid construct is injected into fertilized oocytes. Fertilized oocytes can be collected from superovulated females the day after mating and injected with the expression construct. The injected oocytes are either cultured overnight or transferred directly into oviducts of 0.5-day p.c. pseudopregnant females. Methods for superovulation, harvesting of oocytes, expression construct injection and embryo transfer are known in the art and described in Manipulating the Mouse Embryo (2002) A Laboratory Manual, 3rd edition, Cold Spring Harbor Laboratory Press. Offspring can be evaluated for the presence of the introduced nucleic acid by DNA analysis (e.g., PCR, Southern blot, DNA sequencing, etc.) or by protein analysis (e.g., ELISA, Western blot, etc.).

As another example, the nucleic acid construct may be transfected into stem cells (e.g., ES cells or iPS cells) using well-known methods, such as electroporation, calcium-phosphate precipitation, lipofection, etc. The cells can be evaluated for the presence of the introduced nucleic acid construct by DNA analysis (e.g., PCR, Southern blot, DNA sequencing, etc.) or by protein analysis (e.g., ELISA, Western blot, etc.). Cells determined to have incorporated the expression construct can then be introduced into preimplantation embryos. For a detailed description of methods known in the art useful for the compositions and methods of the invention, see Nagy et al., (2002, Manipulating the Mouse Embryo: A Laboratory Manual, 3rd edition, Cold Spring Harbor Laboratory Press), Nagy et al. (1990, Development 110:815-821), U.S. Pat. Nos. 7,576,259, 7,659,442, 7,294,754, and Kraus et al. (2010, Genesis 48:394-399).

Genetically modified LANDO knockdown or knockout animals can be bred to additional animals carrying the genetic modification in order to produce a non-human animal that is homozygous for the LANDO knockdown or knockout.

In certain embodiments, the method comprises knocking down or knocking out LANDO in microglial tissues in a non-human animal comprising at least one additional genetic modification that contributes to neuroinflammation or neurodegeneration. The method can comprise simply crossing a non-human animal comprising the microglial LANDO knockdown or knockout with another non-human animal of the same species that comprises the at least one additional genetic modification. Alternatively, the method comprises knocking down or knocking out a LANDO-related molecule in a non-human animal already comprising the additional genetic modification.

In particular embodiments, the methods for making the non-human model further comprise introducing the at least one additional genetic modification that contributes to neuroinflammation or neurodegeneration into the non-human animal.

### 3. Methods of Treatment

Methods and compositions are provided herein for decreasing neuroinflammation or neurodegeneration in a subject comprising administration of an effective amount of a pharmaceutical composition that activates or enhances the LANDO pathway. In certain embodiments the subject to be treated is a LANDO-deficient subject, a subject with neuroinflammation, a subject with neurodegeneration, a subject with impaired β-amyloid clearance, a subject with β-amyloid accumulation, a subject with reactive microgliosis, a subject with hyperphosphorylation of tau, and/or a subject with behavioral and memory deficits when compared to an appropriate control.

In certain embodiments, administration of an effective amount of a pharmaceutical composition that targets the LANDO pathway can decrease the symptoms of LANDO-deficiency, decrease neuroinflammation and/or neurodegeneration, or increase β-amyloid clearance in a subject. “Treatment” is herein defined as curing, healing, alleviating, relieving, altering, remedying, ameliorating, improving, or affecting the condition or the symptoms of a LANDO-deficient subject. The subject to be treated can be suffering from or at risk of developing a neuroinflammatory or neurodegenerative disease or be at risk of developing any disease associated with LANDO-deficiency. Reducing at least one symptom of a LANDO-deficiency, neuroinflammation, neurodegeneration, Alzheimer's disease, or decreased β-amyloid clearance refers to a statistically significant reduction of at least one symptom. Such decreases or reductions can include, for example, at least a 5%, 10%, 20%, 30%, 40%, 50%, 60%, 70%, 80%, 90%, 95%, or 100% decrease in the measured or observed level of at least one symptom, as disclosed elsewhere herein. Non-limiting examples of symptoms of LANDO-deficiency, neuroinflammation, neurodegeneration, Alzheimer's disease, or decreased β-amyloid clearance include behavioral and memory deficits, mood changes, anxiety, agitation, loss of inhibition, seizures, cognitive deficits, and personality changes.

In some embodiments, the subject is a LANDO-deficient subject having reduced expression of a LANDO-related molecule. As used herein, the term “LANDO-related” refers to any nucleic acid, protein, cytokine, or any other molecule that participates in the LANDO pathway. LANDO-related molecules include, but are not limited to Beclin1, ATG7, ATG5, ATG4, LC3, Rubicon, and VPS34.

As used herein, the term “reduced” refers to any reduction in the expression or activity of a LANDO-related molecule when compared to the corresponding expression or activity of the same LANDO-related molecule in a control cell. Such a reduction may be up to 5%, 10%, 20%, 30%, 40%, 50%, 60%, 70%, 80%, 90%, 95%, or up to 100%. Accordingly, the term “reduced” encompasses both a partial knockdown and a complete knockdown of the activity of a LANDO-related molecule.

By “subject” is intended animals. In specific embodiments, subjects are mammals, e.g., primates or humans. In other embodiments, subjects include domestic animals, such as a feline or canine, or agricultural animals, such as a ruminant, horse, swine, poultry, or sheep. In specific embodiments, the subject undergoing treatment with the pharmaceutical formulations of the invention is a human. In some embodiments, the human undergoing treatment can be a newborn, infant, toddler, preadolescent, adolescent or adult. The subjects of the invention may be suffering from the symptoms of a neuroinflammatory or neurodegenerative disorder or may be at risk for developing a neuroinflammatory or neurodegenerative disorder.

The expression of three proteins obligatory for LANDO function, Beclin1, ATG5, and ATG7, decreases with age (Lipinski et al. (2010) Proc Natl Acad Sci USA 107:14164-14169), which may lead to an insufficiency in LANDO, establishing a putative risk factor for physiological to pathological transition. Accordingly, the presently disclosed compositions can be administered to subjects that are LANDO-deficient. The term “LANDO-deficient” refers to an alteration in the LANDO pathway such that the LANDO pathway does not function properly. That is, a LANDO-deficient organism does not effectively recycle β-amyloid receptors back to the plasma membrane and subsequently suffers from β-amyloid deposits and increased neuroinflammation and neurodegeneration. A LANDO-deficient subject could have an increase or decrease in the expression or activity of any LANDO related molecule (e.g., Rubicon, ATG5, Beclin1, VPS34, ATG7, and ATG4), or a defect in the subject's clearance of β-amyloid. Particularly, a LANDO-deficient subject has an increase in pro-inflammatory cytokines or a decrease in anti-inflammatory cytokines, which may lead to increased inflammation, increased levels of neurotoxic β-amyloid accumulation, which can lead to reactive microgliosis, hyperphosphorylation of tau, neurodegeneration, and behavioral and memory deficits.

The methods and compositions disclosed herein involve a method for decreasing neuroinflammation or neurodegeneration, for treating Alzheimer's disease, or for clearing βA by administering to a subject in need thereof an effective amount of a pharmaceutical composition that activates or enhances the LANDO pathway. Such compositions can be identified using the screening methods disclosed herein. In one non-limiting embodiment, the method for decreasing neuroinflammation or neurodegeneration, for treating Alzheimer's disease, or for clearing βA comprises administering an effective amount of an agent which increases or enhances the biological activity of Rubicon. In another non-limiting embodiment, the method for decreasing neuroinflammation or neurodegeneration, for treating Alzheimer's disease, or for clearing βA comprises administering an effective amount of an agent which increases or enhances the biological activity of ATG5. In another non-limiting embodiment, the method for decreasing neuroinflammation or neurodegeneration, for treating Alzheimer's disease, or for clearing βA comprises administering an effective amount of an agent which increases or enhances the biological activity of the WD-domain of Atg16L.

A subject is considered successfully treated if they exhibit, for example, an improvement in any one of the symptoms of neuroinflammation, neurodegeneration, LANDO deficiency, Alzheimer's disease, or decreased βA clearance.

In some embodiments, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. A BBB disrupting agent can be co-administered with the agent when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversibly affixed (see e.g. US Application Nos. 20080081064 and 20090196903, incorporated herein by reference).

A. Neuroinflammatory or Neurodegenerative Disease

In some embodiments, neuroinflammatory disorders associated with a LANDO deficiency can be treated or prevented. Neuroinflammatory diseases can arise where there is an inflammation of the brain or neuronal tissue. The term “neuroinflammatory diseases” as used herein, includes, but are not limited to, local inflammatory responses and systemic inflammation.

In some embodiments, neurodegenerative disorders associated with a LANDO deficiency can be treated or prevented. The term “neurodegenerative disorders” as used herein, refers to the progressive loss of the structure or function of neurons, including the death of neurons. Some disorders have hallmarks of both neuroinflammation and neurodegeneration.

In specific embodiments, the disorder to be treated by the methods and compositions described herein is Alzheimer's disease. Further disorders that could be treated or prevented by the methods and compositions described herein include, but are not limited to CNS inflammatory disorders, Parkinson's disease, multiple sclerosis, Huntington's disease, and amyotrophic lateral sclerosis.

In some embodiments, administration of an effective amount of a pharmaceutical composition that activates or enhances the LANDO pathway results in a decrease in pro-inflammatory cytokine production, which may decrease or prevent an inflammatory response. As used herein, a decrease in the level of pro-inflammatory cytokine production comprises any statistically significant decrease in the level of pro-inflammatory cytokine production in a subject when compared to an appropriate control. Such decreases can include, for example, at least a 5%, 10%, 20%, 30%, 40%, 50%, 60%, 70%, 80%, 90%, or 100% decrease in the level of proinflammatory cytokines. Non-limiting examples of proinflammatory cytokines include IL1-alpha, IL1-beta, TNF-alpha, IL-2, IL-3, IL-6, IL-7, IL-9, IL-12, IL-17, IL-18, TNF-alpha, CCL5, LT, LIF, IFN-alpha, IFN-beta, or IFN-gamma. Methods to assay for cytokine levels are known and include, for example Leng S., et al. (2008) J Gerontol A Biol Sci Med Sci 63(8): 879-884. Methods to assay for the production of pro-inflammatory cytokines include multiplex bead assay, ELISPOT and flow cytometry. See, for example, Maecker et al. (2005) BMC Immunology 6:13.

In certain embodiments, the administration of an effective amount of a pharmaceutical composition that activates or enhances the LANDO pathway results in an increase in anti-inflammatory cytokine production. As used herein, an “increase in” or “increasing” anti-inflammatory cytokine production comprises any statistically significant increase the anti-inflammatory cytokine level when compared to an appropriate control. Such increases can include, for example, at least a 5%, 10%, 20%, 30%, 40%, 50%, 60%, 70%, 80%, 90%, 100%, 150%, 200% or greater increase in the anti-inflammatory cytokine level. Such increases can also include, for example, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater increase in the anti-inflammatory cytokine level. Anti-inflammatory cytokines of the invention include interleukin (IL)-1 receptor antagonist, IL-4, IL-10, IL-11, and IL-13, IL-16, IFN-alpha, TGF-beta, G-CSF. Methods to assay for the level of anti-inflammatory cytokine level, are known. See, for example, Leng S., et al. (2008) J Gerontol A Biol Sci Med Sci 63(8): 879-884. Methods to assay for the production of anti-inflammatory cytokines include multiplex bead assay, ELISPOT and flow cytometry. See, for example, Maecker et al. (2005) BMC Immunology 6:13.

Inflammatory cytokine production can also be measured by assaying the ratio of anti-inflammatory cytokine production to proinflammatory cytokine production. In specific aspects, the ratio of anti-inflammatory cytokine production to proinflammatory cytokine production is increased by about 1, 2, 3, 4, 5, 6, 7, 8, 9, 10, 20, 30, 40, 50, 60, 70, 80, 90, 100, 300, 600, 900, 1000 fold or greater when compared to an appropriate control. In other aspects, the ratio of anti-inflammatory cytokine production to pro-inflammatory cytokine production is increased by about 1 to 5 fold, about 5 to 10 fold, about 10 to 20 fold, about 20 to 30 fold, about 30 to 40 fold, about 40 fold to 60 fold, about 60 fold to 80 fold, about 80 fold to about 100 fold, about 100 to 200 fold, about 200 fold to 300 fold, about 300 to 400 fold, about 400 to about 500 fold, about 500 to about 500 fold, about 500 fold to about 700 fold, about 700 fold to 800 fold, about 800 fold to about 1000 fold or greater when compared to an appropriate control. Methods to determine the ratio of anti-inflammatory cytokine production to pro-inflammatory cytokine production can be found, for example, Leng S., et al. (2008) J Gerontol A Biol Sci Med Sci 63(8): 879-884. Methods to assay for the production of cytokines include multiplex bead assay, ELISPOT and flow cytometry. See, for example, Maecker et al. (2005) BMC Immunology 6:13.

B. Alzheimer's Disease

In certain embodiments, administration of an effective amount of a pharmaceutical composition that activates or enhances the LANDO pathway treats Alzheimer's Disease in a subject diagnosed with Alzheimer's disease or demonstrating symptoms of the disease or those at increased risk for developing the disease.

Alzheimer's disease (AD) is a multifactorial progressive neurodegenerative disorder characterized by loss of memory and cognitive deficits. Alzheimer's disease is the most common form of both senile and presenile dementia in the world and is recognized clinically as relentlessly progressive dementia that presents with increasing loss of memory, intellectual function and disturbances in speech (Merritt (1979) A Textbook of Neurology, 6th edition, pp. 484-489 Lea & Febiger, Philadelphia). The disease itself usually has a slow and insidious progress that affects both sexes equally, worldwide. It begins with mildly inappropriate behavior, uncritical statements, irritability, a tendency towards grandiosity, euphoria and deteriorating performance at work; it progresses through deterioration in operational judgement, loss of insight, depression and loss of recent memory; it ends in severe disorientation and confusion, apraxia of gait, generalized rigidity and incontinence (Gilroy & Meyer (1979) Medical Neurology, pp. 175-179 MacMillan Publishing Co.).

Alzheimer's disease afflicts an estimated 4 million human beings in the United States alone at a cost of 35 billion dollars a year (Hay &: Ernst (1987) Am. J. Public Health 77:1169-1175). It is found in 10% of the population over the age of 65 and 47% of the population over the age of 85 (Evans et al. (1989) JAMA, 262:2551-2556). A very small percentage of AD cases (5-7%) arise in family clusters (“familial” AD) with early onset (before the age of 60) and the remaining majority of cases (>90%) with late onset (after the age of 60). Current state of knowledge suggests that familial AD is caused by a dominant mutation in one of three genes: presinilin 1 (PSEN1), presinilin 2 (PSEN2), or amyloid precursor protein (APP). A person with one of these mutations is at risk of developing symptoms before age 60. Such families are quite rare, but the 50 percent risk for each child of an affected member to carry the causative mutation means that these tests can be important for those at risk.

Genetic factors have also been associated with the sporadic or non-familial form of the disease and the allele e4 of the apolipoprotein E (Apo E) significantly increases the risk of AD, but is neither necessary nor sufficient for the development of the disease. Therefore, other genetic and environmental factors are likely to be implicated and are actively being investigated.

There are several current methods used in diagnosing Alzheimer's that include neurological exam, mental status tests, mood assessment, family history, and brain imaging. Brain imaging, such as magnetic resonance imaging (MRI), computed tomography (CT), positron emission tomography (PET), can be used to rule out other diagnoses and in some cases, help to diagnose Alzheimer's disease. PET imaging can be performed in which ligands that selectively bind to amyloid-beta plaques or hyperphosphorylated tau deposits are employed. In another technique, magnetic resonance imaging (MRI) biomarkers may be detected as an indication of Alzheimer's. These include the reduction of brain volume, specifically hippocampal volume which controls the memory part of the brain. Another indication may be decreased concentrations of Aβ or increased hyperphosphorylated tau in the cerebral spinal fluid (CSF) of an individual.

Deficiencies in the LANDO pathway can result in the failure of Aβ clearance, an increase in pro-inflammatory cytokine production, reactive microgliosis, hyperphosphorylation of tau, neurodegeneration, impaired neuronal signaling leading to behavioral and memory deficits—many of the hallmarks of Alzheimer's disease. Thus, the administration of a composition that enhances or activates the LANDO pathway can be used to restore the function of the pathway and decrease symptoms of Alzheimer's disease. Any symptom of Alzheimer's disease as described herein can be reduced by the methods described herein. In a particular embodiment, inflammation is reduced by administration of an effective amount of a pharmaceutical composition that activates or enhances the LANDO pathway in a subject experiencing AD symptoms.

C. β-Amyloid Clearance

In specific embodiments, administration of an effective amount of a pharmaceutical composition that activates or enhances the LANDO pathway decreases the symptoms of a deficiency in Aβ clearance. Accordingly, administration of an effective amount of a pharmaceutical composition that activates or enhances the LANDO pathway can increase Aβ clearance. In certain embodiments, clearance of Aβ is increased because of a restoration of all or a portion of the LANDO pathway.

In some of these embodiments, the subject to which the pharmaceutical composition is administered is a LANDO-deficient subject. The LANDO-deficient subject may have reduced expression of at least one of Beclin 1, VPS34, ATG5, ATG7, ATG4, LC3, Rubicon, and Atg16L (e.g., WD-domain of Atg16L) when compared to a control subject. The subject may comprise Aβ accumulation in the cortex, hippocampus, or both and exhibit symptoms of the same. The symptoms associated with Aβ accumulation are similar to those associated with Alzheimer's disease and include behavioral and memory deficits, mood changes, anxiety, agitation, loss of inhibition, seizures, cognitive deficits, and personality changes.

Methods for measuring Aβ clearance are known in the art and are disclosed elsewhere herein. Non-limiting examples include measuring levels of Aβ in the CSF, PET imaging with ligands that selectively bind to Aβ plaques, measuring uptake of labeled Aβ in primary microglial cells isolated from the subject, and measuring recycling of Aβ receptors (e.g., TREM2, CD36, TLR4) in primary microglial cells isolated from the subject.

### 4. Pharmaceutical Compositions

The methods and compositions disclosed herein encompass administration of an effective amount of a pharmaceutical composition that enhances or activates the LANDO pathway. Methods are also disclosed herein for screening for compositions or molecules that modulate (i.e., increases or decreases) LANDO activity. As used herein, the term “specifically” means the ability of a molecule that modulates the LANDO pathway to increase or decrease LANDO activity without impacting other related processes (i.e., LAP pathway, canonical autophagy). A molecule that modulates the LANDO pathway preferentially, increases or decreases LANDO activity, but might impact other phagocytosis-related pathways. Accordingly, a molecule that modulates the LANDO pathway could be any LANDO-related nucleic acid, protein, or cytokine, such as Beclin1, VPS34, ATG5, ATG7, ATG4, LC3, Rubicon, and Atg16L (e.g., WD-domain of Atg16L).

The pharmaceutical composition may be a liquid formulation or a solid formulation. When the pharmaceutical composition is a solid formulation it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip or a film. When the pharmaceutical composition is a liquid formulation it may be formulated as an oral solution, a suspension, an emulsion or syrup. Said composition may further comprise a carrier material independently selected from, but not limited to, the group consisting of lactic acid fermented foods, fermented dairy products, resistant starch, dietary fibers, carbohydrates, proteins, and glycosylated proteins. As used herein, the pharmaceutical composition could be formulated as a food composition, a dietary supplement, a functional food, a medical food, or a nutritional product as long as the required effect is achieved.

The pharmaceutical composition according to the invention, used according to the invention or produced according to the invention may also comprise other substances, such as an inert vehicle, or pharmaceutical acceptable adjuvants, carriers, preservatives etc., which are well known. By “therapeutically effective dose,” “therapeutically effective amount,” or “effective amount” is intended an amount of the composition or molecule that enhances or activates the LANDO pathway that brings about a positive therapeutic response with respect to treatment or prevention. “Positive therapeutic response” refers to, for example, improving the condition of at least one of the symptoms of a neuroinflammatory disorder, neurodegenerative disorder, decreasing at least one symptom of Alzheimer's disease and/or increasing Aβ clearance.

Examples of possible routes of administration include parenteral, (e.g., intravenous (IV), intramuscular (IM), intradermal, subcutaneous (SC), or infusion) administration. Moreover, the administration may be by continuous infusion or by single or multiple boluses. In specific embodiments, one or both of the agents is infused over a period of less than about 4 hours, 3 hours, 2 hours or 1 hour. In still other embodiments, the infusion occurs slowly at first and then is increased over time.

Generally, the dosage of the composition that activates or enhances the LANDO pathway will vary depending upon such factors as the patient's age, weight, height, sex, general medical condition and previous medical history. In specific embodiments, it may be desirable to administer the composition that enhances or activates the LANDO pathway in the range of from about 1 to 100 mg/kg, 20 to 30 mg/kg, 30 to 40 mg/kg, 40 to 50 mg/kg, 50 to 60 mg/kg, 60 to 70 mg/kg, 70 to 80 mg/kg, 80 to 100 mg/kg, 5 to 10 mg/kg, 2 to 10 mg/kg, 10 to 20 mg/kg, 5 to 15 mg/kg, 1 to 10 mg/kg, 1 to 5 mg/kg, 2 to 5 mg/kg or any range in between 1 and 100 mg/kg.

In some embodiments of the invention, the method comprises administration of multiple doses of the composition that enhances or activates the LANDO pathway. The method may comprise administration of 1, 2, 3, 4, 5, 6, 7, 8, 9, 10, 15, 20, 25, 30, 35, 40, or more therapeutically effective doses of a composition that enhances or activates the LANDO pathway. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to improve the condition of at least one of the symptoms of a neuroinflammatory disorder, a neurodegenerative disorder, decrease at least one symptom of Alzheimer's disease, and/or increase Aβ clearance. Changes in dosage may result and become apparent from the results of diagnostic assays for detecting neuroinflammation, neurodegeneration, Alzheimer's disease symptoms, and Aβ clearance known in the art and described herein.

### 5. Methods of Identifying Compounds for Modulation of LANDO Activity, Neuroinflammation, and Neurodegeneration

The methods and compositions disclosed herein include methods for identifying a molecule or composition that modulates LANDO activity. Modulating LANDO activity refers to increasing or decreasing LANDO activity or LANDO-related neuroinflammation or LANDO-related neurodegeneration. LANDO activity can be measured by any means known in the art.

In some embodiments, LANDO activity can be determined by measuring neuroinflammation. For example, measuring neuroinflammation can comprise measuring the level of a pro-inflammatory cytokine, an anti-inflammatory cytokine, or a combination of pro-inflammatory cytokines and anti-inflammatory cytokines. In specific embodiments, measuring neuroinflammation comprises measuring the level of TNFα (tumor necrosis factor alpha), IL (interleukin)-10, IL-6, or CCL5 (C—C motif chemokine ligand 5).

In other embodiments, LANDO activity can be determined by measuring β-amyloid clearance or aggregation. For example, β-amyloid plaque number and size can be measured in the hippocampus or cortex using any method known in the art, including but not limited to immunohistochemistry. In other embodiments, secondary β-amyloid uptake is measured using any method known in the art, including but not limited to using labeled β-amyloid that has been labeled with two different labels wherein the first labeled β-amyloid is initially added to medium surrounding cells or tissue and detection of the first label is an indication of primary β-amyloid uptake, and wherein the second labeled β-amyloid is subsequently added to the culture medium and detection of the second label is an indication of secondary β-amyloid uptake. The detection of the first and/or second label can be performed with any method known in the art, including but not limited to flow cytometry.

In other embodiments, LANDO activity can be determined by measuring β-amyloid receptor recycling. In some of these embodiments, the β-amyloid receptor that is measured is at least one of CD36 (cluster of differentiation 36), TLR4 (toll-like receptor 4), and TREM2 (triggering receptor expressed on myeloid cells 2). Any method known in the art can be used to measure β-amyloid receptor recycling, including immunocytochemistry and flow cytometry.

In still other embodiments, LANDO activity can be determined by measuring the association of LC3 to endosomal membranes in response to β-amyloid.

In order to identify molecules or compositions that modulate LANDO activity, but do not have an effect on the LAP pathway, the methods can comprise measuring LAP activity, such as phagocytosis (i.e., phagosome maturation, degradation of engulfed pathogens) mediated by LAPosomes (single membrane phagosome). In other embodiments, the methods and compositions that modulate LANDO activity do not have an effect on autophagy (mediated by double-membrane phagosomes).

Generally, molecules or compositions that modulate LANDO activity can be identified by any screening assay known in the art. For example, a first level of LANDO activity can be measured prior to contact with candidate molecules. A second level of LANDO activity can then be measured following contact with the candidate molecules. Molecules can be selected based on the relative first and second level of LANDO activity, before and after contact with the candidate molecules. Likewise, the level of LANDO activity could be measured in a test cell, tissue, or animal and in a control cell, tissue, or animal following exposure to the candidate molecule. In such an embodiment, the candidate molecule would be selected if the level of LANDO activity is modulated in the test cell, tissue, or animal when compared to the control cell, tissue, or animal. Similarly, the level of LANDO activity could be measured following contacting of the candidate molecule with a LANDO-deficient cell, tissue, or animal. In such an embodiment, the candidate molecule could be selected if LANDO activity was restored in the LANDO-deficient cell, tissue, or animal when compared to a wild type control.

Accordingly, candidate molecules can be selected that modulate (i.e., increase or decrease) the level of LANDO activity. A modulated level of LANDO activity can be an increase of LANDO activity, for instance an increase of at least 1.2, 1.5, 2, 3, 4, 5, 6, 7, 8, 10, 20, 50 times or more relative to an appropriate control. Alternatively, modulation can be a decrease of the level of LANDO activity, for instance a decrease of at least 1.2, 1.5, 2, 3, 4, 5, 6, 7, 8, 10, 20, 50 times or more relative to an appropriate control. In some embodiments, the increase or decrease in LANDO activity is a statistically significant increase or decrease as determined by methods known in the art.

The cells or tissue used for identifying modulation of LANDO activity could be any cell or tissue in which LANDO activity can be measured. In some embodiments the cell or tissue is a bone marrow-derived macrophage or a culture of bone marrow-derived macrophages. In other embodiments, the cell or tissue is a microglial cell or a culture of microglial cells. In still other embodiments, the cell or tissue is a myeloid cell or a culture of myeloid cells. For example, the bone marrow-derived macrophages, microglial cells, or myeloid cells can be from a LANDO-deficient animal (e.g., the genetically modified non-human animals disclosed herein). In specific embodiments, bone marrow-derived macrophages are isolated from Rubicon or ATG deficient mice. In alternative embodiments, bone marrow-derived macrophages are isolated from Atg16L^{ΔWD} mice or mice lacking the WD-domain of Atg16L (also referred to herein as Atg16L WD-domain deficient mice).

Methods for identifying a molecule or composition that modulates LANDO activity can also be tested in vivo in the genetically modified non-human animals comprising a microglial LANDO knockdown or knockout disclosed herein. These methods can comprise measuring a first LANDO activity in the genetically modified non-human animal, then administering a candidate compound to the genetically modified non-human animal and measuring a second LANDO activity after administration to determine the effect on LANDO activity by the candidate compound. These measurements can also be compared to the effects of the candidate compound on a control animal in which the LANDO pathway is intact.

An analysis of the response of cells, tissues, or an animal to the candidate agent may be performed at any time following treatment with the agent. For example, the cells may be analyzed 1, 2, or 3 days, sometimes 4, 5, or 6 days, sometimes 8, 9, or 10 days, sometimes 14 days, sometimes 21 days, sometimes 28 days, sometimes 1 month or more after contact with the candidate agent, e.g., 2 months, 4 months, 6 months or more. In some embodiments, the analysis includes analysis at multiple time points. The selection of the time point(s) for analysis will be based upon the type of analysis to be performed, as will be readily understood by the ordinarily skilled artisan.

Candidate agents of interest for screening include known and unknown compounds that encompass numerous chemical classes, primarily organic molecules, which may include organometallic molecules, inorganic molecules, genetic sequences, vaccines, peptides, polypeptides, antibodies, antigen-binding proteins, agents that have been approved pharmaceutical for use in a human, etc.

Candidate agents include organic molecules including functional groups necessary for structural interactions, particularly hydrogen bonding, and typically include at least an amine, carbonyl, hydroxyl or carboxyl group, frequently at least two of the functional chemical groups. The candidate agents often include cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Molecules and compounds isolated by the methods disclosed herein can be formulated as pharmaceutical compositions for administration according to the methods disclosed herein.

### EMBODIMENTS

1. A method for decreasing neuroinflammation or neurodegeneration in a LC3-associated endocytosis (LANDO)-deficient subject comprising administering an effective amount of a pharmaceutical composition that activates or enhances the LANDO pathway, wherein said administration of an effective amount of a pharmaceutical composition that activates or enhances the LANDO pathway decreases neuroinflammation or neurodegeneration.

2. The method of embodiment 1, wherein said pharmaceutical composition that activates or enhances the LANDO pathway has no significant effect on LC3-associated phagocytosis (LAP).

3. The method of embodiment 1 or 2, wherein said LANDO-deficient subject has reduced expression of at least one of: Beclin1, VPS34, ATG5, ATG7, ATG4, LC3A, LC3B, Rubicon, and Atg16L WD-domain; when compared to a subject not deficient in LANDO.

4. The method of any one of embodiments 1-3, wherein said LANDO-deficient subject has reduced expression of Rubicon, ATG5, or Atg16L WD-domain when compared to a subject not deficient in LANDO.

5. The method of any one of embodiments 1-4, further comprising detecting failed clearance of β-amyloid prior to administering an effective amount of said pharmaceutical composition.

6. The method of any one of embodiments 1-5, wherein said decreased neuroinflammation or neurodegeneration comprises any one of: reduced expression of pro-inflammatory genes, reduced β-amyloid deposition or plaque formation, reduced tau hyperphosphorylation, reduced microglial activation, reduced microglial ramified to ameboid transition, reduced microgliosis, reduced neuronal cell death, reduced electrophysiological impairment, reduced behavior deficits, and reduced memory deficits.

7. A method for treating Alzheimer's disease comprising administering an effective amount of a pharmaceutical composition that activates or enhances the LANDO pathway to a subject diagnosed with Alzheimer's disease or demonstrating symptoms of the disease, wherein said administration of an effective amount of a pharmaceutical composition that activates or enhances the LANDO pathway decreases at least one symptom of Alzheimer's disease.

8. The method of embodiment 7, wherein said subject has reduced expression of at least one of: Beclin1, VPS34, ATG5, ATG7, ATG4, LC3A, LC3B, Rubicon, and Atg16L WD-domain; when compared to a subject not deficient in LANDO.

9. The method of embodiment 7 or 8, wherein said subject has reduced expression of Rubicon, ATG5 or Atg16L WD-domain when compared to a subject not deficient in LANDO.

10. The method of any one of embodiments 7-9, further comprising detecting failed clearance of β-amyloid prior to administering an effective amount of said pharmaceutical composition.

11. A method for clearing β-amyloid in a subject deficient in β-amyloid clearance comprising administering an effective amount of a pharmaceutical composition that activates or enhances the LANDO pathway.

12. The method of embodiment 11, wherein said subject is a LANDO-deficient subject.

13. The method of embodiment 12, wherein said subject has reduced expression of at least one of: Beclin1, VPS34, ATG5, ATG7, ATG4, LC3A, LC3B, Rubicon, and Atg16L WD-domain; when compared to a subject not deficient in LANDO.

14. The method of embodiment 12 or 13, wherein said subject has reduced expression of Rubicon, ATG5 or Atg16L WD-domain when compared to a subject not deficient in LANDO.

15. The method of any one of embodiments 11-14, wherein said subject comprises β-amyloid accumulation in at least one of the cortex and hippocampus prior to administration of said pharmaceutical composition.

16. The method of embodiment 15, wherein said subject exhibits symptoms of said β-amyloid accumulation prior to administration of said pharmaceutical composition.

17. A method for identifying a compound that modulates LANDO activity and does not significantly modulate LAP activity, said method comprising:

measuring a first level of LANDO activity and LAP activity in a cell or tissue;

contacting the cell or tissue with a candidate compound;

measuring a second level of LANDO activity and LAP activity of said cell or tissue after contact with said candidate compound;

comparing said first level of LANDO activity with the second level of LANDO activity and comparing said first level of LAP activity with the second level of LAP activity; and selecting compounds that modulate the LANDO activity and do not significantly modulate the LAP activity.

18. A method for identifying a compound that modulates LANDO activity and does not significantly modulate LAP activity, said method comprising:

contacting a test cell or tissue with a candidate compound;

measuring a first level of LANDO activity and LAP activity of said test cell or tissue after contact with said candidate compound;

measuring a second level of LANDO activity and LAP activity from a control cell or tissue;

comparing said first level of LANDO activity with said second level of LANDO activity and comparing said first level of LAP activity with the second level of LAP activity; and

selecting compounds that modulate the LANDO activity and do not significantly modulate the LAP activity.

19. The method of embodiment 17 or 18, wherein compounds are selected that increase LANDO activity.

20. The method of any one of embodiments 17-19, wherein measuring said first and second level of LANDO activity comprises measuring β-amyloid clearance.

21. The method of any one of embodiments 17-20, wherein measuring said first and second level of LANDO activity comprises measuring recycling of at least one β-amyloid receptor from endosomes to plasma membrane.

22. The method of embodiment 21, wherein said at least one β-amyloid receptor is selected from CD36, TLR4, and TREM2.

23. The method of any one of embodiments 17-22, wherein measuring said first and second level of LAP activity comprises measuring phagocytosis.

24. The method of any one of embodiments 17-23, wherein said cell or tissue comprises a bone marrow-derived macrophage or a culture of bone marrow-derived macrophages, a microglial cell or a culture of microglial cells, or a myeloid cell or a culture of myeloid cells.

25. The method of embodiment 24, wherein said bone marrow-derived macrophage, microglial cell, or myeloid cell is derived from LANDO-deficient mice.

26. The method of embodiment 25, wherein said LANDO-deficient mice are Rubicon deficient, ATG5 deficient or Atg16L WD-domain deficient.

27. The method of any one of embodiments 17-26, wherein said selected molecule modulates LANDO activity when administered to a subject.

28. The method of embodiment 27, wherein said subject is a LANDO-deficient subject.

29. The method of embodiment 28, wherein said LANDO-deficient subject has reduced expression of at least one of. Beclin1, VPS34, ATG5, ATG7, ATG4, LC3, Rubicon, and Atg16L WD-domain; when compared to a subject not deficient in LANDO.

30. The method of embodiment 28 or 29, wherein said LANDO-deficient subject has reduced expression of Rubicon, ATG5 or Atg16L WD-domain when compared to a subject not deficient in LANDO.

31. The method of any one of embodiments 28-30, wherein said LANDO-deficient subject exhibits neuroinflammation or neurodegeneration.

32. A pharmaceutical composition comprising a molecule selected by the method of any one of embodiments 17-31.

33. Use of a pharmaceutical composition that activates or enhances the LANDO pathway for decreasing neuroinflammation or neurodegeneration or treating Alzheimer's disease according to the methods of embodiments 1-6 or 7-10, respectively.

34. Use of a pharmaceutical composition that activates or enhances the LANDO pathway according to the method of any one of embodiments 1-16 or that is identified by the method of any one of embodiments 17-30 as a medicament.

35. A pharmaceutical composition that activates or enhances the LANDO pathway for use in treating a neuroinflammatory disorder, neurodegenerative disorder, or Alzheimer's disease in a LANDO-deficient subject, said use comprising administering an effective amount of a pharmaceutical composition that activates or enhances the LANDO pathway to the subject.

36. The pharmaceutical composition of embodiment 35, wherein said subject has reduced expression of at least one of: Beclin1, VPS34, ATG5, ATG7, ATG4, LC3A, LC3B, Rubicon, and Atg16L WD-domain; when compared to a subject not deficient in LANDO.

37. A mouse model of neuroinflammation or neurodegeneration comprising microglial LANDO knockdown or knockout and at least one additional genetic modification that contributes to neuroinflammation or neurodegeneration.

38. The mouse model of embodiment 37, wherein said microglial LANDO knockdown or knockout targets at least one of Rubicon, ATG5, and Atg16L WD-domain.

39. The mouse model of embodiment 37 or 38, wherein said microglial LANDO knockdown or knockout targets Rubicon.

40. The mouse model of any one of embodiments 37-39, wherein said microglial LANDO knockdown or knockout is tissue-specific.

41. The mouse model of embodiment 40, wherein said microglial LANDO knockdown or knockout is specific to cells of the myeloid lineage and microglia.

42. The mouse model of any one of embodiments 37-41, wherein said microglial LANDO knockdown or knockout is mediated by a site-specific recombinase system.

43. The mouse model of embodiment 42, wherein said site-specific recombinase system comprises Cre/lox.

44. The mouse model of embodiment 42 or 43, wherein expression of a site-specific recombinase is under the control of the lysozyme 2 promoter.

45. The mouse model of any one of embodiments 40-44, wherein said knockdown or knockout targets ATG5 and/or Atg16L WD-domain.

46. The mouse model of any one of embodiments 37-45, wherein said at least one additional genetic modification that contributes to neuroinflammation or neurodegeneration comprises mutations or expression of transgenic molecules that lead to overexpression of a mutated amyloid precursor protein (APP) present in familial Alzheimer's disease (FAD).

47. The mouse model of embodiment 46, wherein said mutated amyloid precursor protein comprises at least one of K670N, M671L, I716V, and V717I in relation to human APP(695).

48. The mouse model of embodiment 47, wherein said mouse model transgenically expresses mutant human APP(695) comprising all of the following mutations: K670N, M671L, I716V, and V717I.

49. The mouse model of embodiment 48, wherein expression of mutant human APP(695) is regulated by a tissue-specific promoter that is expressed in the central nervous system.

50. The mouse model of embodiment 49, wherein expression of mutant human APP(695) is under the regulation of the murine Thy1 promoter.

51. The mouse model of any one of embodiments 46-50, wherein said mouse model transgenically expresses mutant human presinilin 1 comprising a M146L mutation and a L286V mutation.

52. The mouse model of embodiment 51, wherein expression of mutant human presinilin 1 is regulated by a tissue-specific promoter that is expressed in the central nervous system.

53. The mouse model of embodiment 52, wherein expression of mutant human presinilin 1 is under the regulation of the murine Thy1 promoter.

54. The mouse model of any one of embodiments 46-53, wherein said mouse model comprises a 5×FAD transgenic mouse or a Atg16L^{ΔWD} mouse transgenically expressing a mutant human APP(695) with the following mutations: K670N, M671L, I716V, and V717I and transgenically expressing a mutant human presinilin 1 comprising a M146L mutation and a L286V mutation.

55. The mouse model of any one of embodiments 37-54, wherein said microglial LANDO knockdown or knockout increases penetrance of neuroinflammation or neurodegeneration, reduces age of onset of neuroinflammation or neurodegeneration, or both increases penetrance and reduces age of onset of neuroinflammation or neurodegeneration, when compared to a mouse lacking microglial LANDO knockdown or knockout.

56. A method of making a mouse model of neuroinflammation or neurodegeneration comprising microglial LANDO knockdown or knockout and at least one additional genetic modification that contributes to neuroinflammation or neurodegeneration, wherein said method comprises knocking down or knocking out LANDO in microglial tissues in a mouse comprising at least one additional genetic modification that contributes to neuroinflammation or neurodegeneration.

57. The method of embodiment 56, wherein said method further comprises introducing said at least one additional genetic modification that contributes to neuroinflammation or neurodegeneration.

58. The method of embodiment 56, wherein said method comprises crossing a mouse comprising microglial LANDO knockdown or knockout with a mouse comprising at least one additional genetic modification that contributes to neuroinflammation or neurodegeneration.

59. The method of any one of embodiments 56-58, wherein said microglial LANDO knockdown or knockout targets at least one of Rubicon, ATG5, and Atg16L WD-domain.

60. The method of any one of embodiments 56-59, wherein said microglial LANDO knockdown or knockout targets Rubicon.

61. The method of any one of embodiments 56-60, wherein said microglial LANDO knockdown or knockout is tissue-specific.

62. The method of embodiment 61, wherein said microglial LANDO knockdown or knockout is specific to cells of the myeloid lineage and microglia.

63. The method of any one of embodiments 56-62, wherein said microglial LANDO knockdown or knockout is mediated by a site-specific recombinase system and wherein said method further comprises generating said mouse comprising microglial LANDO knockdown or knockout using said site-specific recombinase system.

64. The method of embodiment 63, wherein said site-specific recombinase system comprises Cre/lox.

65. The method of embodiment 63 or 64, wherein expression of a site-specific recombinase is under the control of the lysozyme 2 promoter.

66. The method of any one of embodiments 61-65, wherein said knockdown or knockout targets ATG5 and/or the Atg16L WD-domain.

67. The method of any one of embodiments 56-66, wherein said at least one additional genetic modification that contributes to neuroinflammation or neurodegeneration comprises mutations or expression of transgenic molecules that lead to overexpression of a mutated amyloid precursor protein (APP) present in familial Alzheimer's disease (FAD).

68. The method of embodiment 67, wherein said mutated amyloid precursor protein comprises at least one of K670N, M671L, I716V, and V717I in relation to human APP(695).

69. The method of embodiment 68, wherein said mouse model transgenically expresses mutant human APP(695) comprising all of the following mutations: K670N, M671L, I716V, and V717I.

70. The method of embodiment 69, wherein expression of mutant human APP(695) is regulated by a tissue-specific promoter that is expressed in the central nervous system.

71. The method of embodiment 70, wherein expression of mutant human APP(695) is under the regulation of the murine Thy1 promoter.

72. The method of any one of embodiments 67-71, wherein said mouse model transgenically expresses mutant human presinilin 1 comprising a M146L mutation and a L286V mutation.

73. The method of embodiment 72, wherein expression of mutant human presinilin 1 is regulated by a tissue-specific promoter that is expressed in the central nervous system.

74. The method of embodiment 73, wherein expression of mutant human presinilin 1 is under the regulation of the murine Thy1 promoter.

75. The method of any one of embodiments 67-74, wherein said mouse model comprises a 5×FAD transgenic mouse transgenically expressing a mutant human APP(695) with the following mutations: K670N, M671L, I716V, and V717I and transgenically expressing a mutant human presinilin 1 comprising a M146L mutation and a L286V mutation.

76. The method of any one of embodiments 56-75, wherein said microglial LANDO knockdown or knockout increases penetrance or neuroinflammation or neurodegeneration, reduces age of onset of neuroinflammation or neurodegeneration, or both increases penetrance and reduces age of onset of neuroinflammation or neurodegeneration, when compared to a mouse lacking microglial LANDO knockdown or knockout.

77. A mouse model of neuroinflammation or neurodegeneration produced by the method of any one of embodiments 56-76.

78. A method for identifying a compound that modulates neuroinflammation or neurodegeneration, said method comprising:

a) administering a candidate compound to said mouse model of any one of embodiments 37-55 or 77;

b) measuring the effect of said candidate compound on neuroinflammation or neurodegeneration as compared to said mouse model prior to administration of said candidate compound or said mouse model not having been administered said candidate compound; and

c) selecting compounds that modulate neuroinflammation or neurodegeneration.

79. The method of embodiment 78, wherein measuring the effect of said candidate compound on neuroinflammation or neurodegeneration comprises measuring any one of: expression of pro-inflammatory genes, β-amyloid deposition or plaque formation, tau hyperphosphorylation, microglial activation, microglial ramified to ameboid transition, microgliosis, neuronal cell death, electrophysiological impairment, behavior deficits, and memory deficits.

80. The method of embodiment 79, wherein expression of any one of the following pro-inflammatory genes are measured: IL-1β, IL-6, CCL5, and TNFα.

81. The method of embodiment 79, wherein said microglial activation is measured by measuring expression of Iba1.

82. The method of embodiment 79, wherein behavior deficits are measured using a sucrose preference test.

83. The method of embodiment 79, wherein memory deficits are measured using a novel object recognition test, a Y-maze test, or both.

84. Use of a pharmaceutical composition that activates or enhances the LANDO pathway in the manufacture of a medicament for decreasing neuroinflammation or neurodegeneration or treating Alzheimer's disease according to the methods of embodiments 1-6 or 7-10, respectively.

### EXPERIMENTAL

### Example 1. ATG5 and Rubicon-Deficiency Exacerbate β-Amyloid Deposition and Pathology

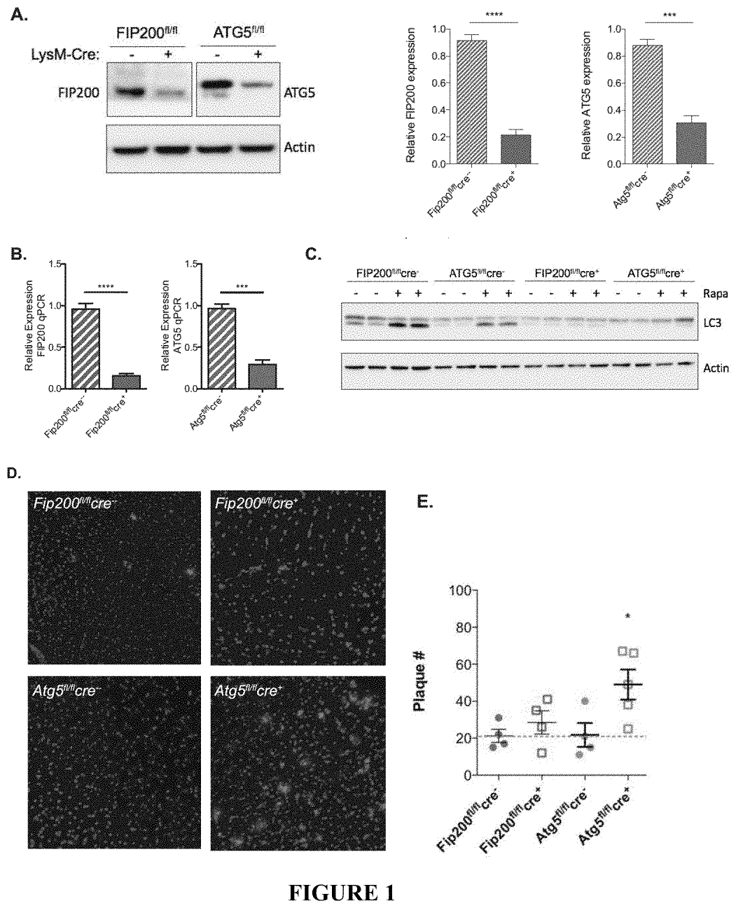

To ascertain the involvement of myeloid autophagy in the establishment and progression of amyloid deposition and neuroinflammation, a murine model was employed in which animals express transgenes of APP and Presenilin1 containing several mutations associated with human familial AD, the 5×FAD (B6.Cg-Tg (APPSwFILon, PSEN1*M146L*L286V) 6799Vas) model (Oakley et al. (2006) J Neurosci 26:10129-10140). These animals accumulate β-amyloid, have increased tau phosphorylation, and eventually show signs of neuronal loss and behavioral changes consistent with neurodegeneration (Oakley et al. (2006)). The 5×FAD transgene was crossed into mice with conditional ablation of the key autophagy regulators FIP200 and ATG5 using lysozyme M (LysM/Lyz2)-Cre-lox recombination, which targets cells of the myeloid lineage and microglia, with an efficiency ranging from 40-90% in microglia (Abram et al. (2014) J Immunol Methods 408:89-100; Ferro et al. (2018) PLoS One 13:e0200013; Pulido-Salgado et al. (2017) J Neuroinflammation 14:54). Primary microglia isolated from LysM-Cre⁺ FIP200^{fl/fl} and ATG5^{fl/fl} mice (referred to as FIP200,cre⁺ and ATG5,cre⁺) showed a significant reduction in mRNA and protein expression when compared to LysM-Cre-littermates (FIG. 1A,B). Moreover, primary microglia from these genotypes displayed a significant reduction in autophagic capacity as measured by decreased LC3-lipidation upon stimulation with rapamycin when compared to cells isolated from Cre⁻ littermates (FIG. 1C). In addition to conditional ablation of FIP200 and ATG5, mice that have germline-deficiency of Rubicon (Rubicon) were included, which has been shown to be an inhibitor of canonical autophagy (Matsunaga et al. (2009) Nat Cell Biol 11:385-396), but also a key component of a non-canonical function of autophagy proteins that modulates inflammatory immune activation (Cunha et al. (2018) Cell 175:429-441, e416; Martinez et al. (2016) Nature 533:115-119).

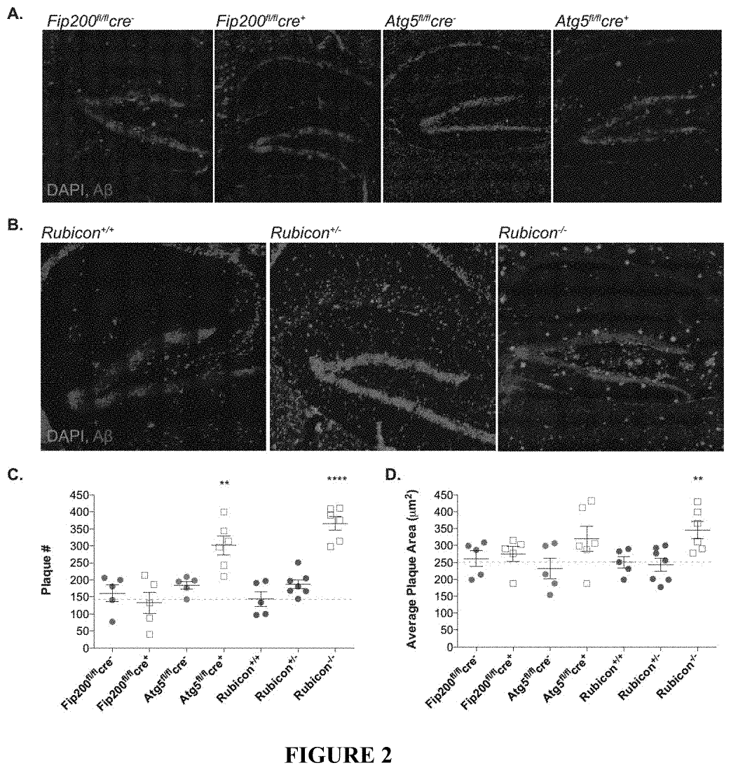

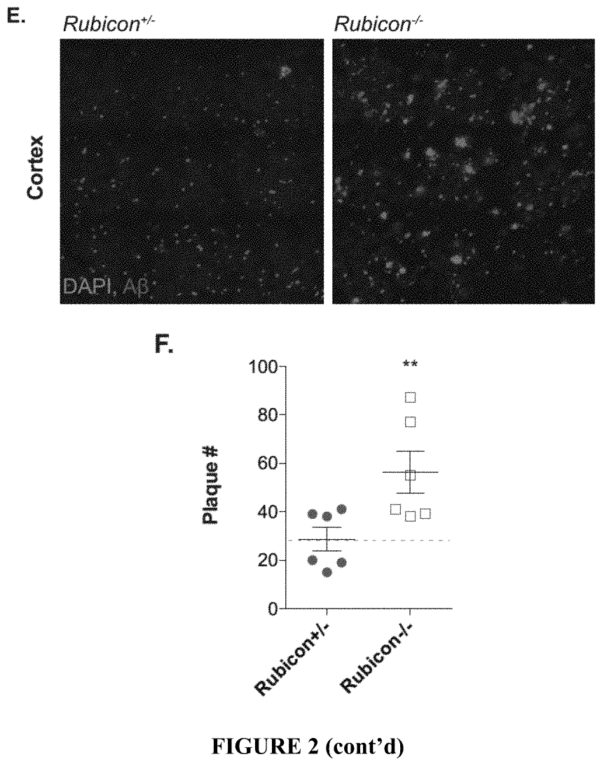

Mice that are autophagy-deficient (5×FAD, FIP200,cre⁺) have no difference in β-amyloid deposition when compared to LysM-Cre⁻ littermates (FIG. 2A,C,D). Interestingly, deletion of ATG5 in the myeloid compartment (5×FAD, ATG5, cre⁺) leads to a significant increase in β-amyloid plaque number and plaque size within the hippocampus (FIG. 2A,C,D) of 5×FAD mice as early as 2.5 months of age. The inconsistency regarding β-amyloid deposition between these two models of autophagy-deficiency suggested an alternative pathway responsible for regulating β-amyloid deposition, separate from canonical autophagy. Consistent with this notion and similar to mice deficient in myeloid ATG5, 5×FAD Rubicon^{-/-} mice displayed an even greater increase in β-amyloid plaque number and plaque size compared to 5×FAD^{tg} Rubicon^{+/-} littermates (FIG. 2B-D). Likewise, both 5×FAD ATG5 cre⁺ and 5×FAD Rubicon^{-/-} mice showed early accumulation of β-amyloid within the cortex, whereas mice deficient for myeloid FIP200 were unaffected compared to LysM-cre⁻ littermates (FIG. 1D,E and FIG. 2E,F). The exacerbation of β-amyloid accumulation within the hippocampus and cortex of the ATG5 and Rubicon-deficient mice but not in mice deficient in FIP200 is suggestive of an alternative function of canonical autophagy proteins in the regulation of amyloid deposition.

### Example 2. LC3 is Recruited to Endosomes Containing β-Amyloid

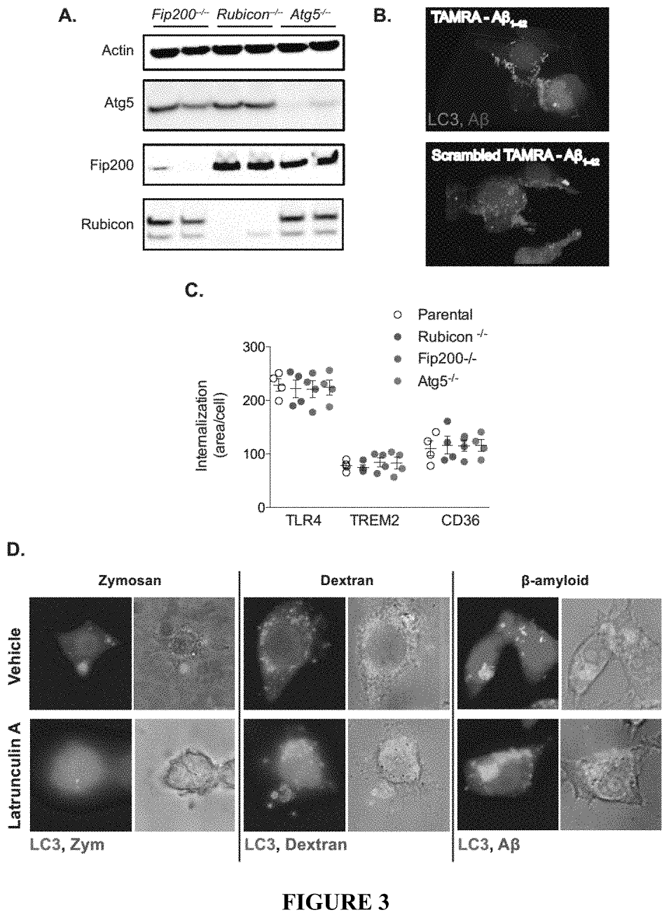

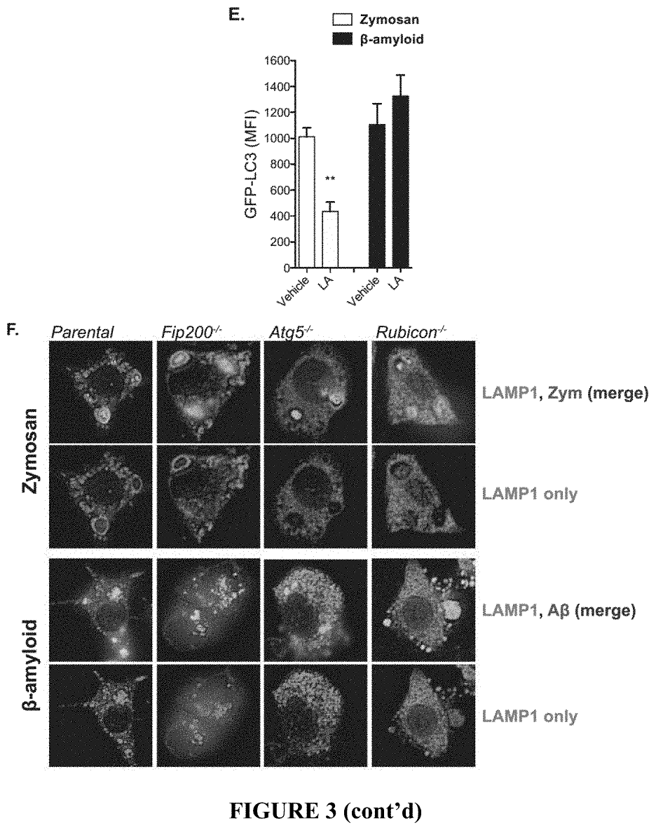

In an attempt to delineate the discrepancies in β-amyloid accumulation in the animal models presented above and the role of the autophagy proteins in this process, BV2 murine microglial cells expressing GFP-LC3 that are deficient in FIP200, ATG5, or Rubicon were engineered using CRISPR/Cas9 (FIG. 3A). Cells were cultured in the presence of neurotoxic oligomeric AP 1-42 labeled with TAMRA. In parental cells, Aβ1-42 induced rapid co-localization of LC3 to the β-amyloid, whereas scrambled Aβ1-42 failed to promote this recruitment (FIG. 3B). Parental cells and those lacking FIP200 displayed recruitment of LC3 to β-amyloid containing endosomes (FIG. 4A). In stark contrast, ATG5 and Rubicon-deficient cells had a robust reduction in membrane-associated LC3 (FIG. 4A,B). Inhibition of phagocytosis using latrunculin A (de Oliveira and Mantovani (1988) Life Sci 43:1825-1830; Oliveira et al. (1996) Chem Biol Interact 100:141-153), prevented the internalization of the phagocytic substrate zymosan but had no effect on either the endocytic substrate dextran or β-amyloid (FIG. 3D, FIG. 4C), confirming that uptake of β-amyloid in this model occurs primarily through receptor-mediated endocytosis. Additionally, inhibition of phagocytosis did not alter the association of LC3 to endosomal membranes in response to β-amyloid (FIG. 3E). Taken together, these data suggest that both ATG5 and Rubicon are necessary for recruitment of LC3 upon Aβ-induced endocytosis, whereas FIP200 is dispensable.

These findings with ATG5, Rubicon, and FIP200 are consistent with a well-established non-canonical function of autophagy proteins in the LC3-associated phagocytosis (LAP) pathway (Heckmann et al. (2017) J Mol Biol 429:3561-3576). Deficiencies in LAP have been shown to reduce the ability of a cell to degrade phagosome cargo, including dying cells, fungi, and bacteria by impairing phagosome maturation and lysosomal interaction (Heckmann et al. (2017); Martinez et al. (2011) Proc Natl Acad Sci USA 108:17396-17401; Martinez et al. (2016) Nature 533:115-119; Martinez et al. (2015) Nat Cell Biol 17:893-906). This may in part explain the increased β-amyloid accumulation observed in these deficient murine models. To test this idea, a pulse-chase assay was performed using oligomeric TAMRA-labeled Aβ1-42. Ablation of either ATG5 or Rubicon, however, had no effect on either the endocytosis or degradation of β-amyloid (FIG. 4D). Indeed, earlier studies had shown no role for LAP in the maturation of dextran-containing endosomes or in the degradation of internalized EGFR following ligation (Cunha et al. (2018) Cell 175:429-441, e416). Consistent with these reports, in the absence of Rubicon and ATG5, phagosomes containing zymosan have a reduced maturation and association with lysosomes as measured by co-localization with the lysosome marker LAMP1, while this association with β-amyloid-containing endosomes was unaffected (FIG. 3F, 4E). Together, these results argue against a role for LAP in regulating β-amyloid endocytosis and degradation. Based on these observations, the term LC3-Associated eNDOcytosis (LANDO) is proposed to describe this effect, and these data suggest that LANDO is distinct from LAP and may have a unique role in β-amyloid clearance in vivo without affecting degradation of engulfed amyloid.

A previous study has shown that the recycling of the putative β-amyloid receptors TREM2 and CD36 to the plasma membrane, following receptor-mediated endocytosis, required two autophagy proteins, Beclin1 and VPS34 (Lucin et al. (2013) Neuron 79:873-886). Therefore, the role of FIP200, ATG5, and Rubicon in recycling of TREM2, CD36, and TLR4 was examined (the latter has been suggested to be another putative β-amyloid receptor (Reed-Geaghan et al. (2009) J Neurosci 29:11982-11992; Song et al. (2011) J Neuroinflammation 8:92). First, it was assessed if subsequent rounds of β-amyloid endocytosis would be impacted in the microglial model. BV2 cells were treated with Aβ1-42 labeled with a 488-fluor and measured primary β-amyloid uptake. Consistent with the clearance assay, the loss of FIP200, ATG5, or Rubicon had no effect on primary uptake of β-amyloid (FIG. 4F). TAMRA-labeled Aβ1-42 was next added and allowed for a second round of receptor-mediated endocytosis to occur. Again, the amount of internalized TAMRA-Aβ1-42 was quantified and a reduction in secondary uptake was observed in ATG5 and Rubicon-deficient cells but not those deficient in FIP200 (FIG. 4F). These results are supportive of a failure to return receptors to the plasma membrane following initial internalization, since secondary rounds of uptake were decreased but primary uptake was unaffected.

To better understand the abrogation of secondary uptake, the impact of loss of FIP200, ATG5, and Rubicon on receptor recycling was evaluated, using an established method (Lucin et al. (2013) Neuron 79:873-886). In BV2 microglia, depletion of FIP200 had no effect on the internalization (FIG. 3C) or recycling of CD36, TLR4, or TREM2 (FIG. 4G,H). Strikingly, both ATG5 and Rubicon depletion led to abrogation of receptor recycling for all three receptors (FIG. 4G,H) while initial internalization was unaffected (FIG. 3C). Furthermore, since TREM2 is the most well-characterized β-amyloid receptor that activates β-amyloid endocytosis (Doens and Fernandez (2014) J Neuroinflammation 11:48; Ries and Sastre (2016) Front Aging Neurosci 8:160; Ulland et al. (2017) Cell 170:649-663, e613; Wang et al. (2015) Cell 160:1061-1071; Zhao et al. (2018) Neuron 97:1023-1031, e1027), the ability to recycle TREM2 in primary microglia cells isolated from Rubicon^{-/-} mice was evaluated. Rubicon-deficiency dramatically reduced recycling of TREM2 (FIG. 4I,J). These findings demonstrate a role for ATG5 and Rubicon in the regulation of receptor recycling upon internalization of β-amyloid. Moreover, when taken together, the abrogation of receptor recycling but not internalization can explain the lack of alteration of primary uptake but an impact on secondary rounds of endocytosis that are ATG5 and Rubicon dependent, but FIP200 independent. Therefore, LANDO is required for the recycling of internalized β-amyloid receptors to the plasma membrane.

To further explore this phenomenon, the RAW264.7 myeloid cell line was employed, and it was found that TLR4, TREM2, and CD36 effectively recycled to the plasma membrane following antibody-induced internalization. This recycling was dramatically impaired upon CRISPR/Cas9-mediated ablation of Rubicon or ATG5 (FIG. 5A), upon expression of a dominant negative ATG4 (FIG. 5A), or upon expression of the LC3-specific protease, RavZ (FIG. 5A, FIG. 6B). The requirement for ATG4, which converts LC3 to LC3-I (Kabeya et al. (2004) J Cell Sci 117:2805-2812) and the effect of RavZ strongly suggest that lipidation of LC3-family proteins is required for recycling of these receptors.

The analysis was then extended, using primary, bone marrow-derived macrophages (BMDMs) from animals with myeloid ablation of a number of genes required for autophagy and/or LAP (FIG. 5C, FIG. 6B). Ablation of Beclin1, VPS34, ATG5, or ATG7, required for both autophagy and LAP (Martinez et al. (2015) Nat Cell Biol 17:893-906), prevented recycling of TLR4, TREM2, and CD36, as did Rubicon, required for LAP but not autophagy (Martinez et al. (2015)). In contrast, no effects were seen upon ablation of ULK1, FIP200, or ATG14, all required for autophagy, but dispensable for LAP (Martinez et al. (2015)). Therefore, the requirements for recycling of these receptors to the plasma membrane are identical to those for LAP and distinct from those of autophagy. However, since no effects on the degradation of Aβ were observed (FIG. 4D), and since antibody-induced receptor internalization is via endocytosis and not phagocytosis, it is concluded that it is the association of LC3 with endosomes (LANDO) that displays these genetic requirements.

### Example 3. LANDO Protects Against β-Amyloid Induced Neuroinflammation

These data suggest that the exacerbation of β-amyloid accumulation in the LANDO-deficient mice is a result of impaired recycling of putative β-amyloid receptors leading to extracellular deposition. β-amyloid, especially the Aβ1-42 oligomer and fibril, is an established instigator of neuroinflammation (Cai et al. (2014) Int J Neurosci 124:307-321). Since a vital role for LAP in regulating inflammatory immune responses has previously been shown (Martinez et al. (2016) Nature 533:115-119), it was hypothesized that defective LANDO may have a similar effect. Therefore, the production of inflammatory cytokines in the BMDMs exposed to Aβ1-42 oligomers in the above experiment was assessed. Strikingly, those genotypes that displayed defective receptor recycling showed a dramatically elevated TNFα, IL-1β, and IL-6 response to Aβ1-42 (FIG. 6C).

Similarly, the effects of Aβ1-42 on microglia were examined. As expected, BV2 cells treated with Aβ1-42 had elevated pro-inflammatory gene expression, including IL-1β, IL-6, CCL5, and TNFα as reported (Pan et al. (2011) Mol Neurodegener 6:45) and consistent with human disease. FIP200-deficiency failed to have any impact on cytokine expression in response to Aβ1-42 (FIG. 6D). However, loss of LANDO in both ATG5 and Rubicon-deficient cells resulted in a robust increase in all four pro-inflammatory genes evaluated (FIG. 6D).

These findings were further substantiated in primary microglia from Rubicon-deficient mice. A potent hyperactivation of TNFα, IL1-β and IL-6 was observed at both the mRNA (FIG. 6E) and protein levels (FIG. 6F) upon Aβ1-42 exposure in Rubicon^{-/-} microglia. Together, these results suggest a role for LANDO in the modulation and likely the mitigation of inflammatory activation in response to β-amyloid.

Since a strong exacerbation of pro-inflammatory gene expression was observed in response to Aβ1-42 in vitro, it was next assessed if reactive microglial activation and neuroinflammation was present in the murine models. Using Iba1 as a marker for microglial activation (Hoogland et al. (2015) J Neuroinflammation 12:114), hippocampal and cortical sections were assessed to evaluate microglial activation. Consistent with the in vitro findings, myeloid FIP200-deficiency had no effect on the extent of microglial activation in either the hippocampus or cortex of 5×FAD mice (FIG. 7A-C). In contrast, microglial hyperactivation was present in 5×FAD mice deficient in either Rubicon or myeloid ATG5 (FIG. 7A,B). This activation was not constrained to the hippocampus and was also present in the cerebral cortex (FIG. 7A,C).

Hyperactivation of microglia typically leads to the morphological transition of cells from the ramified to the ameboid state, resulting in a decreased phagocytic/endocytic capacity and increased inflammatory polarization (Kim and Joh (2006) Exp Mol Med 38:333-347). Morphological analysis of microglia in the hippocampus of 5×FAD LANDO-deficient mice revealed ramified to ameboid transition, with Rubicon-deficient and myeloid ATG5-deficient mice having a reduction in ramified microglia in the hippocampus when normalized to total microglial population compared to control littermates (FIG. 7D,E). FIP200-deficiency had no effect on microglial state transition when compared to control littermates. Furthermore, in 5×FAD Rubicon-deficient mice, analysis of microglia/plaque-association revealed an increase in plaque-associated microglia (FIG. 7F), suggestive of progressive gliosis in response to β-amyloid.

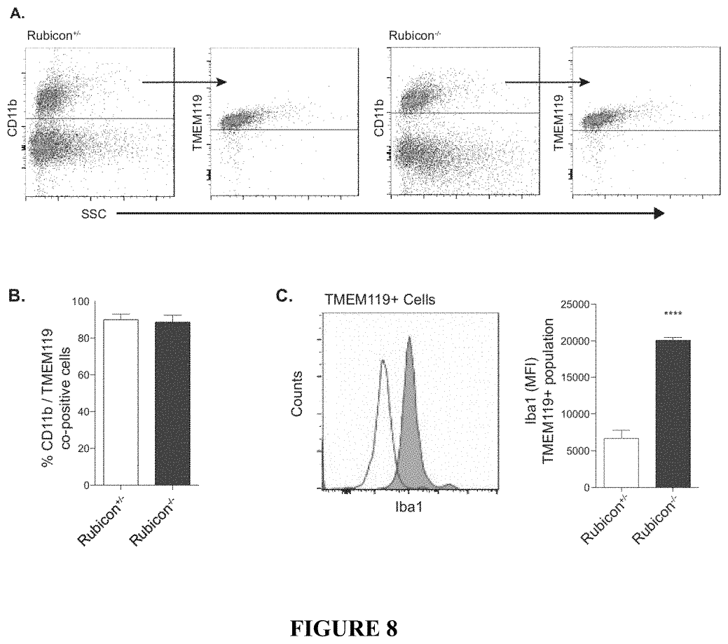

Due to the high level of microglia activation in the ATG5 and Rubicon-deficient mice, and the transition to the more active/reactive ameboid morphology, the most clinically relevant pro-inflammatory cytokines implicated in neuroinflammation in AD were profiled (Shaftel et al. (2008) J Neuroinflammation 5:7). Consistent with what was observed with cultured cells (FIG. 6), Rubicon-deficiency led to significant upregulation of pro-inflammatory cytokines at the transcriptional level in the brain. Using Iba1 expression as a positive control, significant increases in TNFα, IL-1β, IL-6, and a marginal increase in CCL5 within the hippocampus of 5×FAD Rubicon^{-/-} compared to 5×FAD Rubicon^{+/-} littermates were observed (FIG. 7G). To confirm the specificity of using increased Iba1 expression as a control for microglia and not total monocytes, the percentage of brain-infiltrating peripheral monocytes was analyzed. Over 90% of CD11b positive cells in brains from both the 5×FAD Rubicon+/− and ^{-/-} mice were observed to be TMEM119+(a microglia-specific marker (Bennett et al. (2016) Proc Natl Acad Sci USA 113:E1738-1746) (FIG. 8A,B). Furthermore, the TMEM119+ cells isolated from 5×FAD Rubicon^{-/-} mice had higher Iba1 expression compared to Rubicon^{+/-} animals (FIG. 8C).

These results suggest a critical role for Rubicon and myeloid ATG5 in mitigating reactive/inflammatory microglial activation in the 5×FAD model, thereby dampening neuroinflammation. Taken as a whole, these data suggest that LANDO may contribute to not only the regulation of aberrant β-amyloid deposition but also the immune activation of microglial cells in response to β-amyloid exposure.

### Example 4. LANDO-Deficient 5×FAD Mice have Robust Tau Pathology

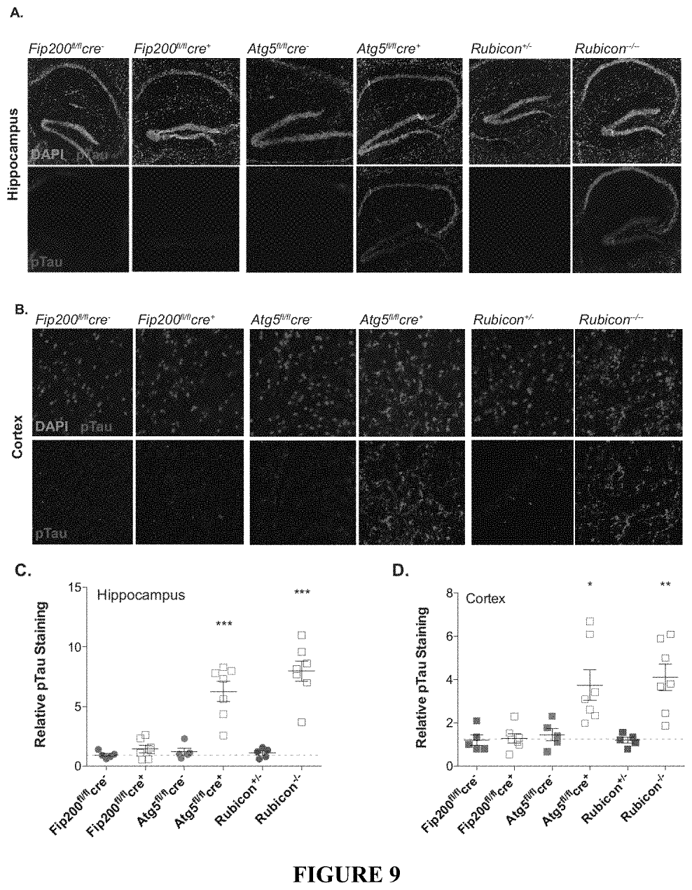

In agreement with human AD, LANDO-deficient 5×FAD animals displayed severe β-amyloid accumulation that promoted reactive microgliosis and neuroinflammation. A marker of progressive AD in both humans and mice is the hyperphosphorylation of the microtubule-stabilizing protein tau. The incidence of tau hyperphosphorylation increases as disease progresses and leads to microtubule de-stabilization and ultimately failure of the microtubule architecture, especially within neuronal axons (Frost et al. (2015) Trends Cell Biol 25:46-53; Gong and Iqbal (2008) Curr Med Chem 15:2321-2328; Noble et al. (2013) Front Neurol 4:83). The phosphorylation state of tau was evaluated and the presence of phospho-tau was identified which paralleled both our β-amyloid and microglial phenotypes. Again, myeloid FIP200 depletion had no impact on the phosphorylation of tau, however loss of either Rubicon or myeloid ATG5 promoted tau hyperphosphorylation throughout the hippocampus (FIG. 9A,C) and the cerebral cortex (FIG. 9B,D). These data suggest that loss of LANDO promotes rapid alterations to tau that are indicative of highly progressive disease.

### Example 5. LANDO-Deficient 5×FAD Mice Display Accelerated Neuronal Death and Impaired Neuronal Function

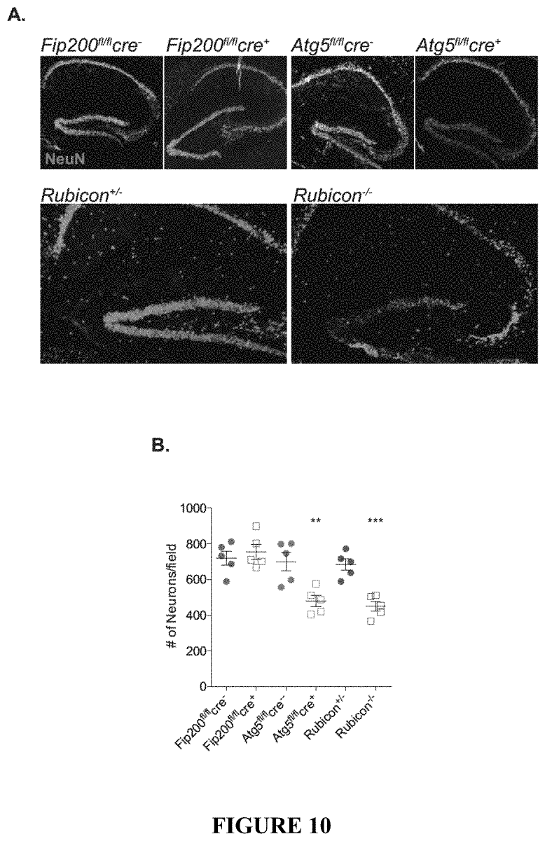

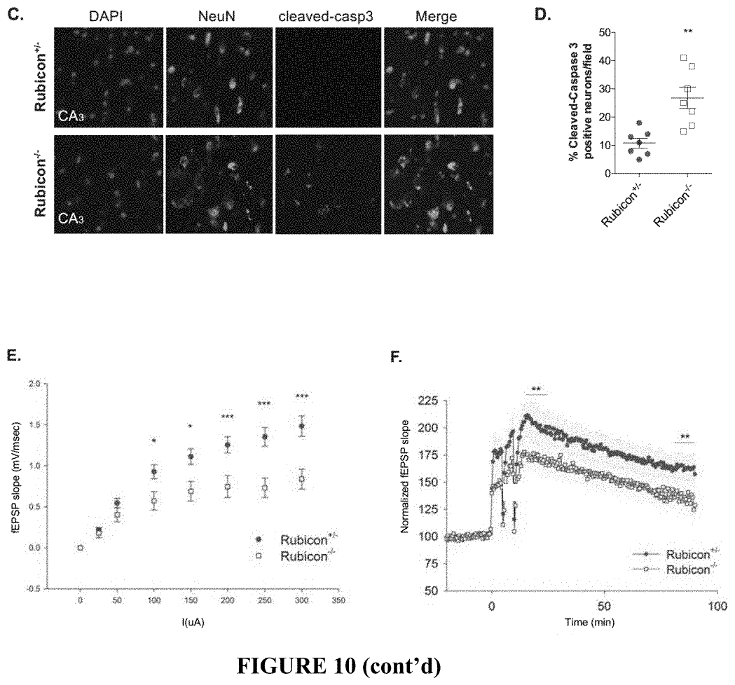

Because tau hyperphosphorylation is likely to promote axonal degeneration and eventual neuronal death, we analyzed cell death within the brains of our 5×FAD models. To evaluate the total number of neurons within the hippocampus, sections were stained with the neuronal nuclei marker, NeuN. 5×FAD Rubicon^{-/-} and myeloid ATG5-deficient mice both have a decrease in NeuN positive neurons in the hippocampus (FIG. 10A,B). FIP200-deficiency had no impact on neuronal number within the hippocampus.

Since a reduction in the number of neurons was observed in both of the LANDO-deficient models, but not the autophagy-deficient model, cell death was evaluated in brains isolated from the more penetrant genotype (5×FAD Rubicon^{-/-}). To measure neuronal apoptosis specifically, immunofluorescence microscopy staining was performed for cleaved-caspase 3. There was a robust increase in cleaved-caspase 3 positive neurons in 5×FAD Rubicon-deficient mice compared to littermate controls in the CA3-field of the hippocampus (FIG. 10C,D). This region including the CA1-field has been implicated as one of the first sites of neuronal dysfunction and neuronal death in AD (Belvindrah et al. (2014) Front Cell Neurosci 8:63; Padurariu et al. (2012) Psychiatr Danub 24:152-158; Zhang et al. (2013) Brain 136:1432-1445). In combination, these results support the idea that control of β-amyloid deposition and microglia/neuroinflammation by LANDO is critical for preventing hyperphosphorylation of tau, neuronal apoptosis, and degeneration.

Electrophysiological assessment of neuronal function was next performed in an effort to substantiate and define the results demonstrating neuron loss in the CA3-field. Through the use of hippocampal electrophysiology, it was found that 5×FAD Rubicon^{-/-} mice had a large reduction in synaptic transmission and as a consequence impaired long-term potentiation (LTP) when compared to littermate controls (FIG. 10E,F). The neuronal death and major impairment in neuronal physiology was surprising, as 5×FAD mice do not begin to show signs of cell death and functional impairment until 5-6 months of age, and to a lesser extent (Eimer and Vassar (2013) Mol Neurodegener 8:2). Therefore, when LANDO is defective, neuronal cell death induced by β-amyloid is accelerated, particularly within the pre-synaptic neurons of the hippocampus. Reduction of this neuronal population is confirmed by inhibition of pre-synaptic transmission and LTP.

### Example 6. LANDO-Deficiency Accelerates Behavioral and Memory Impairment in 5×FAD Mice

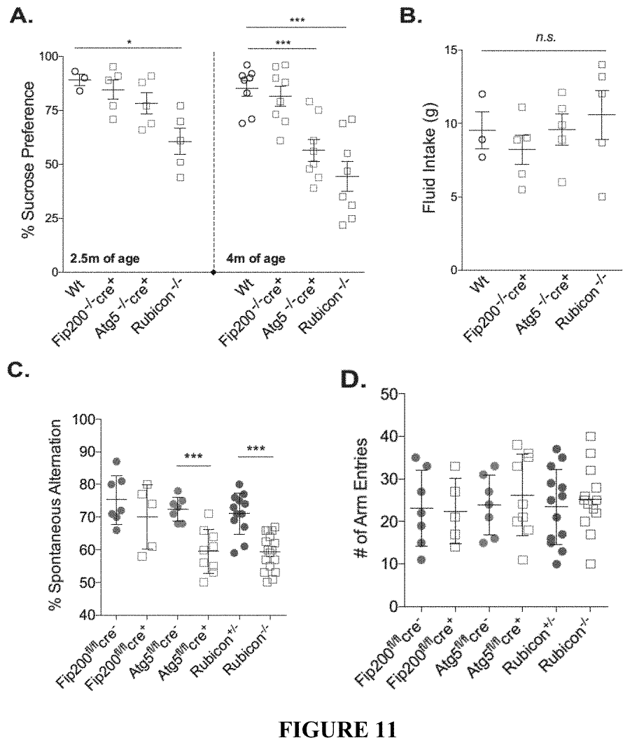

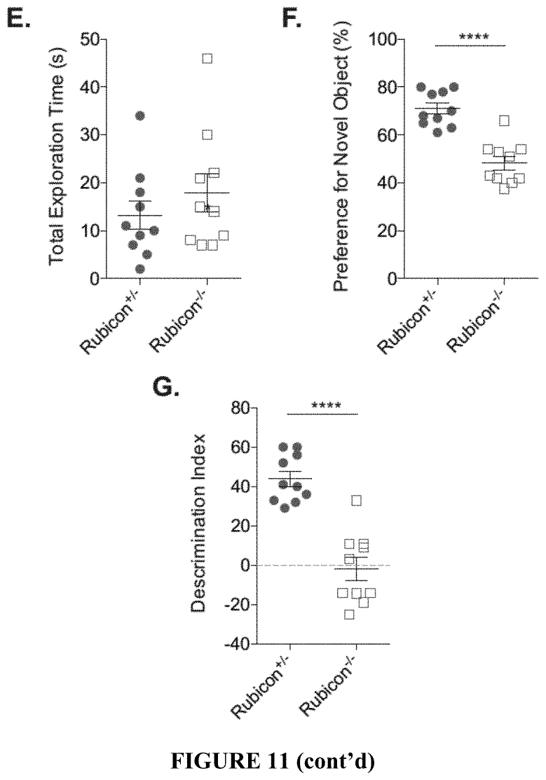

Thus far, these data are supportive of a physiological role for LANDO in the mitigation and protection against neuroinflammation and immune-mediated aggregate (β-amyloid) removal. Mice were therefore subjected to a variety of well-characterized behavioral tests known to be affected at late stages in the 5×FAD model. In advanced AD, patients often complain of anhedonia, or the inability to sense pleasure (Naudin et al. (2015) Psychiatry Res 228:228-232; Reichman and Coyne (1995) J Geriatr Psychiatry Neurol 8:96-99), which can be analyzed in mice using a sucrose preference test (SPT) (Briones et al. (2012) Br J Pharmacol 165:897-907; Liu et al. (2018) Nat Protoc 13:1686-1698). 5×FAD mice that were deficient in myeloid FIP200 showed no variation in their preference for sucrose water when compared to wild-type 5×FAD animals, suggesting they have intact reward behavior (FIG. 11A). In contrast, both Rubicon and myeloid ATG5-deficient mice presented with anhedonia. Both genotypes were at approximately 50% sucrose preference, or simple chance (FIG. 11A), by 4 months of age. Interestingly, behavioral and memory deficits do not typically begin to show significant differences in 5×FAD animals until at least 5-7 months of age (Girard et al. (2014) Hippocampus 24:762-772; Ohno (2009) Neurobiol Learn Mem 92:455-459). 5×FAD mice that were LANDO-deficient presented with anhedonia as young as 2.5 months old. No variations in total fluid intake between genotypes was observed (FIG. 11B).

Results from the SPTs suggested a more pervasive memory impairment. Therefore, two routinely used tests for short-term and working short-term memory were employed, the novel object recognition test (NOR) and the Y-maze test respectively. Consistent with their performance in the SPT, 5×FAD Rubicon^{-/-} and myeloid ATG5-deficient mice had a reduction in spontaneous alternation (FIG. 11C) without having a decrease in total arm entries (FIG. 11D) in the Y-maze test. Moreover, short-to-medium term memory was drastically reduced in the 5×FAD Rubicon-deficient mice, as measured by NOR. Rubicon-deficiency resulted in a decrease in novel object preference, and an almost complete reduction in their discrimination index (FIG. 11F,G). These analyses illustrate the importance for the molecular regulation of immune function by LANDO in maintaining CNS integrity and immune function upon amyloid deposition, allowing for homeostasis in memory and behavior.

### Example 7. Deletion of Atg16L WD-Domain Results in Spontaneous AD-Like Pathology

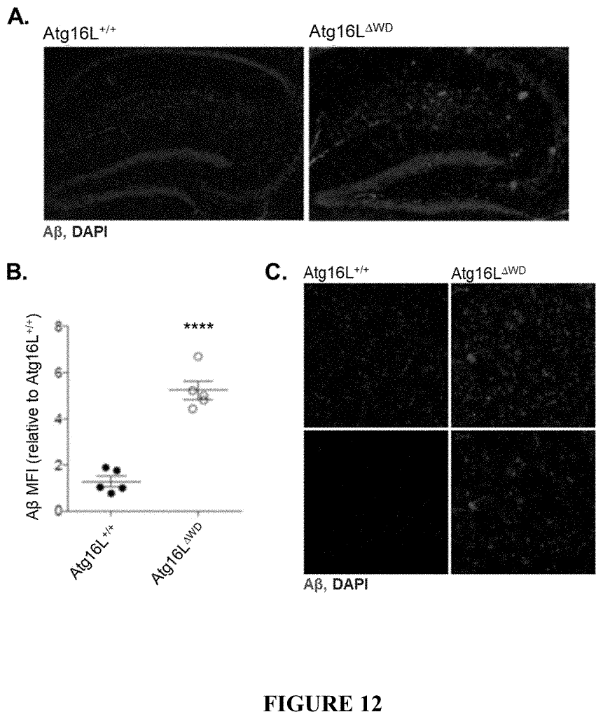

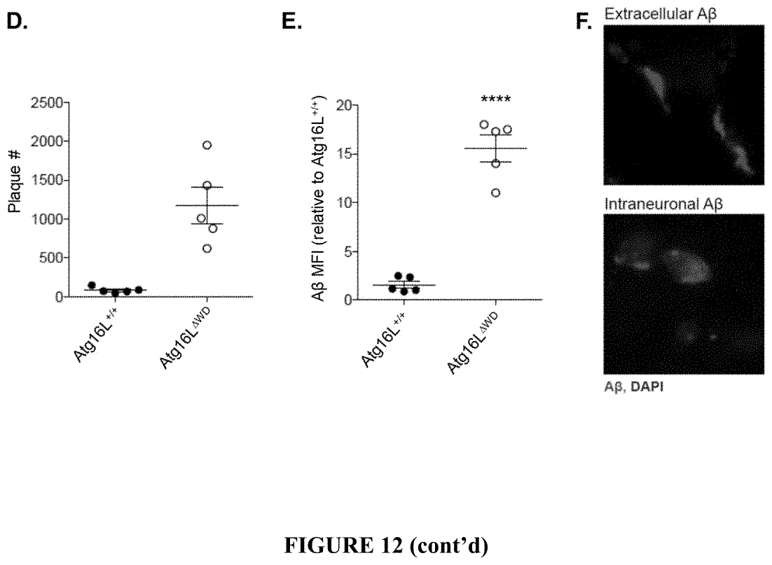

To determine the importance of the WD-domain of Atg16L in central nervous system physiology, aged mice lacking the WD-domain of Atg16L (Atg16L^{ΔWD} mice) (Rai et al. 2018 Autophagy 15(4): 599-612) were evaluated. The findings are described in FIGS. 12A-12F, 13A-13F, and 14A-14C.

When compared to littermate controls (Atg16L^{+/-} mice), WD-domain deficient (Atg16L^{ΔWD}) mice aged to two years showed robust deposition of endogenous murine Aβ in both the hippocampus (FIGS. 12A-12B) and throughout the cerebral cortex (FIGS. 12C-12E). Upon closer inspection, Aβ pathology in the WD-domain deficient (Atg16L^{ΔWD}) mice was found to be characterized by a combination of both extracellular aggregates and intraneuronal deposits (FIG. 12F). Although these findings are consistent with what is typically observed in human AD patients, aged mice lacking the WD-domain of Atg16L did not present with dense-cored Aβ plaques that are characteristic of both human disease and those found in mouse models overexpressing mutant forms of human amyloid precursor protein (APP). It is plausible that the lack of plaque formation is a result of inherent biochemical differences between endogenous mouse and human APP as well as the associated Aβ cleavage products. In particular, mouse Aβ₁₋₄₂ is known to have a reduced propensity for forming β-sheet structures compared to human Aβ₁₋₄₂ (PMID: 23700581), which would explain the absence of Aβ plaques in the WD-domain deficient (Atg16L^{ΔWD}) mice where endogenous mouse Aβ is accumulating.

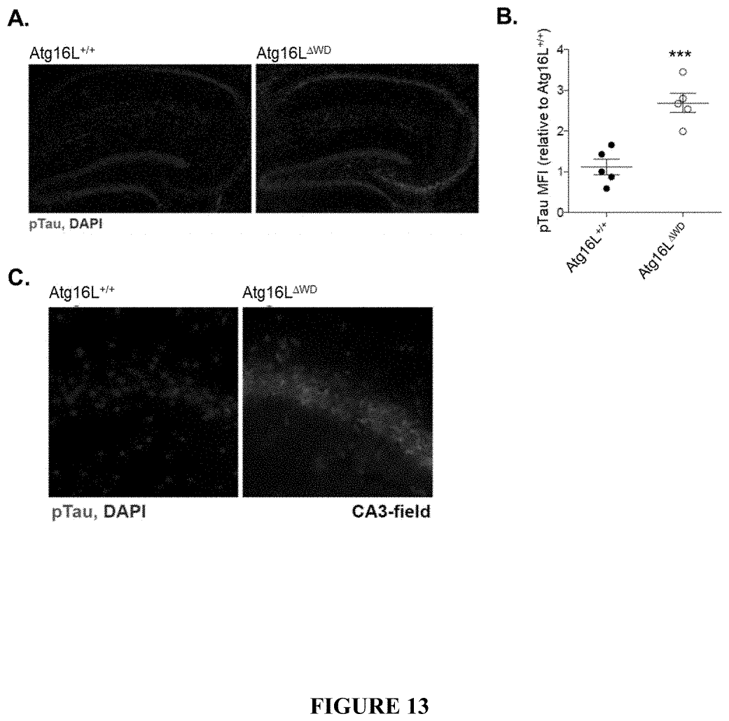

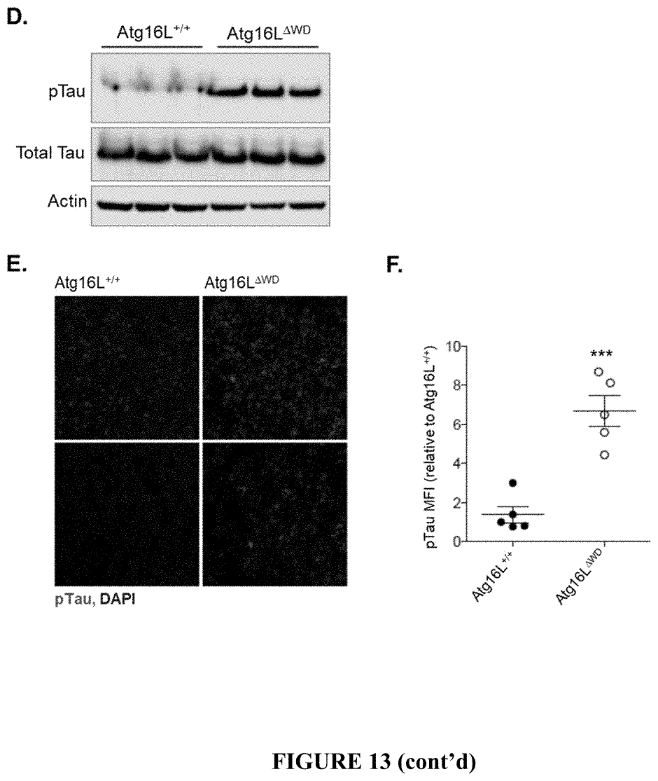

In addition to Aβ deposition, pervasive hyper phosphorylation of the microtubule-stabilizing protein Tau was observed in the hippocampus (FIGS. 13A-13B) of WD-domain deficient (Atg16L^{ΔWD}) mice with pronounced accumulation in the CA3-field (FIG. 13C) as well as throughout the brain (FIGS. 13D-13F). Proline directed kinase (PDK)-dependent phosphorylation of Tau at serine residues 199 and 202 (S199/S202) as observed in the WD-domain deficient aged mice is highly correlative to Tau phosphorylation observed in human AD brain (PMID: 30016458). The S202 phosphorylation is well characterized as a major contributing phosphorylation event in the development of human neurofibrillary tangles, defined as aggregates of hyperphosphorylated Tau and is a known disease relevant epitope leading to synaptic and neuronal dysfunction. Moreover, it was noted that the phosphorylation present in the WD-domain deficient (Atg16L^{ΔWD}) mice is on endogenous Tau, driven entirely by the sole genetic manipulation of the Atg16L WD-domain. These findings in Tau pathology are therefore fully independent of either ectopic or overexpression of human Tau or mutants of human Tau, models frequently used to study these and other Tau phosphorylation events and associated physiological consequences.

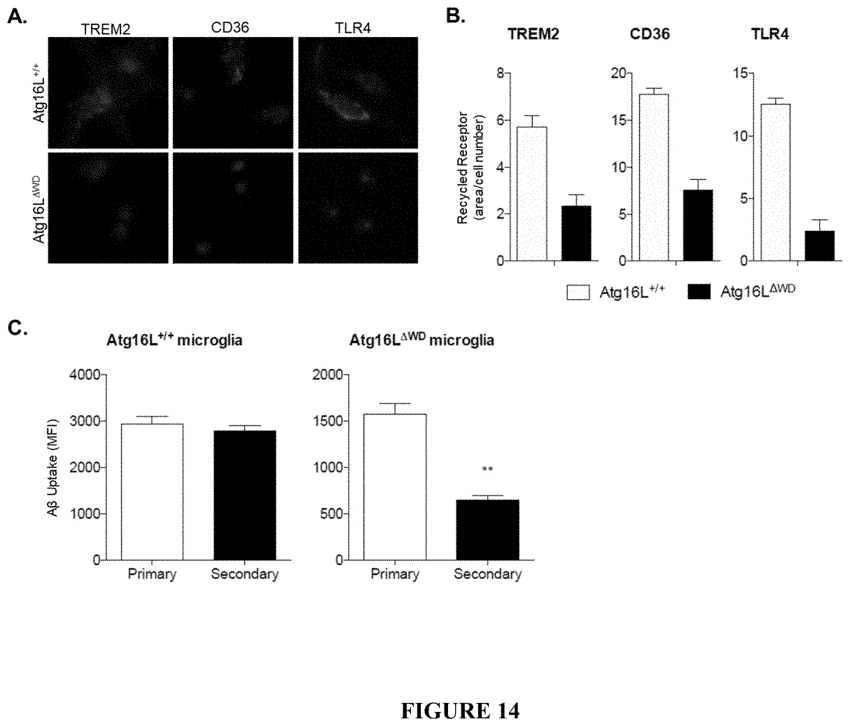

Since aged mice lacking the WD-domain of Atg16L had robust Aβ deposition and Tau hyperphosphorylation, it was important to know how the loss of the WD-domain was contributing to endogenous Aβ accumulation. It has previously been shown that components of the autophagy machinery are required for recycling of Aβ receptors in LANDO, and defects in this recycling can lead to aberrant Aβ accumulation. Therefore, to interrogate a plausible mechanism leading to Aβ deposition, LANDO-dependent recycling of the putative Aβ receptors TREM2, CD36, and TLR4, and contribution of the WD-domain of Atg16L to this process was evaluated. As described in FIGS. 14A-14B, recycling of all three receptors was found to be contingent on the WD-domain of Atg16L in primary microglial cells, consistent with a putative role for this domain in the LANDO pathway. This impairment in LANDO-dependent recycling led to decreased secondary uptake of Aβ in primary microglia lacking the WD-domain of Atg16L (FIG. 14C). Thereby, failed Aβ clearance through loss of LANDO could be contributing to the observed accumulation in vivo. It is important to note however, that the WD-domain of Atg16L has confirmed roles in other pathways marked by LC3-lipidation at single membranes, including, xenophagy and LC3-associated phagocytosis. Therefore, it cannot be fully delineated that the abrogation of LANDO by deletion of the Atg16L WD-domain is exclusively responsible for the deposition of Aβ observed in the aged Atg16L^{ΔWD} mice. However, taken together, these data show that full length Atg16L is required for LANDO-dependent recycling of Aβ receptors in microglia and loss of the WD-domain of Atg16L is sufficient to drive AD-like pathology of endogenous murine Aβ and Tau.

### Example 8. Mice Lacking the WD-Domain of Atg16L have Robust Neuroinflammation

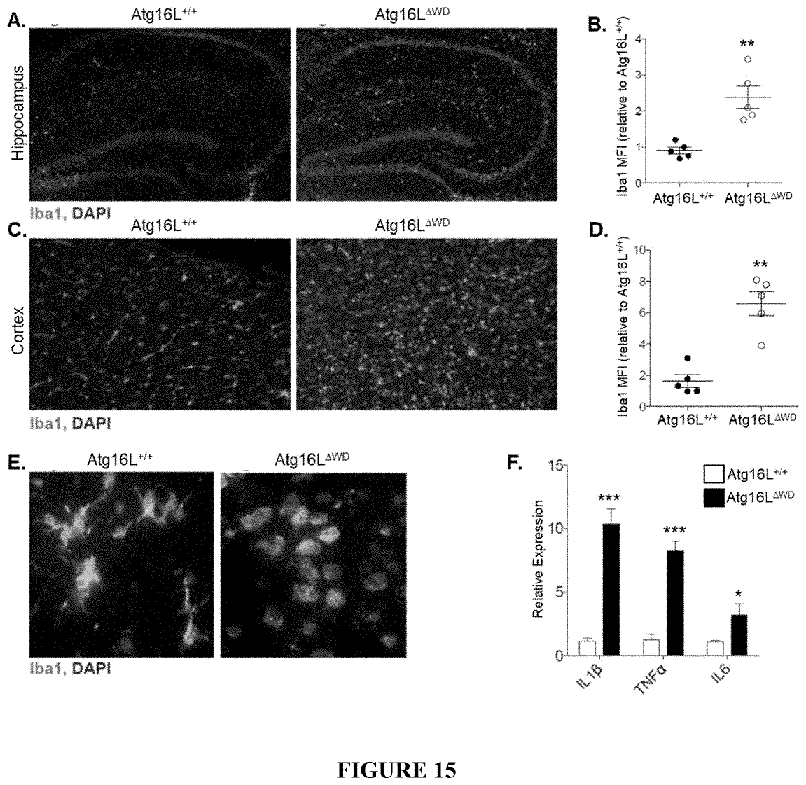

As described in Example 7, mice lacking the WD-domain of Atg16L (Atg16L^{ΔWD} mice) showed spontaneous AD-like pathology, including robust deposition of endogenous Aβ. A consequence of Aβ-deposition in both human disease and murine models of AD is the activation of microglia towards a pro-inflammatory phenotype. Thus, to determine the effect of WD-domain deficiency on neuroinflammation, aged mice lacking the WD-domain of Atg16L (Atg16L^{ΔWD} mice) were evaluated. The findings are described in FIGS. 15A-15F.

As described in FIGS. 15A-15B, an exacerbated activation of microglia was observed in the hippocampi of mice lacking the WD-domain of Atg16L (Atg16L^{ΔWD} mice) compared to wild-type littermates (Atg16L^{+/+} mice) at 2 years of age. Similar to observations regarding Aβ-deposition and phospho-Tau (described in Example 7), microglial activation was not restricted to the hippocampus and was prevalent throughout the cerebral cortex (FIGS. 15C-15D). Moreover, in addition to upregulation of Iba1, microglia in aged Atg16L^{ΔWD} mice showed a transition from ramified to ameboid morphology (FIG. 15C), consistent with inflammatory polarization. Next, the level of pro-inflammatory cytokines IL10, IL6, and TNFα was evaluated in aged mice lacking the WD-domain of Atg16L (Atg16L^{ΔWD} mice), as these inflammatory mediators are often elevated in brains of AD patients and are known to be major components in disease progression. As described in FIG. 15F, when compared to littermate controls (Atg16L^{+/+} mice), mice lacking the Atg16L WD-domain (Atg16L^{ΔWD} mice) showed increased neuroinflammation, which was evident from increased expression of IL1P, IL6, and TNFα in the hippocampus, again paralleling healthy vs AD-pathology in humans.

### Example 9. Atg16L WD-Domain Deficiency Leads to Neurodegeneration in Aged Mice

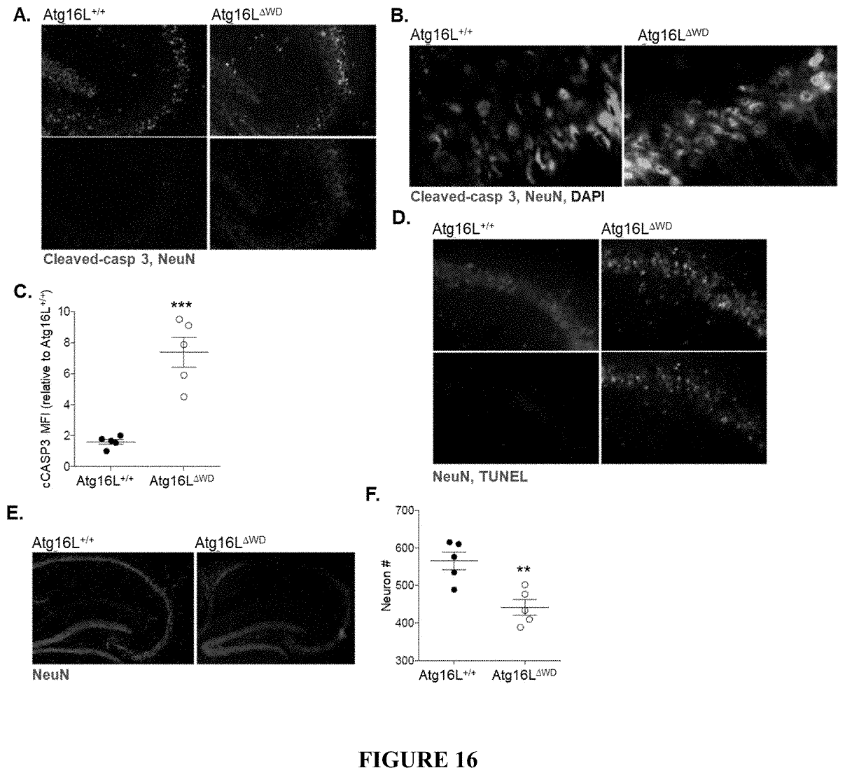

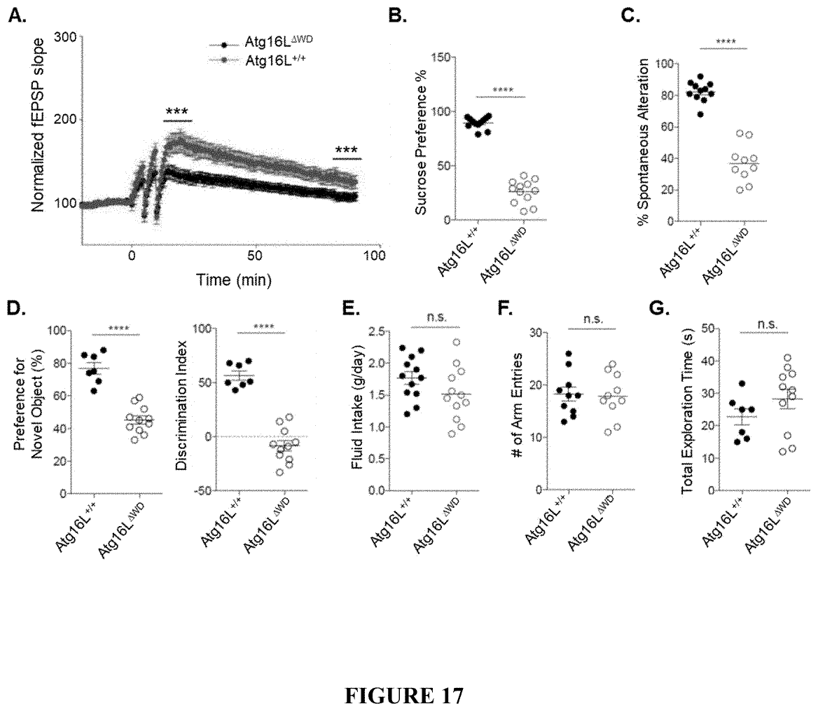

As described in Examples 7 and 8, mice lacking the WD-domain of Atg16L (Atg16L^{ΔWD} mice) showed endogenous Aβ-deposition, Tau phosphorylation, and neuroinflammation, all of which are known risk factors for impairment of neuronal function. Thus, to determine the effect of WD-domain deficiency on impairment of neuronal function, neuronal architecture and function of Atg16L WD-domain deficient mice (Atg16L^{ΔWD} mice) was evaluated. The findings are described in FIGS. 16A-16F and 17A-17G.

Compared to control littermates (Atg16L^{+/+} mice), 2-year-old Atg16L^{ΔWD} mice had widespread cleavage of caspase 3 in CA3-pyramidal neurons extending into the axonal protrusions, suggesting activation of caspase-3 and apoptotic death (FIGS. 16A-16C). To confirm these findings and to interrogate the status of cell death in the CA3-field more intimately, TUNEL-staining was used as a secondary cell death analytic. Consistent with cleavage of caspase 3, 2-year-old Atg16L^{ΔWD} mice showed an increase in TUNEL positive neurons in the CA3-field compared to wild-type (Atg16L^{+/+}) mice (FIG. 16D), suggesting active neurodegeneration. Moreover, but not surprisingly, 2-year-old Atg16L^{ΔWD} mice had a reduction in total neurons within the hippocampus, quantified using the neuronal nuclei marker NeuN (FIGS. 16E-16F). Together, these data suggest that aged mice lacking the WD-domain of Atg16L have an increased susceptibility for neuronal death in the presence of Aβ-induced neuroinflammation.

Due to the observed loss of neurons within the CA3-field, the physiological function of neurons within the hippocampus was next evaluated. Impaired synaptic plasticity is a well-established consequence of AD in humans. Using hippocampal electrophysiology, it was found that 2-year-old Atg16L^{ΔWD} mice had a significant reduction in hippocampal long-term potentiation (LTP) (FIG. 17A). While the deposition of neurotoxic Aβ peptides has long been linked to impairments in synaptic plasticity in humans recent evidence suggests that soluble, non-fibrillary or plaque bound Aβ species are exponentially more detrimental to neuronal function as measured by impaired LTP. Interestingly, the findings disclosed herein regarding Aβ deposition in non-plaque like structures and the effects observed on LTP in the aged mice are strikingly similar to what has been shown in human AD brain samples (PMID: 30409172).

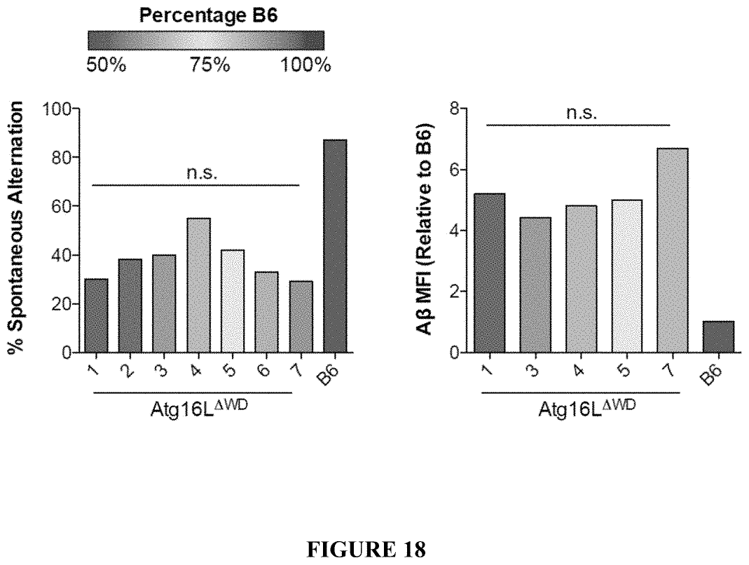

As a consequence of reduced LTP, Atg16L^{ΔWD} mice presented with severe behavioral and memory deficiency. As described in FIGS. 17B-17D, sucrose preference (SPT), spontaneous alternation (Y-maze), and novel object recognition (NOR) were all drastically impaired in 2-year-old mice lacking the WD-domain of Atg16L (Atg16L^{ΔWD} mice) when compared to littermate controls (Atg16L^{+/+} mice). There were no measurable differences in either fluid intake or total number (#) of arm entries for Atg16L^{ΔWD} mice compared to wild-type (Atg16L^{+/+}) mice for the SPT and Y-maze, respectively (FIGS. 17E-17F). Interestingly, Atg16L^{ΔWD} mice did have an increase in total exploration time during the NOR (FIG. 17G). Although this data is not significant, it is consistent with previous reports in both mice and humans with AD, where exploration time is typically increased to offset the inability to discern either the object (mice) or the locale (human). Moreover, the deficiencies in behavior and memory were independent of background strain. The Atg16L WD-domain deficient mice were produced on a mixed (B6,129) background and were not fully inbred (Rai et al 2018(online) Autophagy 15(4)599-612). Therefore, a SNP background analysis was performed and percentage of background strain was compared to disease markers, including, spontaneous alternation (behavioral) and Aβ burden (pathological). No discernable influence of the prevailing background strain was observed for either marker of disease (FIG. 18). As a whole, this data demonstrates that loss of the WD-domain of Atg16L and the associated upstream pathology leads to neurodegeneration, dysfunction in synaptic plasticity, and severe behavioral impairment consistent with highly progressive disease.

### Example 10. Inhibition of Neuroinflammation Alleviates AD-Like Pathology in Atg16L^{ΔWD} Mice

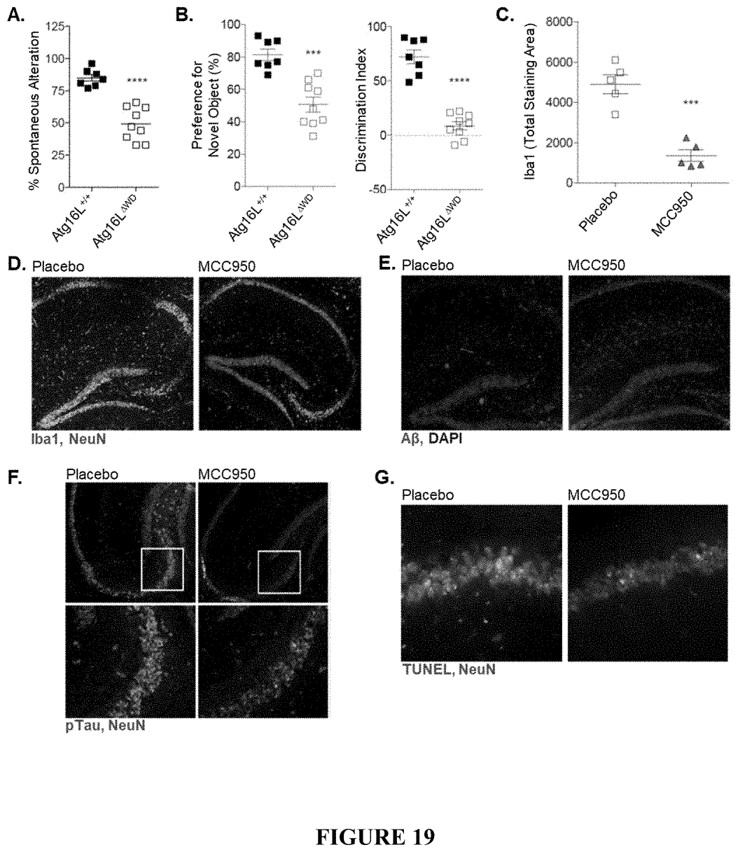

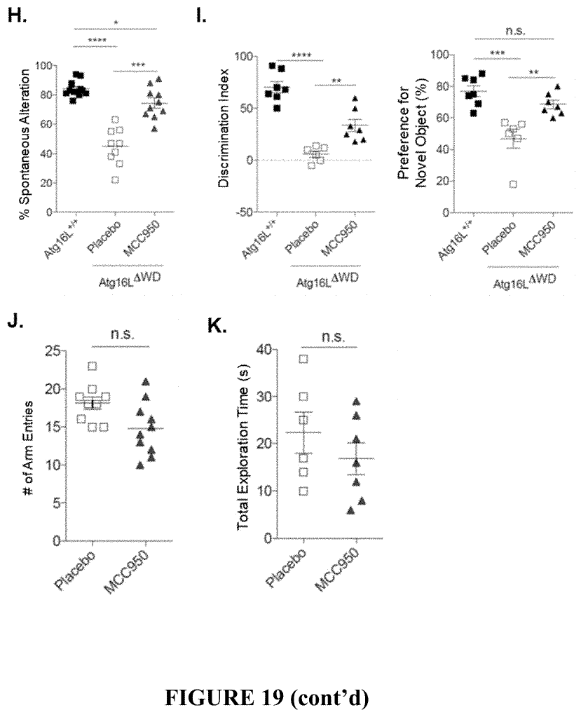

Results described in Examples 7-9 indicated that deletion of the WD-domain of Atg16L leads to age-associated development of an endogenous, spontaneous AD-like pathology. Next, the Atg16L WD-domain deficient mice (Atg16L^{ΔWD} mice) were evaluated to determine whether the observed pathology and memory impairment could be reversed once established, and to what extent was the behavioral pathology a consequence of neurodegeneration compared to neuroinflammation. The findings are described in FIGS. 19A-19K.

Inflammasome inhibition has recently been proposed as putative therapeutic approach that reduces neuroinflammation and Tau phosphorylation (PMID: 31748742). Thus, Atg16L WD-domain deficient mice (Atg16L^{ΔWD} mice) with established disease (starting at 20 months of age) and behavioral impairment, as measured by both impaired spontaneous alternation and NOR (FIGS. 19A-19B), were treated with the brain-penetrant inflammasome inhibitor MCC950. Following 8-weeks of treatment, microglial activation was found to be reduced in Atg16L^{ΔWD} mice treated with MCC950 compared to placebo (FIGS. 19C-19D). However, no difference in Aβ deposition was observed between placebo and MCC950-treated mice (FIG. 19E). Interestingly, inhibition of neuroinflammation resulted in a massive reduction in both Tau phosphorylation (FIG. 19F) and neurodegeneration as measured by TUNEL staining (FIG. 19G).

Consequently, Atg16L^{ΔWD} mice treated with MCC950 had a restoration in their behavioral and memory capacity trending towards wild-type littermates (Atg16L^{+/+} mice) in both the Y-maze (FIG. 19H) and NOR (FIG. 19I) assays, with placebo treated mice continuing to exhibit behavioral decline when compared to treatment onset (FIGS. 19A-19B). No differences in either the # of arm entries or the exploration time were observed between the placebo and treated groups (FIGS. 19J-19K). Taken together, these results suggest that neuroinflammation is upstream of Tau phosphorylation and progressive neurodegeneration in AD-pathology and contributes to behavioral deficits beyond those caused by neuronal loss.

### Example 11. Methods for Examples 1-10

### Materials & Reagents

| | | |
|---|---|---|
| REAGENT or RESOURCE | SOURCE | IDENTIFIER |

Antibodies

| | | |
|---|---|---|
| Anti-TLR4 | Abcam | ab22048 |
| Anti-CD36 | Abcam | ab23680 |
| Anti-NeuN (1B7) | Abcam | ab104224 |
| Anti-NeuN | Abcam | ab177487 |
| Anti-LAMP1 | Abcam | ab25630 |
| Anti-CD11b | Abcam | ab8878 |
| Anti-TMEM119 | Abcam | ab209064 |
| Anti-Cleaved-caspase 3 | Cell Signaling | 9664 |
| Anti-FIP200 | Cell Signaling | 12436 |
| Anti-ATG5 | Cell Signaling | 12994 |
| Anti-Rubicon (D9F7) | Cell Signaling | 8465 |
| Anti-Tau | Cell Signaling | 46687 |
| Anti-Ap 82E1 | IBL | 10326 |
| Anti-Iba1 | Novus | NB100-1028 |
| Anti-Aβ MOAB-2 | Novus | NBP2-13075 |
| Anti-TREM2 | R&D Systems | MAB17291 |
| Anti-FLAG M2 | Sigma-Aldrich | F3165 |
| Anti-β-actin HRP | Thermo | MA1-91399 |
| Anti-phospho-tau (S202/T205) | Thermo | 44-768G |

Chemicals, Peptides, and Misc, Reagents

| | | |
|---|---|---|
| p-amyloid (1-42) peptide - unlabeled | Anaspec | AS-20276 |
| p-amyloid (1-42) peptide - TAMRA | Anaspec | AS-60476 |
| p-amyloid (1-42) peptide - Hilyte 488 | Anaspec | AS-60479-01 |
| p-amyloid (1-42) scrambled peptide - TAMRA | Anaspec | Custom Synthesis |
| Dextran - Texas Red | Invitrogen | D1863 |
| Zymosan A - AF594 | Invitrogen | Z23374 |
| ProLong Diamond DAPI mounting media | Invitrogen | P36971 |
| Rapamycin | Sigma-Aldrich | R8781 |
| Latrunculin A | Sigma-Aldrich | L5163 |

Commercial Assays & Kits

| | | |
|---|---|---|
| Neural Tissue Dissociation Kit | Miltenyi | 130-092-628 |
| Microglia Isolation Kit | Miltenyi | 130-093-634 |
| Universal SYBR Green | Bio-Rad | 1725271 |
| M-MLV Kit | Invitrogen | 28025013 |
| CLICK-IT™ TUNEL Alexa Fluor™ 488 Imaging | Invitrogen | C10245 |
| Assay kit | | |
| Mouse Multianalyte Inflammatory Cytokine ELISA | Qiagen | MEM-004A |
| RNeasy Mini Kit | Qiagen | 74104 |

Oligonucleotides

| | | |
|---|---|---|
| Rubicon-sgRNA1 | N/A | N/A |
| 5′-CACCGAGGAGACTCGTCCATACACG-3′ | | |
| (SEQ ID NO: 1) | | |
| 3′-AAACCGTGTATGGACGAGTCTCCTC-5′ | | |
| (SEQ ID NO: 2) | | |
| Rubicon-sgRNA2 | N/A | N/A |
| 5′-CACCGTGATGAGGAACGGGCGAAGA-3′ | | |
| (SEQ ID NO: 3) | | |
| 3′-AAACTCTTCGCCCGTTCCTCATCAC-5′ | | |
| (SEQ ID NO: 4) | | |
| ATG5-sgRNA1 | N/A | N/A |
| 5′-GTGAGCCTCAACCGCATCCT-3′ (SEQ ID | | |
| NO: 5) | | |
| 3′-CACTCGGAGTTGGCGTAGGA-5′ (SEQ ID | | |
| NO: 6) | | |
| ATG5-sgRNA2 | N/A | N/A |
| 5′-CGGAACAGCTTCTGGATGAA-3′ (SEQ ID | | |
| NO: 7) | | |
| 3′-GCCTTGTCGAAGACCTACTT-5′ (SEQ ID | | |
| NO: 8) | | |
| FIP200-sgRNA1 | N/A | N/A |
| 5′-AGAGTGTGTACTTACAGCGC-3′ (SEQ ID | | |
| NO: 9) | | |
| 3′-TCTCACACATGAATGTCGCG-5′ (SEQ ID | | |
| NO: 10) | | |
| FIP200-sgRNA2 | N/A | N/A |
| 5′-GAGGATCATGCTCCTAGAAC-3′ (SEQ ID | | |
| NO: 11) | | |
| 3′-CTCCTAGTACGAGGATCTTG-5′ (SEQ ID | | |
| NO: 12) | | |
| Actin qPCR | N/A | N/A |
| F: ATGGAGGGGAATACAGCCC (SEQ ID NO: | | |
| 13) | | |
| R: TTCTTTGCAGCTCCTTCGTT (SEQ ID NO: | | |
| 14) | | |
| TNFa qPCR | N/A | N/A |
| F: CCTGTAGCCCACGTCGTAGC (SEQ ID NO: | | |
| 15) | | |
| R: AGCAATGACTCCAAAGTAGACC (SEQ ID | | |
| NO: 16) | | |
| IL1b qPCR | N/A | N/A |
| F: CACAGCAGCACATCAACAAG (SEQ ID NO: | | |
| 17) | | |
| R: GTGCTCATGTCCTCATCCTG (SEQ ID NO: | | |
| 18) | | |
| IL6 qPCR | N/A | N/A |
| F: GAGGATACCACTCCCAACAGACC (SEQ ID | | |
| NO: 19) | | |
| R: AAGTGCATCATCGTTGTTCATACA (SEQ ID | | |
| NO: 20) | | |
| CCL5 qPCR | N/A | N/A |
| F: CCAATCTTGCAGTCGTGTTTGT (SEQ ID NO: | | |
| 21) | | |
| R: CATCTCCAAATAGTTGATGTATTCTTGAAC | | |
| (SEQ ID NO: 22) | | |
| Iba1 qPCR | N/A | N/A |
| F: CAGACTGCCAGCCTAAGACA (SEQ ID NO: | | |
| 23) | | |
| R: AGGAATTGCTTGTTGATCCC (SEQ ID NO: | | |
| 24) | | |

### Experimental Model & Subject Details

### Mice

The 5×FAD transgenic mice carrying the following five mutations: Swedish (K670N and M671L), Florida (I716V) and London (V717I) in human APP695 and human PS1 cDNA (M146L and L286V) under the transcriptional control of the neuron-specific Thy-1 promoter and were purchased from The Jackson Laboratory. 5×FAD mice were crossed to FIP200^{fl/fl} LysM-Cre+ (kindly provided by Jun-Lin Guan, University of Michigan), ATG5^{fl/fl} LysM-Cre+ (kindly provided by Thomas A. Ferguson, Washington University), and Rubicon^{-/-} mice which were generated as described previously (Martinez et al. (2015) Nat Cell Biol 17:893-906). Mice used for bone marrow isolation and BMDM culture were kindly provided as follows; Beclin1^{fl/fl} LysM-Cre+ (Edmund Rucker, University of Kentucky), ATG7^{fl/fl} LysM-Cre+ (Masaaki Komatsu at The Tokyo Metropolitan Institute of Medical Science), ATG14^{fl/fl} LysM-Cre+ (Herbert Virgin, Washington University), VPS34^{fl/fl} LysM-Cre+ (Richard Flavell, Yale University), and ULK1^{-/-} LysM-Cre+ (Mondira Kundu, St. Jude Children's Research Hospital).

Unless otherwise noted, all experiments were performed on mixed sex cohorts at 4-months of age. Depending on genotype, either LysM-cre⁻ or Rubicon^{+/-} littermates were used as controls.

Atg16^{ΔWD} mice were generated by deletion of the WD-domain of Atg16L, as previously described (Rai et al 2018(online) Autophagy 15(4)599-612). In brief, 2 stop codons were inserted into exon 6 of murine Atg16L1 immediately after glutamate E230 to preserve binding sites for WIPI2 (required for canonical autophagy) but prevent translation of the linker and WD-domain. Mice were produced and maintained on a mixed 129, C57BL/6 background. Unless otherwise noted, all experiments were performed on mixed sex cohorts at 2 years of age. Mice treated with MCC950 or placebo were mixed sex cohorts, 20 months of age at time of treatment onset and were treated for 8-weeks. The genetic backgrounds of mice used were assessed at the DartMouse™ Speed Congenic Core Facility at the Geisel School of Medicine at Dartmouth. DartMouse uses the Illumina, Inc. (San Diego, Calif.) Infinium Genotyping Assay to interrogate a custom panel of 5307 SNPs spread throughout the genome. The raw SNP data was analyzed using DartMouse's SNaP-Map™ and Map-Synth™ software, allowing the determination for each mouse of the genetic background at each SNP location. Background strain percentage was subsequently compared against markers of disease pathology to evaluate any influence stemming from variations in background (FIG. 18).

The St. Jude Institutional Animal Care and Use Committee approved all procedures in accordance with the Guide for the Care and Use of Animals. All mice were housed in pathogen-free facilities, in a 12-hour light/dark cycle in ventilated cages, with chow and water supply ad libitum.

### Cells

BV2 murine microglia and RAW264.7 cells were obtained from ATCC. Cells were maintained in complete DMEM media (10% fetal bovine serum (FBS), 200 mM L-glutamine and 100 units/ml penicillin-streptomycin). All the cell lines used were confirmed as mycoplasma negative using MycoAlert Mycoplasma Detection kit (Lonza #LT07).

For preparation of bone marrow-derived macrophages (BMDM), male or female mice at 6 to 12 weeks of age were euthanized and bone marrow cells were harvested from the femurs and differentiated in DMEM containing 20% FBS, 200 mM L-glutamine, 100 units/ml penicillin-streptomycin, 20 ng/ml recombinant human M-CSF for 10 days. BMDMs were harvested and seeded on tissue culture plates one day before stimulation and maintained in complete DMEM media. All cells used in this study were cultivated at 37° C. with 5% CO2.

### Method Details

### Generation and Maintenance of Cell Lines

BV2 microglia deficient in FIP200, ATG5, and Rubicon were generated using CRISPR/Cas9 technology by lentiviral transduction and puromycin selection. Two guide RNAs (gRNA) were designed for each gene (See Reagents List for sequences) and cloned into the pLenti-V2 plasmid (Addgene). Lentivirus was produced using HEK293T cells co-expressing pPAX and pVSVg plasmids (Addgene) and our CRISPR pLenti-V2 plasmids using Lipofectamine 2000 (Invitrogen). BV2 cells were subsequently transduced and transduction efficiency was confirmed by immunoblot analysis following two weeks of puromycin selection. An empty pLenti-V2 vector was transduced to establish a parental cell line. Once confirmed, cells were then exposed to LentiBrite GFP-tagged LC3 lentivirus (Millipore) to establish GFP-LC3 positive lines.

RAW264.7 lines deficient in Rubicon or ATG5 were established using CRISPR/Cas9 viral transduction as described above for BV2 cells. RAW264.7 cells that overexpress either RavZ or a dominant-negative ATG4 were generated by transduction using a retrovirus carrying pMXs-Flag-mATG4B-C74A (Blasticidin) or pMXs-Flag-RavZ (Blasticidin) or the empty vector. The retroviral vectors were created as follows. Mouse ATG4B was cloned from a mouse cDNA library into pMXs retroviral vector. The active site Cysteine (C74) was mutated to Alanine using site-directed mutagenesis. The original vector expressing RavZ was a gift from Craig Roy (Yale University), the ORF was subcloned into pMXs.

All lentiviral and retroviral work was performed in accordance with the guidelines set forth by the SJCRH Institutional Biosafety Committee and within the scope of our approved Biosafety protocol.

### β-Amyloid Preparation and Treatment

Both labeled and unlabeled Ab1-42 was purchased in lyophilized form and resuspended according to the manufacturer's recommendation at a concentration of 100 μM (Anaspec). In brief, Ab1-42 was resuspended to 5 mM in DMSO and then adjusted to 100 μM using DMEM/F12 culture media. Oligomerization was allowed to occur for 24h at 4° C. prior to addition to cells at 1 μM unless otherwise indicated.

### Primary Microglia Isolation and Culture

Mice were anesthetized with isoflurane and perfused with 1% BSA in PBS. Brains were subsequently harvested and immediately processed using the papain-based Neural Dissociation Kit (Miltenyi). Myelin was removed using myelin removal beads and microglia were purified using CD11b microglia beads (Miltenyi). Isolated cells were subsequently cultured and maintained in complete DMEM media (10% fetal bovine serum (FBS), 200 mM L-glutamine and 100 units/ml penicillin-streptomycin) at 37° C. with 5% CO2. The cells were then used as indicated. All steps were performed per the manufacturer's instructions.

### Microscopy and Image Analysis

For all non-live cell-based imaging, cells were cultured in 4-well chamber slides (Ibidi) and were fixed and stained as indicated. In brief, cells were fixed with 4% PFA for 10 min followed by permeabilization using 200 μg/ml digitonin for 10 min. Cells were blocked in 0.5% BSA in PBS for 30 min prior to staining with primary antibodies overnight at 4° C. Cells were then washed 3× in PBS and then stained with the indicated fluorescent secondary antibodies for 30 min. Cells were subsequently washed 3× with PBS and post-fixed in 1% PFA for 10 min prior to imaging. For all live cell-based imaging, cells were immediately transferred to an environment controlled, live-cell imaging chamber (Ibidi).

For preparation of brain tissue see “Preparation of brain samples” below. Slides were subjected to antigen retrieval using 1% sodium citrate boiling for 20 min followed by 3× PBS washing. Slides were blocked in 0.5% BSA in PBS. Antibody staining was carried out as described above. Following final washing, slides were mounted using ProLong Diamond Anti-Fade mounting media with DAPI.

All imaging was performed on either an Eclipse Ti-E TIRF/N-Storm/epifluorescence microscope (Nikon) or a MARIANIS spinning disk confocal microscope (Intelligent Imaging Innovations (3i)) equipped with an EMCCD camera. Image analysis including all quantification was performed using Nikon NIS-elements Advanced Research Imaging software or Slidebook 6 (3i).

Image analysis for relative Aβ, Iba1, and phospho-Tau staining was achieved by quantifying the mean fluorescent intensity (MFI) of either Iba1 or phospho-Tau signal using NIS-elements. Analysis and quantification of microglial morphology was achieved using Slidebook 6 software. Morphological state was determined by measuring cell diameter following 3D reconstruction and confirmed by manual counting/analysis of microglia shape per defined field across multiple areas of each slide.

### Flow Cytometry

For all uptake assays, cells were analyzed without fixation. For membrane-associated GFP-LC3 analysis, cells were processed as described below. For brain infiltrating monocytes, cells were isolated as described using the Neural Tissue Dissociation Kit (Miltenyi). Primary cells were fixed, permeabilized, and stained using the Cyto Fix/Perm Staining Kit (BD Bioscience) and the indicated, conjugated primary antibodies. For all experiments, cells were analyzed using a Sony SP6800 Spectral Analyzer (Sony). All analyses were performed using FlowJo v10.4 (Tree Star). Fluorescent compensation was performed using BD compensation beads (BD Bioscience).

### Preparation of Brain Samples

Mice were anesthetized with isoflurane and perfused with ice-cold PBS containing 1 U/ml of heparin. Right brain hemispheres were fixed in 4% PFA overnight at 4° C., rinsed in PBS, and incubated overnight at 4° C. in 30% sucrose before freezing in a 2:1 mixture of 30% sucrose and optimal cutting temperature compound (OCT). Serial 20 μm coronal sections were cut on a cryo-sliding microtome. Cortices and hippocampi of the left-brain hemispheres were carefully dissected out and flash frozen for biochemical analysis or processed for RNA isolation.

### Membrane-Associated LC3 Analysis

To quantify membrane association of GFP-LC3, cells were harvested and permeabilized using 200 μg/ml digitonin for 15 min on ice. Cytosolic GFP-LC3 was removed by washing cells 5× in cold PBS. Cells were then resuspended in 0.5% BSA in PBS for analysis by flow cytometry as described above.

### Cell & Tissue Lysis and Immunoblot

Cells were lysed in RIPA buffer for 30 min on ice [50 mM Tris (pH 7.5), 150 mM NaCl, 1% Triton X100, 0.5% deoxycholate (DOC), 0.1% SDS, protease inhibitor tablet (Roche), 1 mM NaF, 1 mM Na₃VO₄, and 1 mM PMSF]. Brain samples were mechanically homogenized in RIPA buffer. After centrifugation, supernatants were analyzed by SDS/PAGE. All blots were imaged using H1RP-conjugated secondary antibodies and ECL using a LiCOR Odyssey Fx imaging system (LiCOR). All immunoblot analysis was performed using LiCOR Image Studio software.

### Real-Time RT-PCR

Total RNA was isolated from cells or tissue using the RNeasy Kit (Qiagen) according to the manufacturer's instructions. First-strand synthesis was performed using M-MLV reverse transcriptase (Invitrogen). Realtime PCR was performed using SYBR GREEN PCR master mix (Applied Biosystems) in an Applied Biosystems 7900HT thermocycler using SyBr Green detection protocol as outlined by the manufacturer using the following PCR conditions: 50° C. for 2 min, 95° C. for 10 min, and 40 cycles of 95° C. for 15s and 60° C. for 1 min. mRNA was normalized to actin allowing for comparison of mRNA levels. Please see key reagents table for qPCR primer sequences.

### Receptor Recycling

For receptor recycling, cells were plated on 4-well Ibidi tissue culture-coated chamber slides and allowed to reach 50% confluence. Cells were then blocked for 15 min in the presence of 10% normal donkey-serum at 37° C. Primary antibodies targeting the indicated receptor (see reagent list) were then added at a dilution of 1:100 in 1% donkey-serum in DMEM and cells were incubated at 37° C. for 1h. Antibody-containing media was aspirated and cells were acid washed with cold-DMEM, pH 2.0. Cells were returned to 10% donkey-serum in DMEM for 1 h. Alexa Fluor 568-labeled secondary antibodies were diluted 1:1000 in 1% donkey-serum in DMEM and added to cells for 1 h at 37° C. to label recycled receptors. Cells were subsequently acid washed as described above and then fixed in 4% PFA in PBS for 15 min. Cell permeable Hoechst dye was added to label nuclei.

Quantification of recycling was achieved by calculating the sum of AF568-fluorescent area divided by the total number of cells. Nikon NIS-Elements AR software was used for all image analyses and quantification.

### Amyloid Uptake

Primary and secondary β-amyloid uptake was assayed as follows. BV2 clones were treated with 1 μM Alexa Fluor 488-labeled Aβ1-42. Mean fluorescent intensity (MFI) for AF-488 was determined by flow cytometry after 12h and considered the primary uptake. 1 μM TAMRA-labeled Aβ1-42 was subsequently added to the medium following the primary uptake phase. MFI for TAMRA was assessed by flow cytometry 12h following the primary uptake timepoint. This timepoint constitutes the secondary uptake.

### Phagocytosis and Endocytosis Analysis

To delineate between phagocytosis and endocytosis, cells were treated as indicated with the phagocytic inhibitor latrunculin A. Cells were pre-treated for 1 h prior to the addition of target substrates. The following control substrates were used, zymosan (phagocytosis) and dextran (endocytosis), both were fluorescently labeled as indicated. Co-incubation with specific substrates was carried out at 37° C. for 3h. Cells were either fixed for imaging or analyzed by flow cytometry as described above respectively.

### Electrophysiology

Acute transverse hippocampal slices (400 m) were prepared as previously described (Gingras et al. (2015) J Neurosci 35:10510-10522). Briefly, mouse brains were quickly removed and placed in cold (4° C.) dissecting ACSF containing 125 mm choline-Cl, 2.5 mm KCl, 0.4 mm CaCl₂), 6 mm MgCl₂, 1.25 mm NaH₂PO₄, 26 mm NaHCO₃, and 20 mm glucose (285-295 mOsm) under 95% O2 and 5% CO2. After dissection, slices were incubated for 1 h in ACSF containing 125 mm NaCl, 2.5 mm KCl, 2 mm CaCl₂), 2 mm MgCl₂, 1.25 mm NaH₂PO₄, 26 mm NaHCO₃, and 10 mm glucose (285-295 mOsm) under 95% O2 and 5% CO2 at room temperature and then transferred into the submerged recording chamber and superfused (2-3 ml/min) with warm (30° C.-32° C.) ACSF. The field recordings were performed by using a setup with 8 submerged recording chambers (Campden Instruments). The fEPSPs were recorded from the CA1 stratum radiatum by using an extracellular glass pipette (3-5 MΩ) filled with ACSF. Schaffer collateral/commissural fibers in the stratum radiatum were stimulated with a bipolar tungsten electrode placed 200-300 m away from the recording pipette.

### Behavior & Memory Analysis

For sucrose preference tests (SPT), mice were individually housed and allowed to acclimate to the testing room for 48h prior to starting the experiment. A dual bottle setup was introduced where both bottles contained only standard water. Again, mice were allowed to acclimate to the dual bottle setup for 3 days. After acclimation, one bottle was replaced with a 2% sucrose solution. Water consumption was monitored daily for 4 days. Bottles were rotated daily to minimize side bias and normalized for leakage. All results are shown as the averaged consumption and preference over the 4-day test period.

For Y-maze spontaneous alternation analysis, mice were housed in the testing room and allowed to acclimate for 48h. The Y-maze test consisted of a single 5 min trial per mouse. Spontaneous Alternation [%] was defined as consecutive entries in 3 different arms (ABC), divided by the number of possible alternations (total arm entries minus 2). Mice with less than 5 arm entries during the 5 min trial were excluded from the analysis.

Novel object recognition (NOR) was performed in an open-field box (40 cm×40 cm). Mice were allowed to acclimate to the testing room for 48h. For habituation, mice were allowed to explore the open-field for 15 min per day for two days. Mice were then exposed to two identical objects for 10 min on the day of testing. 2h later a novel object was introduced, and mice were allowed to explore for 5 min during the test phase. The time spent exploring each object was quantified manually. Novel object preference (%) and the discrimination index ((time with novel)/(novel+familiar)*100) were calculated for each mouse.

### MCC950 Inflammasome Inhibition In Vivo

Mice with established disease were treated for 8-weeks with either a vehicle (placebo) control or MCC950 (Invivogen) as reported previously (Gordon et al. 2018 Science Translational Medicine 10(465)). In brief, MCC950 was suspended in 100% DMSO and titrated to a working dose using sterile water. The final concentration of DMSO in the injection was <1%. Matching solution without MCC950 was used as the vehicle (placebo). Mice were injected every 3 days for 8-weeks at a dose of 10 mg/kg via intraperitoneal injection.

### Statistical Analysis

Please refer to the descriptions of the figures for description of sample sizes and statistical test performed. Data were plotted and analyzed with GraphPad Prism 7.0 software. All experiments were designed and are powered to a minimum of 0.8 as calculated using G*Power. Differences were considered statistically significant when the p-value was less than 0.05.

We claim:

1. A method for decreasing neuroinflammation or neurodegeneration in a LC3-associated endocytosis (LANDO)-deficient subject comprising administering an effective amount of a pharmaceutical composition that activates or enhances the LANDO pathway, wherein said administration of an effective amount of a pharmaceutical composition that activates or enhances the LANDO pathway decreases neuroinflammation or neurodegeneration.

2. The method of claim 1, wherein said pharmaceutical composition that activates or enhances the LANDO pathway has no significant effect on LC3-associated phagocytosis (LAP).

3. The method of claim 1 or 2, wherein said LANDO-deficient subject has reduced expression of at least one of: Beclin1, VPS34, ATG5, ATG7, ATG4, LC3A, LC3B, Rubicon, and Atg16L WD-domain; when compared to a subject not deficient in LANDO.

4. The method of any one of claims 1-3, wherein said LANDO-deficient subject has reduced expression of Rubicon, ATG5 or Atg16L WD-domain when compared to a subject not deficient in LANDO.

5. The method of any one of claims 1-4, further comprising detecting failed clearance of β-amyloid prior to administering an effective amount of said pharmaceutical composition.

6. The method of any one of claims 1-5, wherein said decreased neuroinflammation or neurodegeneration comprises any one of: reduced expression of pro-inflammatory genes, reduced β-amyloid deposition or plaque formation, reduced tau hyperphosphorylation, reduced microglial activation, reduced microglial ramified to ameboid transition, reduced microgliosis, reduced neuronal cell death, reduced electrophysiological impairment, reduced behavior deficits, and reduced memory deficits.

7. A method for treating Alzheimer's disease comprising administering an effective amount of a pharmaceutical composition that activates or enhances the LANDO pathway to a subject diagnosed with Alzheimer's disease or demonstrating symptoms of the disease, wherein said administration of an effective amount of a pharmaceutical composition that activates or enhances the LANDO pathway decreases at least one symptom of Alzheimer's disease.

8. The method of claim 7, wherein said subject has reduced expression of at least one of: Beclin1, VPS34, ATG5, ATG7, ATG4, LC3A, LC3B, Rubicon, and Atg16L WD-domain; when compared to a subject not deficient in LANDO.

9. The method of claim 7 or 8, wherein said subject has reduced expression of Rubicon or ATG5 when compared to a subject not deficient in LANDO.

10. The method of any one of claims 7-9, further comprising detecting failed clearance of β-amyloid prior to administering an effective amount of said pharmaceutical composition.

11. A method for clearing β-amyloid in a subject deficient in β-amyloid clearance comprising administering an effective amount of a pharmaceutical composition that activates or enhances the LANDO pathway.

12. The method of claim 11, wherein said subject is a LANDO-deficient subject.

13. The method of claim 12, wherein said subject has reduced expression of at least one of: Beclin1, VPS34, ATG5, ATG7, ATG4, LC3A, LC3B, Rubicon, and Atg16L WD-domain; when compared to a subject not deficient in LANDO.

14. The method of claim 12 or 13, wherein said subject has reduced expression of Rubicon or ATG5 when compared to a subject not deficient in LANDO.

15. The method of any one of claims 11-14, wherein said subject comprises β-amyloid accumulation in at least one of the cortex and hippocampus prior to administration of said pharmaceutical composition.

16. The method of claim 15, wherein said subject exhibits symptoms of said β-amyloid accumulation prior to administration of said pharmaceutical composition.

17. A method for identifying a compound that modulates LANDO activity and does not significantly modulate LAP activity, said method comprising:
measuring a first level of LANDO activity and LAP activity in a cell or tissue;
contacting the cell or tissue with a candidate compound;
measuring a second level of LANDO activity and LAP activity of said cell or tissue after contact with said candidate compound;
comparing said first level of LANDO activity with the second level of LANDO activity and comparing said first level of LAP activity with the second level of LAP activity; and
selecting compounds that modulate the LANDO activity and do not significantly modulate the LAP activity.

18. A method for identifying a compound that modulates LANDO activity and does not significantly modulate LAP activity, said method comprising:
contacting a test cell or tissue with a candidate compound;
measuring a first level of LANDO activity and LAP activity of said test cell or tissue after contact with said candidate compound;
measuring a second level of LANDO activity and LAP activity from a control cell or tissue;
comparing said first level of LANDO activity with said second level of LANDO activity and comparing said first level of LAP activity with the second level of LAP activity; and
selecting compounds that modulate the LANDO activity and do not significantly modulate the LAP activity.

19. The method of claim 17 or 18, wherein compounds are selected that increase LANDO activity.

20. The method of any one of claims 17-19, wherein measuring said first and second level of LANDO activity comprises measuring β-amyloid clearance.

21. The method of any one of claims 17-20, wherein measuring said first and second level of LANDO activity comprises measuring recycling of at least one β-amyloid receptor from endosomes to plasma membrane.

22. The method of claim 21, wherein said at least one β-amyloid receptor is selected from CD36, TLR4, and TREM2.

23. The method of any one of claims 17-22, wherein measuring said first and second level of LAP activity comprises measuring phagocytosis.

24. The method of any one of claims 17-23, wherein said cell or tissue comprises a bone marrow-derived macrophage or a culture of bone marrow-derived macrophages, a microglial cell or a culture of microglial cells, or a myeloid cell or a culture of myeloid cells.

25. The method of claim 24, wherein said bone marrow-derived macrophage, microglial cell, or myeloid cell is derived from LANDO-deficient mice.

26. The method of claim 25, wherein said LANDO-deficient mice are Rubicon deficient, ATG5 deficient or Atg16L WD-domain deficient.

27. The method of any one of claims 17-26, wherein said selected molecule modulates LANDO activity when administered to a subject.

28. The method of claim 27, wherein said subject is a LANDO-deficient subject.

29. The method of claim 28, wherein said LANDO-deficient subject has reduced expression of at least one of: Beclin1, VPS34, ATG5, ATG7, ATG4, LC3, Rubicon, and Atg16L WD-domain; when compared to a subject not deficient in LANDO.

30. The method of claim 28 or 29, wherein said LANDO-deficient subject has reduced expression of Rubicon, ATG5 or Atg16L WD-domain when compared to a subject not deficient in LANDO.

31. The method of any one of claims 28-30, wherein said LANDO-deficient subject exhibits neuroinflammation or neurodegeneration.

32. A pharmaceutical composition comprising a molecule selected by the method of any one of claims 17-31.

33. Use of a pharmaceutical composition that activates or enhances the LANDO pathway for decreasing neuroinflammation or neurodegeneration or treating Alzheimer's disease according to the methods of claims 1-6 or 7-10, respectively.

34. Use of a pharmaceutical composition that activates or enhances the LANDO pathway according to the method of any one of claims 1-16 or that is identified by the method of any one of claims 17-30 as a medicament.

35. A pharmaceutical composition that activates or enhances the LANDO pathway for use in treating a neuroinflammatory disorder, neurodegenerative disorder, or Alzheimer's disease in a LANDO-deficient subject, said use comprising administering an effective amount of a pharmaceutical composition that activates or enhances the LANDO pathway to the subject.

36. The pharmaceutical composition of claim 35, wherein said subject has reduced expression of at least one of: Beclin1, VPS34, ATG5, ATG7, ATG4, LC3A, LC3B, Rubicon, and Atg16L WD-domain; when compared to a subject not deficient in LANDO.

37. A mouse model of neuroinflammation or neurodegeneration comprising microglial LANDO knockdown or knockout and at least one additional genetic modification that contributes to neuroinflammation or neurodegeneration.

38. The mouse model of claim 37, wherein said microglial LANDO knockdown or knockout targets at least one of Rubicon, ATG5 and Atg16L WD-domain.

39. The mouse model of claim 37 or 38, wherein said microglial LANDO knockdown or knockout targets Rubicon.

40. The mouse model of any one of claims 37-39, wherein said microglial LANDO knockdown or knockout is tissue-specific.

41. The mouse model of claim 40, wherein said microglial LANDO knockdown or knockout is specific to cells of the myeloid lineage and microglia.

42. The mouse model of any one of claims 37-41, wherein said microglial LANDO knockdown or knockout is mediated by a site-specific recombinase system.

43. The mouse model of claim 42, wherein said site-specific recombinase system comprises Cre/lox.

44. The mouse model of claim 42 or 43, wherein expression of a site-specific recombinase is under the control of the lysozyme 2 promoter.

45. The mouse model of any one of claims 40-44, wherein said knockdown or knockout targets ATG5 or Atg16L WD-domain.

46. The mouse model of any one of claims 37-45, wherein said at least one additional genetic modification that contributes to neuroinflammation or neurodegeneration comprises mutations or expression of transgenic molecules that lead to overexpression of a mutated amyloid precursor protein (APP) present in familial Alzheimer's disease (FAD).

47. The mouse model of claim 46, wherein said mutated amyloid precursor protein comprises at least one of K670N, M671L, I716V, and V717I in relation to human APP(695).

48. The mouse model of claim 47, wherein said mouse model transgenically expresses mutant human APP(695) comprising all of the following mutations: K670N, M671L, I716V, and V717I.

49. The mouse model of claim 48, wherein expression of mutant human APP(695) is regulated by a tissue-specific promoter that is expressed in the central nervous system.

50. The mouse model of claim 49, wherein expression of mutant human APP(695) is under the regulation of the murine Thy1 promoter.

51. The mouse model of any one of claims 46-50, wherein said mouse model transgenically expresses mutant human presinilin 1 comprising a M146L mutation and a L286V mutation.

52. The mouse model of claim 51, wherein expression of mutant human presinilin 1 is regulated by a tissue-specific promoter that is expressed in the central nervous system.

53. The mouse model of claim 52, wherein expression of mutant human presinilin 1 is under the regulation of the murine Thy1 promoter.

54. The mouse model of any one of claims 46-53, wherein said mouse model comprises a 5×FAD transgenic mouse transgenically expressing a mutant human APP(695) with the following mutations: K670N, M671L, I716V, and V717I and transgenically expressing a mutant human presinilin 1 comprising a M146L mutation and a L286V mutation.

55. The mouse model of any one of claims 37-54, wherein said microglial LANDO knockdown or knockout increases penetrance of neuroinflammation or neurodegeneration, reduces age of onset of neuroinflammation or neurodegeneration, or both increases penetrance and reduces age of onset of neuroinflammation or neurodegeneration, when compared to a mouse lacking microglial LANDO knockdown or knockout.

56. A method of making a mouse model of neuroinflammation or neurodegeneration comprising microglial LANDO knockdown or knockout and at least one additional genetic modification that contributes to neuroinflammation or neurodegeneration, wherein said method comprises knocking down or knocking out LANDO in microglial tissues in a mouse comprising at least one additional genetic modification that contributes to neuroinflammation or neurodegeneration.

57. The method of claim 56, wherein said method further comprises introducing said at least one additional genetic modification that contributes to neuroinflammation or neurodegeneration.

58. The method of claim 56, wherein said method comprises crossing a mouse comprising microglial LANDO knockdown or knockout with a mouse comprising at least one additional genetic modification that contributes to neuroinflammation or neurodegeneration.

59. The method of any one of claims 56-58, wherein said microglial LANDO knockdown or knockout targets at least one of Rubicon, ATG5 and Atg16L WD-domain.

60. The method of any one of claims 56-59, wherein said microglial LANDO knockdown or knockout targets Rubicon.

61. The method of any one of claims 56-60, wherein said microglial LANDO knockdown or knockout is tissue-specific.

62. The method of claim 61, wherein said microglial LANDO knockdown or knockout is specific to cells of the myeloid lineage and microglia.

63. The method of any one of claims 56-62, wherein said microglial LANDO knockdown or knockout is mediated by a site-specific recombinase system and wherein said method further comprises generating said mouse comprising microglial LANDO knockdown or knockout using said site-specific recombinase system.

64. The method of claim 63, wherein said site-specific recombinase system comprises Cre/lox.

65. The method of claim 63 or 64, wherein expression of a site-specific recombinase is under the control of the lysozyme 2 promoter.

66. The method of any one of claims 61-65, wherein said knockdown or knockout targets ATG5 or Atg16L WD-domain.

67. The method of any one of claims 56-66, wherein said at least one additional genetic modification that contributes to neuroinflammation or neurodegeneration comprises mutations or expression of transgenic molecules that lead to overexpression of a mutated amyloid precursor protein (APP) present in familial Alzheimer's disease (FAD).

68. The method of claim 67, wherein said mutated amyloid precursor protein comprises at least one of K670N, M671L, I716V, and V717I in relation to human APP(695).

69. The method of claim 68, wherein said mouse model transgenically expresses mutant human APP(695) comprising all of the following mutations: K670N, M671L, I716V, and V717I.

70. The method of claim 69, wherein expression of mutant human APP(695) is regulated by a tissue-specific promoter that is expressed in the central nervous system.

71. The method of claim 70, wherein expression of mutant human APP(695) is under the regulation of the murine Thy1 promoter.

72. The method of any one of claims 67-71, wherein said mouse model transgenically expresses mutant human presinilin 1 comprising a M146L mutation and a L286V mutation.

73. The method of claim 72, wherein expression of mutant human presinilin 1 is regulated by a tissue-specific promoter that is expressed in the central nervous system.

74. The method of claim 73, wherein expression of mutant human presinilin 1 is under the regulation of the murine Thy1 promoter.

75. The method of any one of claims 67-74, wherein said mouse model comprises a 5×FAD transgenic mouse transgenically expressing a mutant human APP(695) with the following mutations: K670N, M671L, I716V, and V717I and transgenically expressing a mutant human presinilin 1 comprising a M146L mutation and a L286V mutation.

76. The method of any one of claims 56-75, wherein said microglial LANDO knockdown or knockout increases penetrance or neuroinflammation or neurodegeneration, reduces age of onset of neuroinflammation or neurodegeneration, or both increases penetrance and reduces age of onset of neuroinflammation or neurodegeneration, when compared to a mouse lacking microglial LANDO knockdown or knockout.

77. A mouse model of neuroinflammation or neurodegeneration produced by the method of any one of claims 56-76.

78. A method for identifying a compound that modulates neuroinflammation or neurodegeneration, said method comprising:
a) administering a candidate compound to said mouse model of any one of claims 37-55 or 77;
b) measuring the effect of said candidate compound on neuroinflammation or neurodegeneration as compared to said mouse model prior to administration of said candidate compound or said mouse model not having been administered said candidate compound; and
c) selecting compounds that modulate neuroinflammation or neurodegeneration.

79. The method of claim 78, wherein measuring the effect of said candidate compound on neuroinflammation or neurodegeneration comprises measuring any one of: expression of pro-inflammatory genes, β-amyloid deposition or plaque formation, tau hyperphosphorylation, microglial activation, microglial ramified to ameboid transition, microgliosis, neuronal cell death, electrophysiological impairment, behavior deficits, and memory deficits.

80. The method of claim 79, wherein expression of any one of the following pro-inflammatory genes are measured: IL-1β, IL-6, CCL5, and TNFα.

81. The method of claim 79, wherein said microglial activation is measured by measuring expression of Iba1.

82. The method of claim 79, wherein behavior deficits are measured using a sucrose preference test.

83. The method of claim 79, wherein memory deficits are measured using a novel object recognition test, a Y-maze test, or both.

84. Use of a pharmaceutical composition that activates or enhances the LANDO pathway in the manufacture of a medicament for decreasing neuroinflammation or neurodegeneration or treating Alzheimer's disease according to the methods of claims 1-6 or 7-10, respectively.
